(19) United States
(12) Patent Application Publication
(10) Pub. No.: US 2023/0340419 A1
(43) Pub. Date: Oct. 26, 2023

(54) METHODS FOR GENERATION OF MOUSE AND HUMAN URETERIC BUD ORGANOIDS AND COLLECTING DUCT ORGANOIDS

(71) Applicant: University of Southern California, Los Angeles, CA (US)

(72) Inventors: Zhongwei Li, Los Angeles, CA (US); Biao Huang, Los Angeles, CA (US); Andrew McMahon, Los Angeles, CA (US); Zipeng Zeng, Los Angeles, CA (US)

(73) Assignee: University of Southern California, Los Angeles, CA (US)

(21) Appl. No.: 17/920,313

(22) PCT Filed: Apr. 27, 2021
(86) PCT No.: PCT/US2021/029390

§ 371 Date: Oct. 20, 2022

Related U.S. Application Data
(60) Provisional application No. 63/016,225, filed on Apr. 27, 2020.
(60) Provisional application No. 63/163,676, filed on Mar. 19, 2021.

(51) Int. Cl. C12N 5/071

(52) U.S. Cl. CPC C12N 5/0684; C12N 2506/25; C12N 2501/13; C12N 2501/10; C12N 2500/33; C12N 2500/25; C12N 2501/999; C12N 2503/02

(57) ABSTRACT

Current kidney organoids model development and diseases of the nephron but not the con-tiguous epithelial network of the kidney's collecting duct (CD) system. Here, we report the generation of an expandable, 3D branching ureteric bud (UB) organoid culture model that can be derived from primary UB progenitors from mouse and human fetal kidneys, or gen-erated de novo from human pluripotent stem cells. In chemically-defined culture conditions, UB organoids generate CD organoids, with differentiated principal and intercalated cells adopting spatial assemblies reflective of adult kidney's collecting system. Aggregating 3D-cultured nephron progenitor cells with UB organoids results in a reiterative process of branching morphogenesis and nephron induction, similar to kidney development. Applying a gene editing strategy to remove RET activity, we demonstrate genetically modified UB organoids can model congenital anomalies of kidney and urinary tract (CAKUT). These platforms facilitate an understanding of development, regeneration and diseases of the mammalian collecting system.

### CROSS-REFERENCE TO RELATED APPLICATIONS

This application includes a claim of priority under 35 U.S.C. 119(e) to U.S. provisional patent application No. 63/016,225, filed Apr. 27, 2020, and to U.S. provisional patent application No. 63/163,676, filed Mar. 19, 2021, the entirety of both of which is hereby incorporated by reference.

### STATEMENT REGARDING FEDERALLY SPONSORED RESEARCH OR DEVELOPMENT

This invention was made with government support under Grant Nos. DK054364 and DK107216 awarded by the National Institutes of Health. The government has certain rights in the invention

### REFERENCE TO SEQUENCE LISTING

The Sequence Listing submitted Apr. 27, 2021 as a text file named “SequenceListing-065715-000112WO00_ST25” created on Apr. 27, 2021 and having a size of 16,163 bytes, is hereby incorporated by reference.

### TECHNICAL FIELD

The present invention relates to kidney ureteric bud (UB) organoids and the derivative collecting duct (CD) organoids, their preparation methods and uses in modeling diseases, drug screening, and beyond.

### BACKGROUND

Three-dimensional (3D) multicellular mini-organ structures, or organoids, have broad applica-tions for modeling organ development and disease, and for regenerating organs through cell or tissue replacement therapies. However, despite previous efforts towards the expansion or de novo generation of the immature UB relying on primary mouse/rat tissue, mouse embryonic stem cells or human pluripotent stem cells, we still lack a robust kidney organoid model that can generate and expand the UB progenitor cells, and recapitulate the maturation and spatial patterning of the adult CD.

Despite the exciting progress and the wide applications, some limitations of the current kidney organoid models exist. Different segments of the nephron, the functional unit of the kidney, are identified in the kidney organoid, but the appropriate patterning of the segments, which is key to its functionality, is still difficult to achieve. A better vasculature network is needed to better recapitulate the functionality of the kidney as a blood filtration device. And similar to other stem cell-derived organoids, the immaturity is also a concern for the kidney organoid. More importantly, indispensable kidney component—the ureteric epithelium network—is almost completely missing in the existing kidney organoid models.

The mammalian kidney contains thousands of nephrons, connected to a highly branched collect-ing duct (CD) system. Nephrons filter and process the blood to form the primitive urine, which is col-lected and further refined in the CD system to adjust water, electrolytes and pH and to maintain the ho-meostasis of tissue fluid. The complex and elaborate kidney is largely formed from the reciprocal interactions of two embryonic cell populations: the epithelial ureteric bud (UB); and the surrounding metanephric mesenchyme (MM). Signals from the MM induce the repeated branching of UB, which gives rise to the entire CD network. Meanwhile, signals from the UB induce the MM to form nephrons.

The ureteric epithelium is derived from the ureteric bud (UB), which branchs and matures to form the whole collecting duct (CD) network; while within the MM, the mesenchymal nephron progenitor cells (NPCs), capping the UB tip, form nephrons, the functional units of the kidney. CD, the mature ureteric epithelium, consists of principal cell and intercalated cell, forms the urinary track to drain the urine, regulates water and electrolyte balance, and maintains the optimal pH.

Given this central role of the UB in kidney organogenesis, defects in the UB/CD development frequently lead to malformation of the kidney, low endowment of nephrons at birth, and congenital anomalies of kidney and urinary tract (CAKUT). However, despite its importance, there is currently a lack of organoid model to recapitulate the branching morphogenesis of the kidney and its maturation into the kidney collecting network. Thus, a better understanding of kidney branching morphogenesis is needed for in vitro efforts towards rebuilding the kidney. It is also required for developing novel preventive, diagnostic and therapeutic approaches for various kidney diseases.

Therefore it is an objective of the present invention to provide methods and reagents for preparing organoid models to recapitulate the branching morphogenesis of the kidney and its maturation into the kidney collecting network.

It is another objective of the present invention to provide systems and methods for studying kidney development and disease modeling, promoting kidney regeneration, and screening candidate agents to inhibit or treat a renal condition.

### SUMMARY OF THE INVENTION

Methods for generating ureteric bud (UB) organoids, or inducing the formation of UB organoids or UB-like structure, are provided, which includes culturing UB progenitor cells (UPCs) in a defined UB culture medium, so as to induce branching morphogenesis and thereby forming UB organoids that are homogeneously expressing, or at least 99%, 98%, 97%, 96%, or 95% of cells therein are positive for, one or more markers for UPCs, one or more UPC regulators, and/or one or more UB lineage markers.

In some embodiments, the UPCs are human UPCs, and the UB culture medium comprises a basal medium and supplements, wherein the supplements includes an effective amount of LDN-193189, an effective amount of TTNPB, an effective amount of CHIR99021, an effective amount of JAK inhibitor I, an effective amount of GDNF, an effective amount of A83-01, an effective amount of R-spondin 1, an effective amount of fibroblast growth factor (FGF) 7, an effective amount of SB202190, and an effective amount of epidermal growth factor (EGF).

In some embodiments, the UPCs are mouse UPCs, and the UB culture medium comprises a basal medium and a combination of supplements, wherein the combination of supplements includes an effective amount of FGF9, an effective amount of TTNPB, an effective amount of CHIR99021, an effective amount of GDNF, an effective amount of LDN-193189, an effective amount of A83-01, an effective amount of JAK Inhibitor I, an effective amount of SB202190, and an effective amount of R-Spondin 1.

In further embodiments, the generated UB organoids can be passaged for an extended period of time. For example, the tip cells in the UB organoids can be resected and separately cultured in the UB culture medium to induce UB organoid formation.

Methods for generating UPCs from pluripotent stem cells (PSCs) are also provided, which include culturing the PSCs in the (sequential) presence of (1) an effective amount of mTeSR™1 medium (TeSR) and CloneR (CR), (2) ME medium comprising supplements of LDN-193189 and CHIR99021, (3) UB-I medium comprising supplements of FGF2, TTNPB, LDN-193189, and A83-01, (4) UB-II medium comprising supplements of FGF2, TTNPB, and LDN-193189. After about 7 days, the cultivated PSCs can be sorted to identify KIT+ cells, so as to isolate and obtain UPCs.

Methods for generating a collecting duct (CD) organoid, or inducing formation of CD organoids, are provided, which include culturing a UB organoid in a CD differentiation medium; or inducing the formation of UPCs from PSCs, followed by generating a UB organoid with the UPCs, and then culturing the UB organoid in a CD differentiation medium; so as to obtain a CD organoid. In further embodiments, the UB organoid is cultured in the CD differentiation medium for 7 days or longer to form an elongated CD morphology, and characterized by elevated expressions of a principal cell (PC)-specific marker and/or an intercalated cell (IC)-specific marker.

In some embodiments, the UB organoids are human UB organoids, and the CD differentiation medium comprises supplements of aldosterone, vasopressin, and KNOCKOUT serum replacement (KSR).

In some embodiments, the UB organoids are mouse UB organoids, and the CD differentiation medium comprises supplements of FGF9, Y27632, DAPT, PD0325901, aldosterone, and vasopressin.

Methods for generating an engineered kidney are also provided, which include combining the tip cells of an UB organoid with nephron progenitor cells in one culture, and cultivating the combination in a kidney reconstruction medium, so as to obtain a tubular network with connected nephron-like cell types and a collecting duct.

In some embodiments, the kidney reconstruction medium comprises all, or one or more, of: TTNPB, APEL2, and Y27632.

Methods for screening a candidate compound for therapeutic efficacy in treating a kidney disease or disorder, or for nephrotoxicity, are also provided, which include contacting the candidate compound with an UB organoid, a CD organoid, or an engineered kidney, and measuring the activity of a marker in the UB organoid, the CD organoid, or the engineered kidney, or measuring or assessing for a nephrotoxic side effect.

An UB organoid, a CD organoid, and an engineered kidney, generated by the methods disclosed herein, are also provided.

Also provided is an assay or a kit for the screening, which includes an UB organoid, a CD organoid, and an engineered kidney in culture.

Compositions for UB culture medium, for CD culture medium, or for kidney reconstruction medium, are also provided.

### BRIEF DESCRIPTION OF THE DRAWINGS

The foregoing and other advantages of the present disclosure will become apparent upon reading the following detailed description and upon reference to the drawings, which are appended hereto.

FIG. 1A-1F depict expanding mouse UB progenitor cells into 3D branching UB organoids. 1A, Schematic of mouse UB isolation and screening for UB organoid culture condition. 1B, Representative bright field (BF, left panel) and Wnt11-RFP (right panel) images of UB organoid. Scale bars, 200 μm. 1C, Cumulative growth curve of UB organoid culture starting from 2,000 cells. Each time point represents 3 biological replicates. 1D, Quantification of percentages of UB cells stained positive for different UB markers. Each column represents counts from at least 3 different fields of view. 1E, Unsupervised clustering analysis of RNA-seq data. 1F, Principal component analysis (PCA) of RNA-seq data. Different colors and oval cycles represent different primary kidney cell populations (NPC, IPC, UB tip, and UB trunk) or UB organoids cultured for 5 days (D5), 10 days (D10), and 20 days (D20).

FIG. 1G depicts the efficiency of mouse UB organoid formation from 200 single UB cells. Each group represents 3 biological replicates. All data are presented as mean±s.d.

FIG. 1H shows representative images of UB organoid derivation from a single UB cell-derived budding structure isolated from the boxed region in (h) at day 0 (DO, the day of isolation and re-embedding into Matrigel), day 2 (D2), day 4 (D4) and day 6 (D6). Scale bar, 400 μm. All data are presented as mean±s.d.

FIG. 2A-2G depict the generation of mature and highly organized CD organoids from UB organoids. 2A, Schematic of the screening strategy for identifying differentiation culture condition to generate CD organoids from UB organoids. 2B, Bright field images showing the morphologic differences between UB organoid and CD organoid. 2C, qRT-PCR analyses of UB (red) and CD (green) organoids for UB progenitor markers Wnt11 and Ret, PC markers Aqp2 and Aqp3, IC markers Foxi1, Atp6v1b1, Slc4a1, and Slc26a4, and Tfcp2l1 that is expressed in both PC and IC. Adult mouse kidney (blue) was used as control. The significance was determined by two-tailed unpaired Student's t-tests; ***, P<0.001. 2D, Whole-mount immunostaining analyses of CD organoids for PC marker AQP2, and IC markers FOXI1 and ATP6V1B1, showing the distribution of these two cell types within the organoid; scale bar=25 μm; 2E, Immunostaining of cryo-section samples of differentiated CD organoids for ureteric lineage marker (GATA3) and various PC (AQP2 and AQP3). Note the sporadic distribution of the IC in the organoid. Scale bars=50 μm. 2F, Comparison of PC and IC ratios in postnatal day 0 (PO) mouse CD, adult mouse CD, and CD organoids. Whole-mount immunostaining images (CD organoids), or section staining images (PO and adult kidneys) stained for AQP2 (PC) and FOXI1 (IC) were quantified for ratios of PC and IC in the CD organoid or the kidney's collecting duct. Each column represents counts from at least 3 different fields of view. 2G, Principal component analysis (PCA) of RNA-seq data. Different colors and oval cycles represent different primary kidney cell populations (NPC, IPC, UB tip, UB trunk, and adult CD cells), or UB organoids cultured for 5 days (D5), 10 days (D10), and 20 days (D20), or CD organoids. All data are presented as mean±s.d.

FIG. 3A-3G depict the generation of engineered kidney from expandable NPCs and UBs and gene editing of the UB organoid. 3A, Schematic of the engineered kidney reconstruction and organotypic culture procedure. 3B, Time course images (bright field and Hoxb7-Venus) showing the branching morphogenesis of the engineered kidney reconstructed at air-liquid interface at day 2, day 4, day 5, and day 7. Scale bars, 200 μm. 3C, Immunostaining of the engineered kidney (Day 7) constructed from Wnt11-RFP UB organoid and wild-type NPCs for UB/CD marker KRT8, nephron marker PODXL and WT1 (podocytes) and LTL (proximal tubule). Note that both KRT8 and PODXL were stained green. The round structures that co-stain with WT1 are podocytes of the nephron. UB-derived structures do not co-stain with WT1. Scale bar, 100 μm. 3D, Immunostaining of the engineered kidney constructed from Hoxb7-Venus UB organoid and wild-type NPC (Day 10) for GATA3 and CDH1. Scale bars, 50 μm. 3E, Schematic of gene overexpression and gene knockout procedures in the UB organoid. OE, overexpression; KO, knockout. 3F, Fluorescence image of GFP overexpression (GFP OE) in wild-type UB organoid. Scale bar=200 μm. 3G, Knockout of GFP in Rosa26-Cas9/GFP UB organoid using multiplexed sgRNAs (“GFP KO”, right panels) targeting the coding sequence of GFP. Multiplexed non-targeting sgRNAs were introduced to the organoid as control (“Ctrl KO”, left panel). Note the gene-edited single cells self-organized into typical branching organoid morphology. Scale bars=400 μm.

FIG. 4A-4G depict the generation of human UB and CD organoids from primary human UPCs and dual-reporter human pluripotent stem cells. 4A, Schematic of the purification of primary human UPCs from the nephrogenic zone (illustrated as boxed region) of the human fetal kidney (9-13 weeks of gestational age) and the derivation of human UB organoid. 4B, Time course bright field images showing the growth of human UB organoid derived from primary human UPCs in atypical passage cycle at day 1 (D1), day 5 (D5), and day 9 (D9). Scale bar, 200 μm. 4C, qRT-PCR analyses of human UB organoid (cultured for 54 days) derived from primary human UPCs for various UB markers as indicated. Human fetal kidney from 11.2-week (11.2 wk) gestational age was used as control. 4D, Schematic of the stepwise differentiation from WNT11-GFP/PAX2-mCherry dual reporter hESC line into iUB and iCD organoid. (TeSR, mTeSR1 medium; Y, Y27632; ME, mesendoderm stage medium; UB-I, UB Stage I medium; UB-II, UB Stage II medium). 4E, qRT-PCR analyses of the FACS purified mCherry⁺ cells (orange) and the iUB organoid (blue, cultured for 50 days) for various UB markers as indicated. Undifferentiated H1 hESCs (gray) and human fetal kidney (green, 11.2 week gestational age) were used as controls. 4F, Bright field (BF) and PAX2-mCherry (PAX2-mCh) images of expandable iUB organoid (left panels) and mature iCD organoid (right panels). Scale bars=200 μm. 4G, qRT-PCR analyses of the iUB organoid (orange, cultured for 49 days) and iUB-derived iCD organoid (blue), for UB markers (WNT11, ETV5), PC markers (AQP2, AQP4), and IC marker (FOXI1). In 4C, 4E, and 4G, the significance was determined by two-tailed unpaired Student's t-tests; NS, not significant; *, P<0.05; **, P<0.01; ***, P<0.001.

FIG. 4H-4J depict the derivation and expansion of iUB organoid from SOX9-GFP hiPSC. 4H, Bright field images of branching iUB organoid derived from SOX9-GFP reporter hiPSCs in a typical passage cycle at day 0 (DO). 4I, Bright field images of branching iUB organoid derived from SOX9-GFP reporter hiPSCs in a typical passage cycle at day 0 (DO) and day 9 (D9). 4J, SOX9-GFP fluorescence of branching iUB organoid derived from SOX9-GFP reporter hiPSCs in a typical passage cycle at day 0 (DO) and day 9 (D9). The indicated budding structure shown in 4H was dissected and re-embedded into Matrigel for iUB expansion shown in 4I and 4J. Scale bars=200 μm.

FIG. 5A-5G depict the generation of human iUB and iCD organoids from human pluripotent stem cells independent of reporters. 5A, Schematic of the stepwise differentiation from any hPSC line into iUB and iCD organoid without using reporter. (TeSR, mTeSR1 medium; CR, CloneR; ME (v2), mesendoderm stage medium version 2; UB-I, UB Stage I medium; UB-II, UB Stage II medium). 5B, qRT-PCR analyses of the FACS purified KIT⁺ precursor (orange), KIT⁺ precursor-derived iUB organoids cultured for 33 days (D33, gray), 49 days (D49, yellow), and 66 days (D66, light blue) for various UB markers as indicated. Undifferentiated H1 hESCs (dark blue) and human fetal kidney (green, 11.2 week gestational age) were used as controls. 5C, Cumulative growth curve of iUB organoid culture starting from 20,000 cells at day 20. Each time point represents 3 biological replicates. 5D, Quantification of percentages of iUB cells stained positive for different UB markers in FIG. 5c-e. Each column represents counts from at least 3 different fields of view. 5E, qRT-PCR analyses of the iUB organoid (blue) and iUB-derived iCD organoid (orange), for UB markers (WNT11, RET), PC markers (AQP2, AQP3, AQP4), and IC marker (FOXI1). 5F, Bright field and fluorescence images showing the morphological changes and decreasing of WNT11-GFP from iUB organoid (left panels) to mature iCD organoid (right panels). Scale bars=200 μm. 5G, qRT-PCR analyses of the iUB organoids derived from the dual reporter hESC line (W/P reporter, orange, cultured for 33 days) or wild-type H1 hESC line (H1, gray, cultured for 30 days) for various UB markers. Undifferentiated H1 hESCs (blue) and human fetal kidney (yellow, 11.2 week gestational age) were used as controls. All data are presented as mean±s.d. The significance was determined by two-tailed unpaired Student's t-tests; *, P<0.05; **, P<0.01; ***, P<0.001.

FIG. 6A-6D depict a modeling of kidney development and disease using mouse and human UB organoids. 6A, Schematic of Ret/RET gene knockout procedures in the mouse or human UB organoid. 6B, Bright field images showing the branching morphogenesis of mouse UB organoids 2 days (Day 2) and 6 days (Day 6) after lentiviral infection. Scale bars, 200 μm. 6C, qRT-PCR analyses of the control mouse UB organoids (blue and orange) and Ret KO mouse UB organoids (gray and yellow) for various UB markers 6 days after lentiviral infection. 6D, Bright field images showing the branching morphogenesis of human iUB organoids 3 days (Day 3) and 12 days (Day 12) after lentiviral infection. Scale bars=200 μm.

While the present disclosure is susceptible to various modifications and alternative forms, specific implementations have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

Unless otherwise specified, the term “about” a value is intended to include any value within the range of ±5% of that value. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

The phrase “pluripotent stem cell(s)” refers to stem cells which have pluripotency, that is the ability of cells to differentiate into all types of the cells in the living body, as well as proliferative capacity. Examples of the pluripotent stem cells include embryonic stem (ES) cells, embryonic stem cells derived from cloned embryo obtained by nuclear transfer, germline stem cells, embryonic germ cells, induced pluripotent stem (iPS) cells, pluripotent cells derived from cultured fibroblasts and bone marrow stem cells. Mouse or human pluripotent stem cells, particularly ES cells and iPS cells are preferably used.

TTNPB is a retinoic acid receptor (RAR) agonist not having the retinoid structure. TTNPB is 4-[[E]-2-[5,6,7,8-tetrahydro-5,5,8,8-tetramethyl-2-naphthalenyl]-1-propenyl]benzoic acid. In various embodiments, TTNPB is included in the compositions, or used in the methods, in place of another RAR agonist, such as naturally-occurring retinoid, chemically synthesized retinoid, a naturally occurring substance having the RAR aonist activity, or another RAR aonist not having the retinoid structure. Examples of naturally occurring retinoid having the retinoic acid receptor agonist activity include retinoic acid such as known stereoisomers, all-trans retinoic acid (all-trans RA) and 9-cis retinoic acid (9-cis RA). Chemically synthesized retinoid is known to the art (for example, U.S. Pat. Nos. 5,234,926 and 4,326,055). Examples of retinoic acid receptor agonists not having the retinoid structure include Am80, AM580, TTNPB, and AC55649. Examples of naturally occurring substances having the retinoic acid receptor agonist activity include honokiol and magnolol.

Unless otherwise specified, in each step of the methods, the cells may be cultured at a temperature of about 30-40° C., preferably about 37° C. under a CO₂-containing air atmosphere, but not limited to such conditions. The concentration of CO₂ in the air may preferably be about 2-5%. Unless otherwise specified, the medium for cell culturing can be prepared by appropriately adding factors (or “supplements”) necessary for each stage to a basal medium used for culturing animal cells. Examples of the basal media include Dulbecco's modified Eagle's Medium (DMEM) Medium, DMEM/F12 Medium, MEM Zinc Option Medium, IMEM Zinc Option Medium, IMDM Medium, Medium 199 Medium, Eagle's Minimum Essential Medium (EMEM) Medium, α-MEM Medium, Ham's F12 Medium, RPMI 1640 Medium, Fischer's Medium, and mixtures of these media. The basal medium may contain serum (for example, fetal bovine serum (FBS)) or the basal medium may be a serum-free medium. As required, the basal medium may contain, for example, one or more alternatives to sera such as KnockOut Serum Replacement (KSR) (Thermo Fisher Scientific), which is an alternative to serum used for culturing ES cells, albumin, transferrin, N2 Supplement (Thermo Fisher Scientific), B-27 Supplement (Thermo Fisher Scientific), a fatty acid, insulin, a collagen precursor, a trace element, 2-mercaptoethanol, and 3′-Thioglycerol, and the basal medium may also contain one or more substances such as a lipid, an amino acid, L-glutamine, GlutaMAX (Thermo Fisher Scientific), a nonessential amino acid (NEAA), a vitamin, a growth factor, an antibiotic, an antioxidant, pyruvic acid, a buffer agent, an inorganic salt, and equivalents thereof as well as one or more other substances.

The phrase “branching morphogenesis,” or a result “branching morphology,” encompasses the numerous cellular process involved in the formation, or as a result thereof, respectively, of branched networks, including proliferation, survival/apoptosis, migration, invasion, adhesion, aggregation and matrix remodeling. The terms “cells” and “population of cells” are used interchangeably and refer to a plurality of cells, i.e., more than one cell. The population may be a pure population comprising one cell type. Alternatively, the population may comprise more than one cell type.

The term “contacting” can refer to bringing a disclosed composition, compound, or complex together with an intended target (such as, e.g., a cell or population of cells, a receptor, an antigen, or other biological entity) in such a manner that the disclosed composition, compound, or complex can affect the activity of the intended target (such as, e.g., a cell or population of cells, a receptor, an antigen, or other biological entity).

The term “candidate compound/drug” or “a compound/drug of interest” refers to an agent to be screened. Candidate compounds may include, for example, small molecules such as small organic compounds (e.g., organic molecules having a molecular weight between about 50 and about 2,500 Da), peptides or mimetics thereof, ligands including peptide and non-peptide ligands, polypeptides, nucleic acid molecules such as aptamers, peptide nucleic acid molecules, and components, combinations, and derivatives thereof.

The “lineage” of a cell defines the heredity of the cell, i.e., which cells it came from and what cells it can give rise to. The lineage of a cell places the cell within a hereditary scheme of development and differentiation.

The term “organoid” generally refers to an agglomeration of cells that recapitulates aspects of cellular self-organization, architecture and signaling interactions present in a native organ. The term “organoid” includes spheroids or cell clusters formed from suspension cell cultures. In some embodiments, an organoid comprises a number in the order of 10⁴, 10⁵, or 10³ cells.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

A cell that is referred to as being “positive” for a given marker may express a level of that marker depending on the degree to which the marker is present on the cell surface. In some embodiments, the term relates to intensity of fluorescence or other marker used in the sorting process of the cells. In some embodiments, a cell may express a low level or a bright level of a marker, and the distinction of low and bright will be understood in the context of the marker used on a particular cell population being sorted. A cell that is referred to as being “negative” for a given marker can mean that that given marker is absent from that cell, or can also mean that the marker is expressed at a relatively low or very low level by that cell or population, and that it generates a very low signal when detectably labelled or is undetectable above background levels.

In some embodiments, expression levels can be measured using techniques such as polymerase chain reaction comprising appropriate primers for markers of interest. For example, total RNA can be extracted from organoids before being reverse transcribed and subject to PCR and analysis.

In various embodiments, a positive marker refers to an expression of the corresponding gene and/or a level of the corresponding protein above a reference, control or background level. Generally, standard gene names and symbols can be found in community databases specific to particular organisms (e.g., human: www.genenames.org; rat: rgd.mcw.edu; mouse: www.informatics.jax.org; zebrafish: zfin.org; flies: flybase.org; worms: www.wormbase.org). In general, symbols for genes are italicized (e.g., IGF1), whereas symbols for proteins are not italicized (e.g., IGF1); and gene names that are written out in full are not italicized (e.g., insulin-like growth factor 1). For humans, non-human primates, chickens, and domestic species, gene symbols contain three to six italicized characters that are all in upper-case (e.g., RET). For mice and rats, gene symbols are italicized, with only the first letter in upper-case (e.g., Ret). Gene symbol RET refers to ret proto-oncogene; gene symbol WNT11 refers to Wnt family member 11; gene symbol ETV5 refers to ETS variant transcription factor 5; gene symbol SOX9 refers to SRY-box transcription factor 9; gene symbol GATA3 refers to GATA binding protein 3; gene symbol PAX2 refers to paired box 2; gene symbol KRT8 refers to keratin 8; gene symbol CDH1 refers to cadherin 1; gene symbol AQP2 refers to aquaporin 2; gene symbol AQP3 refers to aquaporin 3; gene symbol FOXI1 refers to forkhead box I1; gene symbol ATP6V1B1 refers to ATPase H+ transporting V1 subunit B1; gene symbol SLC4A1 refers to solute carrier family 4 member 1; gene symbol SLC26A4 refers to solute carrier family 26 member 4, with an alias gene name: pendrin;

Here, the inventors report the generation of three-dimensional (3D) branching UB organoid from mouse and human primary UB progenitor cells, as well as from human pluripotent stem cells. The expandable UB organoids maintained the branching morphology and showed molecular homogeneity of UB progenitor features. The combination of 3D cultured nephron progenitor cells with UB organoid restored kidney organogenesis and reconstructed a branched synthetic kidney in vitro. Screening based on the UB organoid further identified method to differentiate UB organoid into CD organoid with properly patterned mature principal and intercalated cells. Combined with efficient gene editing in the UB organoids, the invention provides a powerful technological platform for, among other things, studying kidney regeneration and disease modeling.

The present invention is based, at least in part, on the inventors' development of a novel 3D organoid model that mimics the full spectrum of kidney branching morphogenesis in vitro from the immature UB progenitor stage, to the mature CD stage, starting from either primary UB progenitor cells, or human pluripotent stem cells. These technological platforms provide novel tools for studying kidney development, regeneration and disease modeling.

In an embodiment, the present invention relates to a platform for generating expandable, branching and gene-editable ureteric bud organoid from primary mouse and human ureteric bud progenitor cells and human pluripotent stem cells, and its maturation into collecting duct organoid.

Various embodiments provide methods for generating a renal UB organoid from UB progenitor cells (UPCs), which include cultivating UPCs in a UB culture medium to induce a branching morphology of the UPCs, thereby forming an UB organoid, wherein the UB culture medium comprises a basal medium and supplements.

Further embodiments provide methods for inducing, generating, maintaining, and rebuilding a UB-like structure (e.g., organoid) from UB cells in vitro, which include cultivating the UB cells in a UB culture medium to induce a branching morphology of the UB cells, thereby forming the UB-like structure.

In various embodiments, the supplements of the UB culture medium comprise one or more, or all of: LDN-193189, TTNPB, CHIR99021, Janus-associated kinase inhibitor I (JAK inhibitor I), glial cell-derived neurotrophic factor (GDNF), A83-01, R-spondin 1, a fibroblast growth factor (FGF), and SB202190. In some embodiments, the supplements of the UB culture medium comprise all of: LDN-193189, TTNPB, CHIR99021, Janus-associated kinase inhibitor I (JAK inhibitor I), glial cell-derived neurotrophic factor (GDNF), A83-01, R-spondin 1, a fibroblast growth factor (FGF), and SB202190.

In further embodiments, the supplements of the UB culture medium further comprise one or more, or all of L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution. That is, the supplements of the UB culture medium comprise, cosist essentially of, or consist of LDN-193189, TTNPB, CHIR99021, JAK inhibitor I, GDNF, A83-01, R-spondin 1, an FGF, SB202190, L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution. In further embodiments, the basal medium of the UB culture medium is DMEM medium, or DMEM/F12 (1:1). In some embodiments, the supplements of the UB culture medium further comprise all of L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution.

In further embodiments of the UB culture medium, the TTNPB is included to replace retinoic acid (RA); and so the UB culture medium does not comprises retinoic acid and/or is not supplemented with retinoic acid.

In a further embodiment of the UB culture medium, the CHIR99021 is within 1-6 μM. In a further embodiment of the UB culture medium, the CHIR99021 ranges from 2 μM to 4 μM. In a further embodiment of the UB culture medium, the CHIR99021 is preferably 3 μM or about 3 μM.

In some embodiments, the methods for generating a renal UB organoid from human UPCs or human UB cells comprise culturing the human cells in a UB culture medium to induce a branching morphology of the UPCs, thereby forming an UB organoid, wherein the UB culture medium comprises supplements of one or more, or all of LDN-193189, TTNPB, CHIR99021, JAK inhibitor I, GDNF, A83-01, R-spondin 1, fibroblast growth factor (FGF) 7 (FGF7), SB202190, and epidermal growth factor (EGF). In some embodiments of generating a renal UB organoid from human UPCs or human UB cells, wherein the UB culture medium comprises supplements of all of LDN-193189, TTNPB, CHIR99021, JAK inhibitor I, GDNF, A83-01, R-spondin 1, fibroblast growth factor (FGF) 7 (FGF7), SB202190, and epidermal growth factor (EGF).

In a further embodiment, the UB culture medium for culturing the human UPCs or human UB cells does not comprises, or is not supplemented with Y27632. In some embodiments, the FGF7 is included in the supplements of the UB culture medium for cultivating the human cells is to replace FGF1 or another FGF; and so the UB culture medium does not comprises FGF1 and/or is not supplemented with FGF1.

In some embodiments, the methods for generating a renal UB organoid from mouse UPCs or mouse UB cells comprise culturing the human cells in a UB culture medium to induce a branching morphology of the UPCs, thereby forming an UB organoid, wherein the UB culture medium comprises supplements of one or more, or all of FGF9, TTNPB, CHIR99021, GDNF, LDN-193189, A83-01, JAK Inhibitor I, SB202190, and R-Spondin 1. In a further embodiment, the UB culture medium for culturing the mouse UPCs or mouse UB cells does not comprises, or is not supplemented with EGF. In some embodiments, the FGF9 is included in the supplements of the UB culture medium for cultivating the mouse cells is to replace FGF1; and so the UB culture medium does not comprises FGF1 and/or is not supplemented with FGF1. In some embodiments of generating a renal UB organoid from mouse UPCs or mouse UB cells, wherein the UB culture medium comprises supplements of all of FGF9, TTNPB, CHIR99021, GDNF, LDN-193189, A83-01, JAK Inhibitor I, SB202190, and R-Spondin 1.

In some embodiments, the LDN-193189 in the UB culture medium for cultivating UPCs or UB cells, (human or mouse sourced,) is at a final concentration of 200 nM or about 200 nM. In some embodiments, the LDN-193189 in the UB culture medium is between 50-500 nM. In some embodiments, the LDN-193189 in the UB culture medium is between 100-400 nM. In some embodiments, the LDN-193189 in the UB culture medium is between 150-300 nM. In some embodiments, the LDN-193189 in the UB culture medium is between 170-250 nM. In some embodiments, the LDN-193189 in the UB culture medium is between 180-230 nM. In some embodiments, the LDN-193189 in the UB culture medium is between 190-220 nM.

In some embodiments, the TTNPB in the UB culture medium for cultivating UPCs or UB cells, (human or mouse sourced,) is at a final concentration of 0.1 μM or about 0.1 μM. In some embodiments, the TTNPB in the UB culture medium is between 0.02-0.5 μM. In some embodiments, the TTNPB in the UB culture medium is between 0.04-0.4 μM. In some embodiments, the TTNPB in the UB culture medium is between 0.06-0.3 μM. In some embodiments, the TTNPB in the UB culture medium is between 0.08-0.2 μM. In some embodiments, the TTNPB in the UB culture medium is between 0.09-0.15 μM.

In some embodiments, the CHIR99021 in the UB culture medium for cultivating UPCs or UB cells, (human or mouse sourced,) is at a final concentration of 3 μM or about 3 μM. In some embodiments, the CHIR99021 in the UB culture medium is between 0.5-9 μM. In some embodiments, the CHIR99021 in the UB culture medium is between 1-6 μM. In some embodiments, the CHIR99021 in the UB culture medium is between 2-5 μM. In some embodiments, the CHIR99021 in the UB culture medium is between 2.5-4 μM. In some embodiments, the CHIR99021 in the UB culture medium for cultivating induced pluripotent stem cell-derived human UPCs (or human UB cells) is at a final concentration of 1 μM or about 1 μM; or between 0.5-3 μM, between 0.6-2 μM, between 0.7-1.5 μM, or between 0.8-1.2 μMM.

In some embodiments, the JAK inhibitor I in the UB culture medium for cultivating UPCs or UB cells, (human or mouse sourced,) is at a final concentration of 100 nM or about 100 nM. In some embodiments, the JAK inhibitor I in the UB culture medium is between about 50-300 nM. In some embodiments, the JAK inhibitor I in the UB culture medium is between 60-250 nM. In some embodiments, the JAK inhibitor I in the UB culture medium is between 70-200 nM. In some embodiments, the JAK inhibitor I in the UB culture medium is between 80-150 nM. In some embodiments, the JAK inhibitor I in the UB culture medium is between 90-120 nM.

In some embodiments, the GDNF in the UB culture medium for cultivating human UPCs or human UB cells is human GDNF, and the GDNF in the UB culture medium for cultivating mouse UPCs or mouse UB cells is mouse GDNF; and the GDNF is at a final concentration of 50 ng/mL or about 50 ng/mL. In some embodiments, the GDNF in the UB culture medium is between 10-100 ng/mL. In some embodiments, the GDNF in the UB culture medium is between 20-90 ng/mL. In some embodiments, the GDNF in the UB culture medium is between 30-80 ng/mL. In some embodiments, the GDNF in the UB culture medium is between 40-70 ng/mL. In some embodiments, the GDNF in the UB culture medium is between 45-60 ng/mL.

In some embodiments, the A83-01 in the UB culture medium for cultivating UPCs or UB cells, (human or mouse sourced,) is at a final concentration of 0.2 μM or about 0.2 μM. In some embodiments, the A83-01 in the UB culture medium is between 0.05-0.5 μM. In some embodiments, the A83-01 in the UB culture medium is between 0.1-0.4 μM. In some embodiments, the A83-01 in the UB culture medium is between 0.15-0.3 μM. In some embodiments, the A83-01 in the UB culture medium is between 0.17-0.25 μM.

In some embodiments, the R-spondin 1 in the UB culture medium for cultivating UPCs or UB cells, (human or mouse sourced,) is at a final concentration of 100 ng/mL or about 100 ng/mL. In some embodiments, the R-spondin 1 in the UB culture medium is between about 50-300 ng/mL. In some embodiments, the R-spondin 1 in the UB culture medium is between 60-250 ng/mL. In some embodiments, the R-spondin 1 in the UB culture medium is between 70-200 ng/mL. In some embodiments, the R-spondin 1 in the UB culture medium is between 80-150 ng/mL. In some embodiments, the R-spondin 1 in the UB culture medium is between 90-120 ng/mL.

In some embodiments, the SB202190 in the UB culture medium for cultivating UPCs or UB cells, (human or mouse sourced,) is at a final concentration of 5 μM or about 5 μM. In some embodiments, the SB202190 in the UB culture medium is between 1-2, 2-3, 3-4, 4-5, 5-6, 6-7, 7-8, 8-9, or 9-10 μM, or any combination thereof.

In some embodiments, the FGF in the UB culture medium for cultivating human UPCs or human UB cells is FGF7 at a final concentration of 50 ng/mL or about 50 ng/mL. In some embodiments, the FGF7 in the UB culture medium IS between 10-20, 20-30, 30-40, 40-50, 50-60, 60-70, 70-80, 80-90, or 90-100 ng/mL, or any combination thereof. In some embodiments, the FGF in the UB culture medium for cultivating mouse UPCs or mouse UB cells is FGF9 at a final concentration of 50 ng/mL or about 50 ng/Ml. In some embodiments, the FGF9 in the UB culture medium is between 10-20, 20-30, 30-40, 40-50, 50-60, 60-70, 70-80, 80-90, or 90-100 ng/mL, or any combination thereof.

In some embodiments, the EGF in the UB culture medium for cultivating human UPCs or human UB cells is at a final concentration of 50 ng/mL or about 50 ng/mL. In some embodiments, the EGF in the UB culture medium is between 10-20, 20-30, 30-40, 40-50, 50-60, 60-70, 70-80, 80-90, or 90-100 ng/mL, or any combination thereof. In further embodiments, EGF is not included in, or supplemented to, the UB culture medium for cultivating mouse UPCs or mouse UB cells.

In some embodiments, the Y27632, if present, in the UB culture medium for cultivating human UPCs or human UB cells is at a final concentration of 10 μM or about 10 μM. In some embodiments, the Y27632, if present, in the UB culture medium is between 1-2, 2-3, 3-4, 4-5, 5-6, 6-7, 7-8, 8-9, 9-10, 10-12, 12-14, 14-16, 16-18, or 18-20 μM, or any combination thereof. In further embodiments, Y27632 is not included in, or supplemented to, the UB culture medium for cultivating mouse UPCs or mouse UB cells.

In various embodiments, the generated renal UB organoid or UB-like structure comprises at least 99%, 98%, 97%, 96%, or 95% of cells, or 100% of cells, in the branching morphology that are positive in one or more markers for UPCs, one or more UPC regulators, and/or one or more UB lineage markers. In some embodiments, the markers for UPCs comprise RET and WNT11, the UPC regulators comprise RET, ETV5, and SOX9, and the UB lineage markers comprise GATA3, PAX2, KRT8, and CDH1.

In further embodiments, the generated renal UB organoid or UB-like structure can be passaged for a plurality of times, e.g., at least 2, 3, 4, 5, 6, 7, 8, 9, 10, 15, or 20 times, each passage maintaining the positive expression of the one or more markers for UPCs, one or more UPC regulators, and/or one or more UB lineage markers. In some embodiments, the passaging process can be repeated for up to 3 weeks when the population of UPCs are obtained from mouse fetal kidney, or wherein the method is repeated for at least 100 days when the population of UPCs are obtained from human fetal kidney, or wherein the method is repeated for at least 70 days when the population of UPCs are obtained from human PSCs.

In some embodiments, passaging the renal UB organoid or UB-like structure includes resecting a tip portion of cells (or UB tip cells) from the branching morphology of the UB organoid (or UB-like structure), and culturing the tip portion of cells in a fresh volume of the UB culture medium to induce branching morphology, thereby generating a subsequent passage of the UB organoid. UB tip cells are considered to be the progenitor cells. Tip and stalk regions can be manually separated, with an immunostaining for ETV5 to confirm the presence of tip cells that express ETV5.

Various embodiments provide methods for generating UPCs from pluripotent stem cells (PSCs), which include cultivating the PSCs in the presence of:
mTeSRTM1 medium (TeSR) and CloneR (CR) for a first period of time,
ME medium for a second period of time,
UB-I medium for a third period of time,
UB-II medium for a fourth period of time; and
   sorting KIT+ cells from the cultivated PSCs, thereby obtaining the UPCs.

In various embodiments, the methods for generating UPCs from pluripotent stem cells (PSCs) is a process of about 7 days, including cultivating the PSCs in four periods of time above in a sequential order. In further embodiments, the TeSR and the CR for the first period of time is about 1 day, or 0.5-2 days; the ME medium for the second period of time is about 2 days, or 1-3 days; the UB-I medium for the third period of time is about 2 days, or 1-3 days; the UB-II medium for the fourth period of time is about 2 days, or 1-3 days.

In some embodiments, the ME medium comprises or is supplemented with one or both of LDN-193189 and CHIR99021. In some embodiments, the ME medium comprises or is supplemented with both LDN-193189 and CHIR99021. In some embodiments, the ME medium comprises or is supplemented with LDN-193189 at a final concentration between 1-30 nM, or ranging from 5 nM to 15 nM, or is 10 nM or about 10 nM; and CHIR99021 at a final concentration between 1-10 μM, between 2-9 μM, between 3-8 μM, between 3-7 μM, between 3.5-6 μM, between 4-5 μM, or is 4.5 μM or about 4.5 μM.

In other embodiments, the ME medium comprises or is supplemented with one or both of Activin A and CHIR99021. In other embodiments, the ME medium comprises or is supplemented with both of Activin A and CHIR99021. In some embodiments, the ME medium comprises or is supplemented with Activin A at a final concentration between 10-100 ng/mL, between 20-80 ng/mL, between 30-70 ng/mL, between 40-60 ng/mL, between 45-55 ng/mL, or is 50 ng/mL or about 50 ng/mL; and CHIR99021 at a final concentration between 0.5-6 μM, between 1-5 μM, between 2-4 μM, between 2.5-3.5 μM, or is 3 μM or about 3 μM.

In some embodiments, the UB-I medium comprises or is supplemented with one or more, or all of FGF2, TTNPB, LDN-193189, and A83-01. In some embodiments, the UB-I medium comprises or is supplemented with all of FGF2, TTNPB, LDN-193189, and A83-01. In some embodiments, the FGF2 for the UB-I medium is at a final concentration of 200 ng/mL or about 200 ng/mL. In some embodiments, the FGF2 for the UB-I medium is between 50-500 ng/mL. In some embodiments, the FGF2 for the UB-I medium is between 100-400 ng/mL. In some embodiments, the FGF2 for the UB-I medium is between 150-300 ng/mL. In some embodiments, the FGF2 for the UB-I medium is between 180-250 ng/mL. In some embodiments, the TTNPB for the UB-I medium is at a final concentration of 0.1 μM or about 0.1 μM. In some embodiments, the TTNPB for the UB-I medium is between 0.01-0.5 μM. In some embodiments, the TTNPB for the UB-I medium is between 0.05-0.3 μM. In some embodiments, the TTNPB for the UB-I medium is between 0.07-0.2 μM. In some embodiments, the TTNPB for the UB-I medium is between 0.08-0.15 μM. In some embodiments, the LDN-193189 for the UB-I medium is at a final concentration of 30 nM or about 30 nM. In some embodiments, the LDN-193189 for the UB-I medium is between 10-100 nM. In some embodiments, the LDN-193189 for the UB-I medium is between 15-80 nM. In some embodiments, the LDN-193189 for the UB-I medium is between 20-60 nM. In some embodiments, the LDN-193189 for the UB-I medium is between 25-40 nM. In some embodiments, the A83-01 for the UB-I medium is at a final concentration of 0.2 μM or about 0.2 μM. In some embodiments, the A83-01 for the UB-I medium is between 0.05-1 μM. In some embodiments, the A83-01 for the UB-I medium is between 0.1-0.5 μM. In some embodiments, the A83-01 for the UB-I medium is between 0.15-0.3 μM.

In some embodiments, the UB-II medium comprises or is supplemented with one or more, or all of FGF2, TTNPB, and LDN-193189. In some embodiments, the UB-II medium comprises or is supplemented with all of FGF2, TTNPB, and LDN-193189. In some embodiments, the FGF2 for the UB-II medium is at a final concentration of 200 ng/mL or about 200 ng/mL. In some embodiments, the FGF2 for the UB-II medium is between 50-500 ng/mL. In some embodiments, the FGF2 for the UB-II medium is between 100-400 ng/mL. In some embodiments, the FGF2 for the UB-II medium is between 150-300 ng/mL. In some embodiments, the FGF2 for the UB-II medium is between 175-250 ng/mL. In some embodiments, the TTNPB for the UB-II medium is at a final concentration of 0.1 μM or about 0.1 Mm. In some embodiments, the TTNPB for the UB-II medium is between 0.01-0.5 μM. In some embodiments, the TTNPB for the UB-II medium is between 0.05-0.3 μM. In some embodiments, the TTNPB for the UB-II medium is between 0.07-0.2 M. In some embodiments, the TTNPB for the UB-II medium is between 0.08-0.15 M. In some embodiments, the LDN193189 for the UB-II medium is at a final concentration of 30 nM or about 30 nM. In some embodiments, the LDN193189 for the UB-II medium is between 10-100 nM. In some embodiments, the LDN193189 for the UB-II medium is between 15-80 nM. In some embodiments, the LDN193189 for the UB-II medium is between 20-60 nM. In some embodiments, the LDN193189 for the UB-II medium is between 25-40 nM.

Various embodiments provide methods for generating a renal collecting duct (CD) organoid, which includes culturing a renal ureteric bud (UB) organoid in a CD differentiation medium, said CD differentiation medium comprises a basal medium and supplements, thereby generating a CD organoid. In further embodiments, a method for generating a renal collecting duct (CD) organoid includes generating a renal ureteric bud (UB) organoid, and culturing the renal UB organoid in a CD differentiation medium, thereby generating a CD organoid. In various embodiments, these methods are also for differentiating UB cells into a renal collecting duct, or into a CD organoid. In further embodiments, the cultivation in the CD differentiation medium includes cultivation for 7 days or longer to form an elongated CD organoid morphology. In some aspects, the CD organoid is characterized by elevated expressions of a principal cell (PC)-specific marker and/or an intercalated cell (IC)-specific marker. In further aspects, the PC-specific marker comprises one or more of AQP2 and AQP3, and the IC-specific marker comprises one or more of FOXI1, ATP6V1B1, SLC4A1/AE1, and SLC26A4/PENDRIN.

In some embodiments of generating the CD organoid, the UB organoid comprises or is generated with human ureteric bud progenitor cells (UPCs) or human UB cells, and the supplements of the CD differentiation medium comprise one or more, or all of aldosterone, vasopressin, and KNOCKOUT serum replacement (KSR). In some embodiments, the supplements of the CD differentiation medium comprise all of aldosterone, vasopressin, and KSR.

In some embodiments of generating the CD organoid, the UB organoid comprises or is generated with mouse ureteric bud progenitor cells (UPCs) or mouse UB cells, and the supplements of the CD differentiation medium comprises one or more, or all of FGF9, Y27632, DAPT, PD0325901, aldosterone, and vasopressin. In some embodiments, the supplements of the CD differentiation medium comprises all of FGF9, Y27632, DAPT, PD0325901, aldosterone, and vasopressin.

In some embodiments, the aldosterone in the CD differentiation medium for differentiating UPCs or UB cells into CD organoids, (human or mouse sourced,) is at a final concentration of 100 nM or about 100 nM. In some embodiments, the aldosterone in the CD differentiation medium is between 50-60, 60-70, 70-80, 80-90, 90-100, 100-110, 110-120, 120-130, 130-140, or 140-150 nM, or any combination thereof.

In some embodiments, the vasopressin in the CD differentiation medium for differentiating UPCs or UB cells into CD organoids, (human or mouse sourced,) is at a final concentration of 1 IU/mL, or about 1 IU/mL. In some embodiments, the vasopressin in the CD differentiation medium is between 0.5-0.6, 0.6-0.7, 0.7-0.8, 0.8-0.9, 0.9-1.0, 1.0-1.1, 1.1-1.2, 1.2-1.3, 1.3-1.4 or 1.4-1.5 IU/mL, or any combination thereof.

In some embodiments, the KSR in the CD differentiation medium for differentiating human UPCs or human UB cells into CD organoids is at a final concentration of 3% (v/v) or about 3% (v/v). In some embodiments, the KSR in the CD differentiation medium is between 0.5-1, 1-1.5, 1.5-2, 2-2.5, 2.5-3, 3-3.5, 3.5-4, 4-5, 5-6, 6-7, 7-8, or 8-10% (v/v), or any combination thereof. In some embodiments, KSR is not included or supplemented to the CD differentiation medium for differentiating mouse UPCs or mouse UB cells into CD organoids.

In some embodiments, the FGF9 in the CD differentiation medium for differentiating mouse UPCs or mouse UB cells into CD organoids is at a final concentration of 50 ng/mL or about 50 ng/mL. In some embodiments, the FGF9 in the CD differentiation medium is between 10-20, 20-30, 30-40, 40-50, 50-60, 60-70, 70-80, 80-90, or 90-100 ng/mL, or any combination thereof. In some embodiments, FGF9, or any FGF, is not included or supplemented to the CD differentiation medium for differentiating human UPCs or human UB cells into CD organoids.

In some embodiments, the Y27632 in the CD differentiation medium for differentiating mouse UPCs or mouse UB cells into CD organoids is at a final concentration of 10 μM or about 10 μM. In some embodiments, the Y27632 in the CD differentiation medium is between 1-2, 2-3, 3-4, 4-5, 5-6, 6-7, 7-8, 8-9, 9-10, 10-12, 12-14, 14-16, 16-18, or 18-20 μM, or any combination thereof. In some embodiments, Y27632 is not included or supplemented to the CD differentiation medium for differentiating human UPCs or human UB cells into CD organoids.

In some embodiments, the PD0325901 in the CD differentiation medium for differentiating mouse UPCs or mouse UB cells into CD organoids is at a final concentration of 1 μM or about 1 μM. In some embodiments, the PD0325901 in the CD differentiation medium is between 0.1-0.2, 0.2-0.3, 0.3-0.4, 0.4-0.5, 0.5-0.6, 0.6-0.7, 0.7-0.8, 0.8-0.9, 0.9-1.0, 1.0-1.1, 1.1-1.2, 1.2-1.3, 1.3-1.4, 1.4-1.5, 1.5-1.6, 1.6-1.7, 1.7-1.8, 1.8-1.9, or 1.9-2.0 μM, or any combination there of. In some embodiments, PD0325901 is not included or supplemented to the CD differentiation medium for differentiating human UPCs or human UB cells into CD organoids.

In some embodiments, the DAPT in the CD differentiation medium for differentiating mouse UPCs or mouse UB cells into CD organoids is at a final concentration of 5 μM or about 5 M. In some embodiments, the DAPT in the CD differentiation medium is between 1-2, 2-3, 3-4, 4-5, 5-6, 6-7, 7-8, 8-9, or 9-10 μM, or any combination thereof. In some embodiments, DAPT is not included or supplemented to the CD differentiation medium for differentiating human UPCs or human UB cells into CD organoids.

Various embodiments provide methods for generating an engineered kidney, or generating an engineered kidney model in vitro or ex vivo or de novo, which includes combining UB tip cells (or UPC cells, or a tip portion of cells from a branch of an UB organoid) with nephron progenitor cells (NPCs) in one culture, and cultivating the combination in a kidney reconstruction medium, to generate a tubular network with connected nephron-like cell types and a collecting duct.

In some embodiments, the methods for generating an engineered kidney includes inserting the tip portion of the renal UB organoid (or UB tip cells) into an excavated cavity of a culture of the NPCs to obtain a mixture of cells, and cultivating the mixture in an air-liquid interface. In some embodiments, the mixture does not include interstitial progenitor cells.

In some embodiments, the kidney reconstruction medium comprises, or is supplemented with one or both of: TTNPB and Y27632. In some embodiments, the kidney reconstruction medium comprises, or is supplemented with TTNPB and Y27632. In some embodiments, the kidney reconstruction medium comprises APEL2 basal medium and is supplemented with both of TTNPB and Y27632.

In some embodiments, the TTNPB in the reconstruction medium is at a final concentration of 0.1 μM or about 0.1 M. In some embodiments, the TTNPB in the reconstruction medium is between 0.01-0.03, 0.03-0.05, 0.05-0.07, 0.07-0.1, 0.1-0.15, 0.15-0.2, 0.2-0.25, 0.25-0.3, 0.3-0.35, 0.35-0.4 or 0.4-0.5 μM, or any combination thereof.

In some embodiments, the Y27632 in the reconstruction medium is at a final concentration of 10 μM or about 10 μM. In some embodiments, the Y27632 in the reconstruction medium is between 1-2, 2-3, 3-4, 4-5, 5-6, 6-7, 7-8, 8-9, 9-10, 10-12, 12-14, 14-16, 16-18, or 18-20 μM, or any combination thereof.

In some embodiments, the APEL2 is a basal medium used in the reconstruction medium, and APEL2 is a defined, animal component-free medium available at STEMCELL Technologies (catalog no. 05270, 05275).

Various embodiments of the invention provide an ureteric bud (UB) organoid, which can be generated by a method disclosed herein. In some embodiments, an UB organoid is provided, which comprise at least 99%, 98%, 97%, 96%, or 95% of UB progenitor cells that express one or more markers for UPCs, one or more UPC regulators, and/or one or more UB lineage markers, wherein the markers for UPCs comprise RET and WNT11, the UPC regulators comprise RET, ETV5, and SOX9, and the UB lineage markers comprise GATA3, PAX2, KRT8, and CDH1.

Various embodiments of the invention also provide an ureteric bud (UB) organoid for ex vivo modeling of a kidney disease, in which at least a fraction of the cells in the UB organoid comprise at least one edited gene, wherein the edited gene comprises a mutation, an overexpression, a down regulation, a knock out, or a combination thereof.

The UB organoids, CD organoids, engineered kidneys (or kidney organoids) encompassed by the present disclosure can be used in various screening applications. In some examples, UB organoids, CD organoids, engineered kidneys can be used to screen a candidate compound for therapeutic efficacy in treating kidney disease or disorder. In other examples, UB organoids, CD organoid, or kidney organoids can be used to screen for toxicity. For example, kidney organoids can be used to screen for nephrotoxicity.

Various embodiments provide a method of screening for a candidate drug for treating, reducing the incidence or severity of a kidney disease and/or for promoting kidney regeneration, which includes contacting a molecule of interest with an UB organoid generated; and measuring a level of a biomarker transcribed or expressed in the UB organoid before with contact of the molecule of interest, and measuring a level of the biomarker transcribed or expressed in the UB organoid in the presence of the molecule of interest.

Further embodiments provide a method of screening for a candidate drug for treating, reducing the incidence or severity of a kidney disease and/or for promoting kidney regeneration, comprising contacting a molecule of interest with an engineered kidney generated; and measuring a level of a biomarker transcribed or expressed in the engineered kidney before contact of the molecule of interest, and measuring a level of the biomarker transcribed or expressed in the engineered kidney in the presence of the molecule of interest.

In some embodiments, the biomarker is associated with a disease or condition in the renal system, e.g., having an elevated expression level or transcription level in a subject with a disease or condition in the renal system, compared to a reference from a subject who does not have the disease or condition in the renal system. In some embodiments of the screening methods, a level of the biomarker in the presence of the molecule of interest below that before the contact with the molecule of interest is indicative that the molecule of interest is a candidate agent or is likely to inhibit, reduce the severity, or treat the disease or condition in the renal system. In some embodiments, a level of the biomarker in the presence of the molecule of interest above that before the contact with the molecule of interest is indicative that the molecule of interest is not a candidate agent or is not likely to inhibit, reduce the severity, or treat the disease or condition in the renal system.

In some embodiments, kidney organoids (UB organoids, CD organoids, or engineered kidney disclosed herein) are representative of a kidney disease, which can be assessed to screen for therapeutic efficacy. For example, the kidney disease can be selected from the group consisting of congenital nephrotic syndrome (CNS) including steroid resistant nephrotic syndrome and Finnish nephropathy, focal segmental glomerulonephritis (FSGS), Alport syndrome and Pierson syndrome. In another example, the kidney disease is polycystic kidney disease.

Further embodiments provide a method of screening a candidate compound for nephrotoxicity, which includes contacting a kidney organoid (UB organoid, CD organoid, or engineered kidney) disclosed herein with a candidate compound and measuring or assessing for nephrotoxic side effects, so as to determine whether or not the candidate compound is nephrotoxic.

Exemplary nephrotoxic side effects include direct tubular effects, podocyte injury, interstitial nephritis and glomerulonephritis. Nephrotoxicity can also be assessed or measured by an appropriate test for kidney cell function in vitro, including analysis of biomarker expression using commercially available tools including, for example, the Human Nephrotoxicity RT2 PROFILER™ PCR Array from Qiagen or the High Content Analysis (HCA) Multiplexed Nephrotoxicity Assay from Eurofins. In other examples, nephrotoxicity is assessed by measuring acute apoptosis of glomerular cells following contact with a candidate compound; using electron microscopy such as transmission EM or scanning EM. Other examples indicative of nephrotoxicity include loss of podocyte marker gene expression or protein expression and loss of foot processes (loss of effacement).

A set of supplements is also provided for cultivating human ureteric bud (UB) progenitor cells in a medium to generate UB organoids, which comprises LDN-193189, TTNPB, CHIR99021, Janus-associated kinase inhibitor I (JAK inhibitor I), glial cell-derived neurotrophic factor (GDNF), A83-01, R-spondin 1, fibroblast growth factor (FGF) 7, SB202190, and epidermal growth factor (EGF).

In some embodiments, the set of supplements for cultivating human ureteric bud (UB) progenitor cells in a medium to generate UB organoids does not comprises Y27632.

A medium composition is also provided for cultivating human ureteric bud (UB) progenitor cells to generate UB organoids, which comprises a basal medium, L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution, and the set of supplements of LDN-193189, TTNPB, CHIR99021, Janus-associated kinase inhibitor I (JAK inhibitor I), glial cell-derived neurotrophic factor (GDNF), A83-01, R-spondin 1, fibroblast growth factor (FGF) 7, SB202190, and epidermal growth factor (EGF).

In further embodiments, a set of supplements is also provided for cultivating mouse ureteric bud (UB) progenitor cells in a medium to generate UB organoids, which comprises FGF9, TTNPB, CHIR99021, GDNF, LDN-193189, A83-01, JAK Inhibitor I, SB202190, and R-Spondin 1, said supplements do not comprise EGF.

In some embodiments, a medium composition is provided for cultivating mouse ureteric bud (UB) progenitor cells to generate UB organoids, which comprises a basal medium, L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution, and the set of supplements of FGF9, TTNPB, CHIR99021, GDNF, LDN-193189, A83-01, JAK Inhibitor I, SB202190, and R-Spondin 1.

A medium composition is provided for differentiating human ureteric bud (UB) organoids, or human UB progenitor cells, into a renal collecting duct (CD) organoid, comprising a basal medium and supplements of aldosterone, vasopressin, and KNOCKOUT serum replacement (KSR), and L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution.

A medium composition is further provided for differentiating mouse ureteric bud (UB) organoids, or mouse UB progenitor cells, into a renal collecting duct (CD) organoid, comprising a basal medium and supplements of FGF9, Y27632, DAPT, PD0325901, aldosterone, and vasopressin, and L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution.

A kit or assay for use in screening applications is also provided. For example, a kit or assay is for use in screening candidate compounds for nephrotoxicity and/or therapeutic efficacy. In some embodiments, UB organoids, CD organoids, or engineered kidneys are provided in culture, and candidate compounds can then be contacted therewith and screened for nephrotoxicity and/or therapeutic efficacy. Accordingly, in some embodiments, an assay is provided when used for screening, the assay comprising UB organoids, CD organoids, or engineered kidneys disclosed herein in culture. In some embodiments, UB organoids, CD organoids, or engineered kidneys are provided with culture media or other components for maintaining the organoids in culture. In some embodiments, the UB organoids, the CD organoids, and/or the engineered kidneys are provided with written instructions for performing the methods of the present disclosure. In some embodiments, the assay comprises more than one UB organoid, one CD organoid, or one engineered kidney. For example, the assay can comprise 10, 20, 30 or more UB organoids, CD organoids, and/or engineered kidneys. The UB organoids, the CD organoids, and/or the engineered kidneys can be provided in a single or multi-well format such as a 96 well plate.

In various embodiments of a cell cultivation method and/or a medium composition disclosed herein, a basal medium is supplemented with one or more, or all of L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution; and the basal medium comprises DMEM, or DMEM/F12 (1:1).

### EXAMPLES

### Example 1. Generation of Patterned Kidney Organoid that Recapitulates Adult Kidney Collecting System from Expandable Ureteric Bud Progenitors

Despite previous efforts towards the expansion or de novo generation of the immature UB relying on primary mouse/rat tissue, mouse embryonic stem cells or human pluripotent stem cells, we still lack a robust kidney organoid model that can generate and expand the UB progenitor cells, and recapitulate the maturation and spatial patterning of the adult CD.

Expanding Mouse UB Progenitor Cells into 3D Branching UB Organoids

We previously developed a 3D culture system for the long-term expansion of mouse and human nephron progenitor cells (NPCs), which can generate nephron organoids that recapitulate kidney development and disease. UB branching morphogenesis is driven by another kidney progenitor population, the UB progenitor cells (UPCs). UPCs are specified around embryonic day 10.5 (E10.5), when the UB starts to invade the MM. UPCs disappear around postnatal day 2 (P2), when nephrogenesis ceases. Self-renewing UPCs reside in the tip region of the branching UB. During their approximately 10-day lifespan, some UPCs migrate out of UB tip niche to the UB trunk, and differentiate into the renal CD network. Other UPCs proliferate and replenish the self-renewing progenitor cell population of the UB tip. Ret and Wnt11 have been identified as specific markers for UPCs and regulate UPC programs directly (Ret) or through feedback mechanisms (Wnt11). A transgenic reporter mouse strain Wnt11-myrTagRFP-IRES-CE (“Wnt11-RFP” for short) facilitates the real-time tracking of Wnt11-expressing cells based on RFP expression, and the lineage tracing of their progeny via a Cre-mediated recombination system.

We employed this Wnt11-RFP reporter system as a readout to screen for a culture condition that maintained the progenitor identity of UPCs in vitro. T-shaped UBs were manually isolated from E11.5 kidneys of Wnt11-RFP mice, and immediately embedded into Matrigel to set up a 3D culture platform that supported epithelial branching. In this 3D culture format, built on previous efforts towards the ex vivo culture of UB, hundreds of different combinations of growth factors and small molecules were tested (see also the Materials and Techniques section below for details under subheading “Screening for optimal UB culture condition”). A brief summary of this screening workflow:
Stage I. Using Wnt11-RFP, starting from Yuri et al., Stem Cell Reports, 8, 401-416, (2017)
   Branching morphogenesis confirmed
   Limited expansion and quick loss of Wnt11-RFP
Stage II: Improvement of individual components from Yuri et al., Stem Cell Reports, 8, 401-416, (2017)
   Is every component (FGF, RA, C1, GDNF) needed?→Yes
   Improvement: RA→TTNPB
   Improvement: FGF1→FGF9
   Improvement: CA→C3 (CHIR99021 at 3 μM)
Stage III: Screening of new growth factors and chemicals
   Base condition (FGF9, TTNPB, C3, GDNF)
   1^{st} round screening for individual hits
   2^{nd} round screening for combinatorial effects
   UBCM identified (FGF9, TTNPB, C3, GDNF, LDN, A83, JAKI, SB, Rspo1)
Stage IV: Validation of UBCM
   Is every component needed in the UBCM? →Yes

TABLE 1
Summary of the 1^{st} round screening in Stage III for identifying individual hits.

| | | | | Organoid | Selected for |
|---|---|---|---|---|---|
| Reagent Name | Concentration | | Wnt11-RFP | growth | R2 screening |
| CHIR99021 | 6 | μM | — | — | — |
| FGF2 | 200 | ng/ml | Worse | — | — |
| FGF4 | 50 | ng/ml | Worse | — | — |
| FGF7 | 50 | ng/ml | — | — | Y |
| FGF8 | 100 | ng/ml | Worse | — | — |
| FGF10 | 100 | ng/ml | Worse | Slower | — |
| FGF20 | 50 | ng/ml | Worse | — | — |
| Activin A | 20 | ng/ml | Worse | Slower | — |
| A83-01 | 200 | nM | Better | — | Y |
| BMP7 | 10 | ng/ml | Better | — | Y |
| LDN193189 | 100 | nM | Better | — | Y |
| Purmorphamine | 1 | μM | — | — | — |
| KAAD-Cyclopamine | 100 | nM | — | — | — |
| JAG-1 | 1 | μM | — | — | — |
| DAPT | 200 | nM | Better | — | Y |
| SP600126 | 10 | μM | — | — | — |
| S6202190 | 5 | μM | Slightly Better | — | Y |
| PD0325901 | 1 | μM | Dead at D 6 | No growth | — |
| VEGF | 50 | ng/ml | — | — | — |
| Y27632 | 10 | μM | — | — | — |
| R-Spondin 1 | 100 | ng/ml | Better | — | Y |
| Heparin | 1 | μg/ml | Slightly Better | — | Y |
| SCF | 50 | ng/ml | Better | Slightly better | Y |
| KSR | 15% | | Better | — | Y |
| LY294002 | 5 | μM | Better | — | Y |
| Cyclosporine | 10 | μM | Better | — | Y |
| TNF-α | 100 | ng/m1 | Worse | — | — |
| HGF | 50 | ng/m1 | — | — | — |
| EGF | 50 | ng/m1 | Better | — | Y |
| Forskolin | 10 | μM | Worse | — | — |
| LIE | 10³ | units/ml | — | — | — |
| JAKI | 100 | nM | Slightly Better | — | Y |
| 1GF1 | 20 | ng/m1 | — | — | — |
| 1GF2 | 2 | ng/ml | — | — | — |
| AICAR | 0.5 | mM | Slighty Better | — | Y |
| Melformin | 1 | mM | Slightly Better | — | Y |
| Abbott | 0.1 | mM | — | — | — |
| XMU-MP-1 | 1 | μM | — | No growth | — |
| Verteporfin | 1 | μM | — | Dead | — |
| PDGF-BB | 10 | ng/m1 | — | — | — |

In the columns of “Wnt11-RFP” and “Organoid growth”: “—” indicates no significant differences were observed compared to the control group (FGF9 + C3 + TTNPB + GDNF). In the column of “Selected for R2 screening”: “Y” indicates the factor was selected for 2^{nd} round (R2) screening; “—” indicates the factor was not selected.

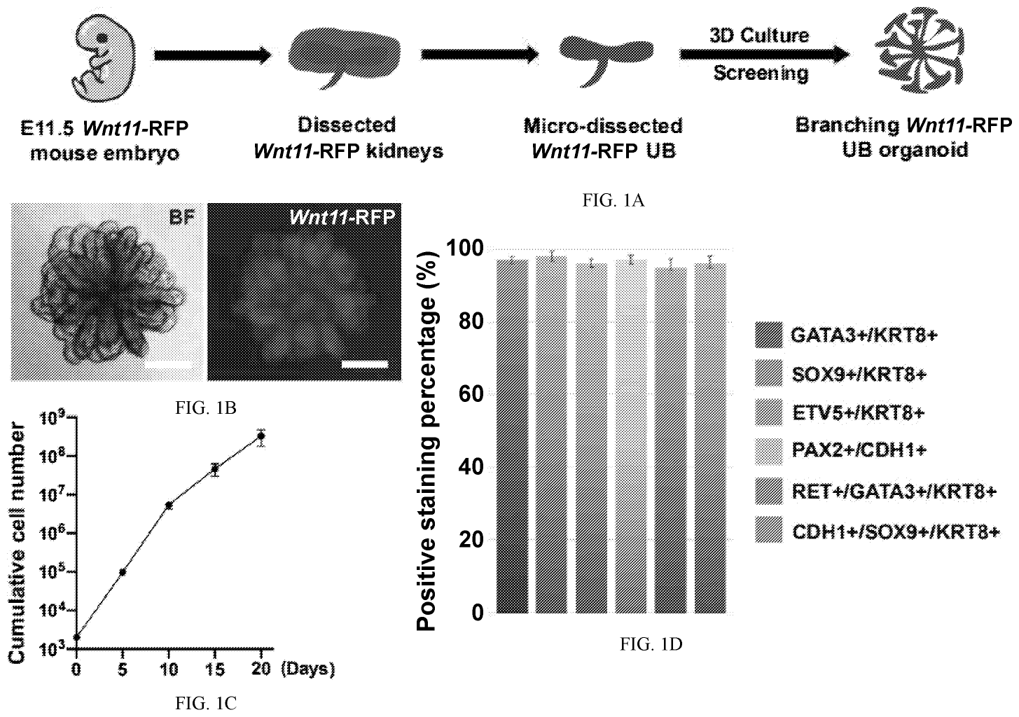

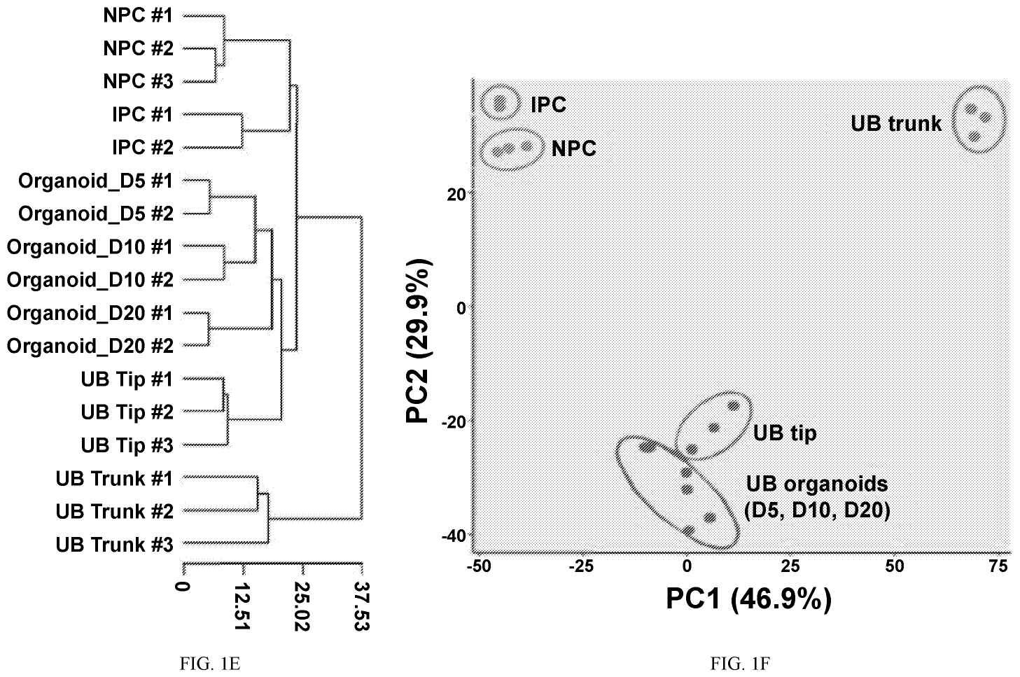

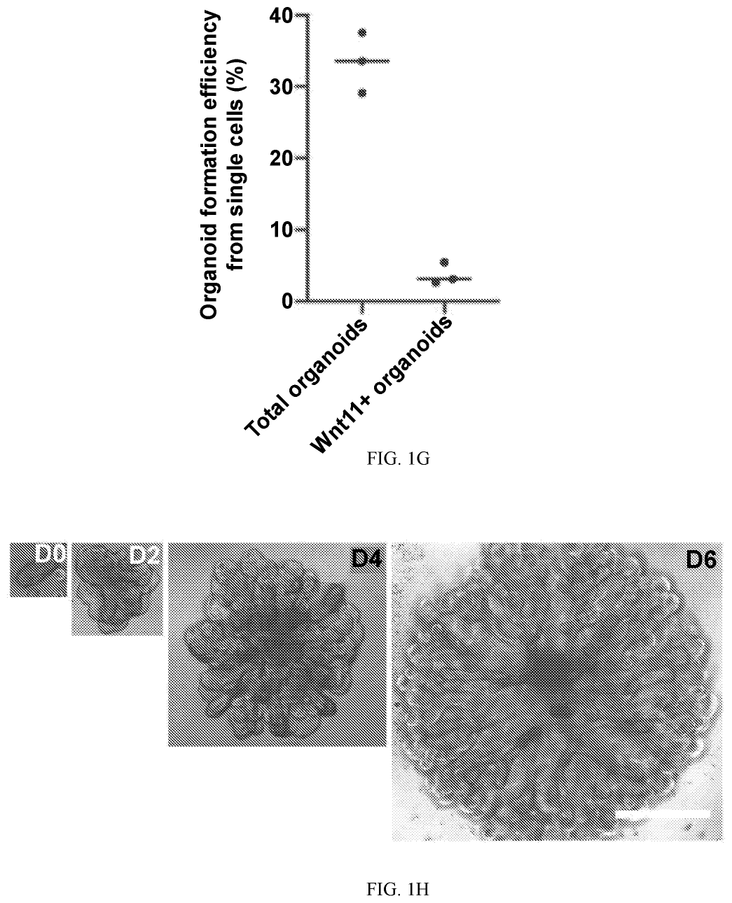

This screening allowed us to identify a cocktail, which we named “UB culture medium” (UBCM, Table 2), that maintained self-renewing UPCs as a 3D branching UB organoid (FIG. 1A).

TABLE 2
Medium recipe of mUBCM (mouse UB culture medium),
as supplements to base medium DMEM/F12 (1:1) (1×),
Invitrogen #11330-032.

| | | | Final | |
|---|---|---|---|---|
| Reagent Name | Company | Cat. No. | Concentration | |
| GlutaMAX-I (100×) | Invitrogen | 35050-079 | 1× | |
| MEM NEAA (100×) | Invitrogen | 11140-050 | 1× | |
| 2-Mercaptoethanol | Invitrogen | 21985-023 | 0.1 | mM |
| Pen Strep (100×) | Invitrogen | 15140-122 | 1× | |
| B-27, minus vitamin A | Invitrogen | 12587-010 | 1× | |
| ITS (100×) | Sigma | I3146-5ML | 1× | |
| LDN-193189 | Reagents | 36-F52 | 200 | nM |
| | Direct | | | |
| TTNPB | TOCRIS | 0761 | 0.1 | μM |
| CHIR99021 | Reagents | 27-H76 | 3 | μM |
| | Direct | | | |
| JAK Inhibitor I | Stemcell | 74022 | 100 | nM |
| | Technologies | | | |
| GDNF (mouse) | PeproTech | 450-44-50 μg | 50 | ng/ml |
| A83-01 | STEMGENT | 04-0014 | 0.2 | μM |
| Rspo1 | R&D Systems | 4645-RS-100 | 100 | ng/ml |
| FGF9 | R&D Systems | 273-F9-025 | 50 | ng/ml |
| SB202190 | Axon | Axon 1304 | 5 | μM |
| | Medchem | | | |

Under this culture condition, the T-shaped UB formed a rapidly expanding branching epithelial morphology. More importantly, in contrast to prior UB culture system that generated a mixture of both UB tip and trunk cell types, uniform Wnt11-RFP expression was maintained throughout the 3D structure in the UBCM-derived UB organoid, indicating the capture of a relatively pure UPC population (FIG. 1B). Resected Wnt11-RFP⁺ UB organoid tips, re-embedded in Matrigel, branched and grew into additional Wnt11-RFP⁺ UB organoids. Repetitive passaging and embedding for up to 3 weeks, resulted in more than a hundred thousand-fold expansion in the number of cells (FIG. 1C). Wnt11-RFP levels remained uniform for the first 10 days but progressively dropped thereafter, similar to the normal time course of UPCs in vivo. Consistent with the uniform expression of Wnt11-RFP throughout UB organoids at 10 days, whole-mount immunostaining confirmed the homogenous expression of the critical UPC regulators Ret, Etv5, and Sox9, as well as broad UB lineage markers Gata3, Pax2, Krt8, and Cdh1 (FIG. 1D).

To better define the identity of the UB organoids, we used RNA-seq to profile the transcriptome of the organoids after 5 days, 10 days and 20 days in culture. These data were compared with prior RNA-seq data for primary UB tip and UB trunk populations, as well as for NPCs and interstitial progenitor cells (IPCs). Unsupervised clustering (FIG. 1E) and principal component analysis (PCA) (FIG. 1F) placed the cultured UB organoids closer to the primary UB tip samples than to differentiated stalk derivatives of the UB trunk. Taken together, these findings indicate the UB organoid culture system enables a substantial expansion of cells retaining molecular characteristics of UPCs in vitro.

Next, we tested whether the UB organoid culture system could be applied to mouse strains other than Wnt11-RFP. For this, we successfully derived UB organoids from E11.5 UB from Swiss Webster, a random-bred laboratory mouse strain, and from multiple transgenic strains including Hoxb7-Venus, Sox9-GFP, and Rosa26-Cas9/GFP. All of these UB organoids retained the typical branching morphology and showed very similar growth rates, compared to Wnt11-RFP UB organoids (Table 3), indicating the robustness of the 3D/UBCM culture system. Importantly, UB organoids self-organized into branching organoids after a freeze-thaw cycle, enabling cryostorage and reseeding of UB cultures.

TABLE 3
Summary of UB organoid derivation from mouse strains with
different genetic backgrounds or with different derivation methods.

| Genetic back- | | | | Wnt11-RFP | Hoxb7- | | Rosa26- | |
|---|---|---|---|---|---|---|---|---|
| ground of the UB | Swiss- | Wnt11- | Wnt11- | (freeze & | Ve- | Sox9- | Cas9/ | Wnt11- |
| Organoid | Webster | RFP | RFP | thaw) | nus | GFP | GFP | RFP |
| Starting | Intact T- | Intact T- | Intact T- | Intact T-shaped | Intact T- | Intact T- | Intact T- | Single cells |
| Materials | shaped | shaped | shaped | UB | shaped UB | shaped UB | shaped UB | of E11.5 UB |
| | UB | UB | UB | | | | | |
| Passage Method | Manual | Manual | Single | Single Cell | Manual | Manual | Single Cell | Single Cell |
| | | | Cell | | | | | |
| Typical Passage | 5 | 5 | 5 | 5 | 5 | 5 | 5 | 5 |
| Cycle (Days) | | | | | | | | |
| Passage # | P2-P3 | P2~P3 | P2~P3 | P2~P3 | P2~P3 | P2~P3 | P2~P3 | P2~P3 |
| Cultured Time | 15-20 | 15-20 | 15-20 | 15-20 | 15-20 | 15-20 | 15-20 | 11 |
| (Days) | | | | | | | | |
| Maintained Re- | N/A | ~15 | ~15 | ~15 | >20 | >20 | >20 | 9-10 |
| porter Expression | | | | | | | | |
| (Days) | | | | | | | | |
| Fold of UB | 10⁴~10⁵ | 10⁴~10⁵ | 10⁴~10⁵ | 10⁴~10⁵ | 10⁴~10⁵ | 10⁴~10⁵ | 10⁴~10⁵ | 10³~10⁴ |
| expansion | | | | | | | | |
| Doublings | 0.865-0.936 | 0.865-0.936 | 0.865-0.936 | 0.865-0.936 | 0.865-0.936 | 0.865-0.936 | 0.865-0.936 | 0.865-0.936 |
| per day | | | | | | | | |

To determine whether UBCM culture conditions enabled clonal growth from a single UPC, dissociated E11.5 UBs were embedded at clonal density in Matrigel and cultured in UBCM medium. Around 30% of the single cells self-organized into E11.5 UB-like budding structures within 5 days, though a smaller percentage (3-5%) maintained Wnt11-RFP (FIG. 1G), an efficiency similar to clonal organoid formation for Lgr5⁺ intestinal stem cells. Importantly, the clonally-derived Wnt11-RFP⁺ budding structures were identical to intact E11.5 UB-derived organoids in both branching morphology and growth rate (Table 3, FIG. 1H). Furthermore, withdrawal of the major medium components from UBCM resulted in either growth arrest (CHIR99021 and GDNF) or rapid loss of Wnt11-RFP (all components except for CHIR99021), indicating that each component was essential for optimal UB organoid culture. These data, taken together, indicate that UBCM represents a synthetic niche for the in vitro expansion of UPCs.

Screening for Conditions to Mature UB Organoids into CD Organoids

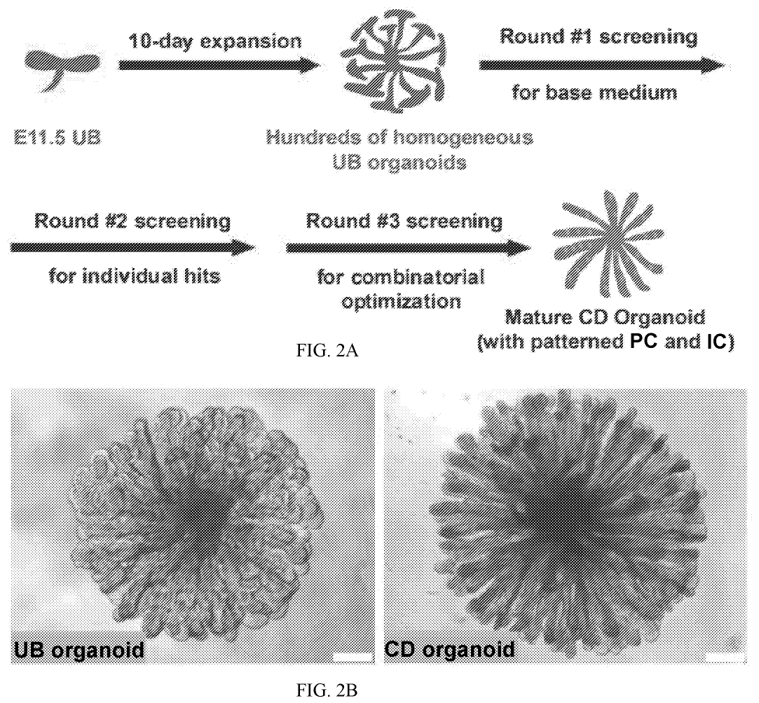

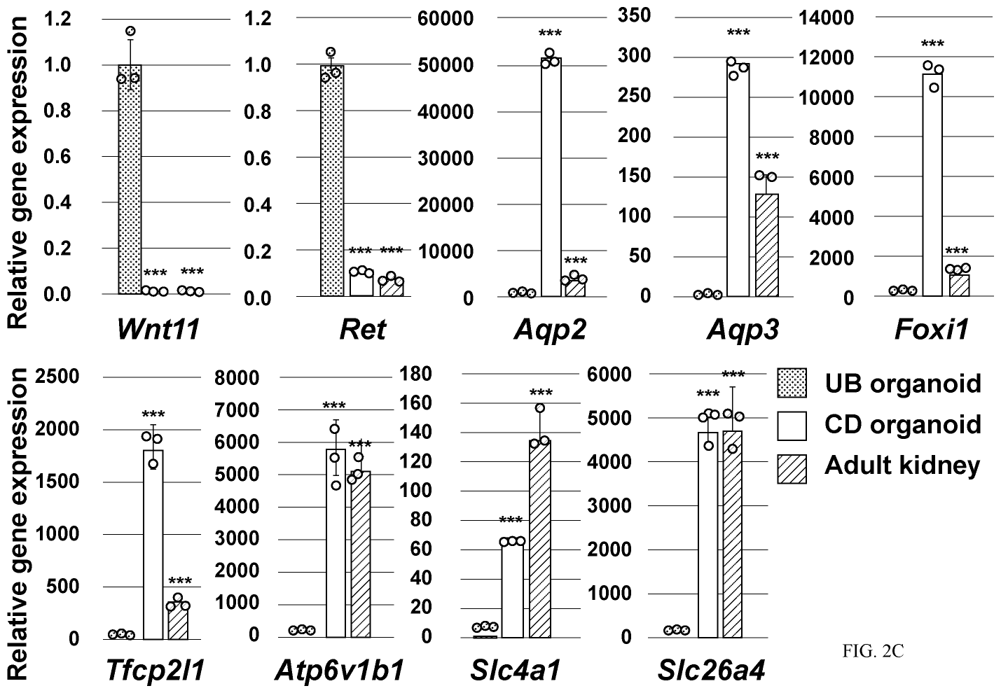

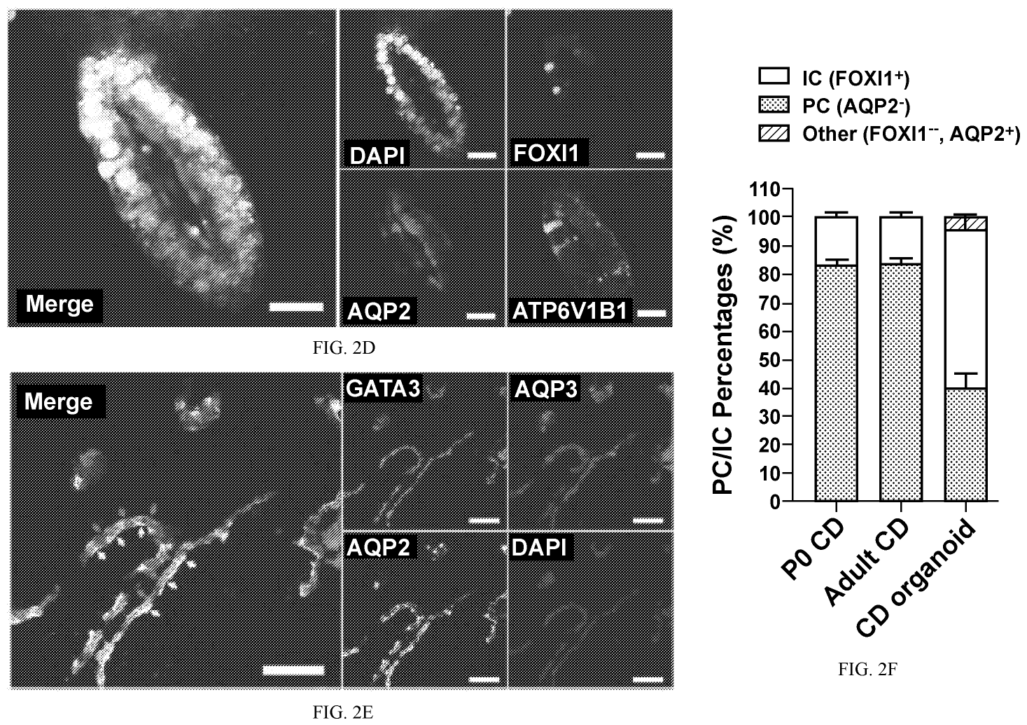

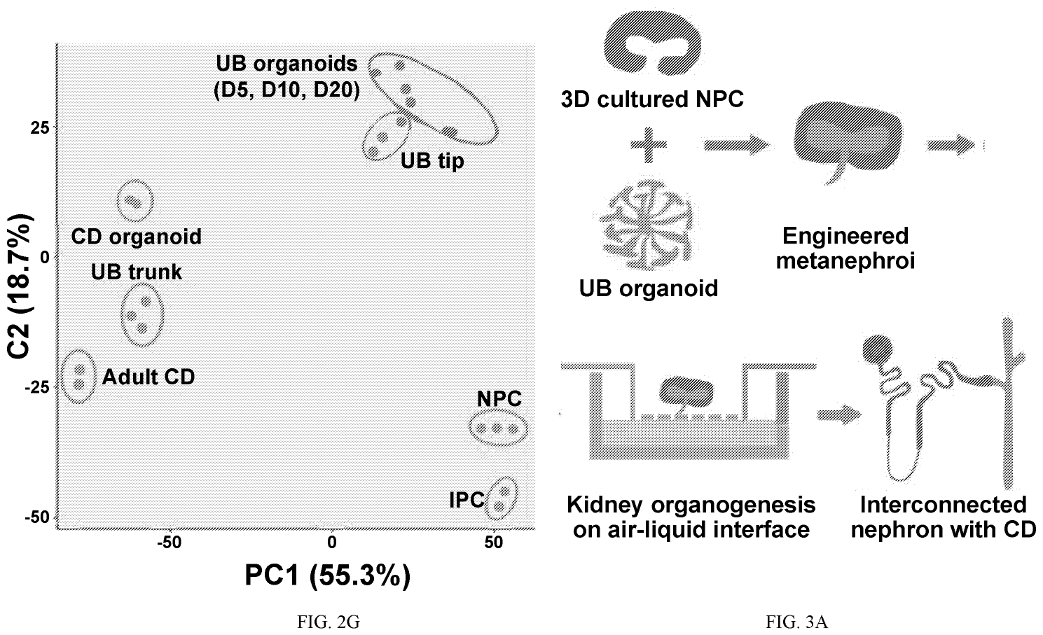

The functions of the mature renal CD system are carried out by two major cell populations that are intermingled throughout the entire CD network. The more abundant principal cells (PCs) concentrate the urine and regulate Na⁺/K⁺ homeostasis via water and Na⁺/K⁺ transporters. The less abundant α- and β-intercalated cells (ICs) regulate normal acid-base homeostasis via secretion of H⁺ or HCO₃⁻ into the urine. The absence of an in vitro system recapitulating PC and IC development in an appropriate 3D context, constrains physiological exploration, disease modeling and drug screening on the renal CD system. With this limitation in mind, we developed a screen to establish conditions supporting the differentiation of CD organoids, assaying expression of Aqp2 and Foxi1, definitive markers for PC and IC lineages, respectively, by quantitative reverse transcription PCR (qRT-PCR), following 7 days of culture under variable but defined culture conditions (FIG. 2A).

In a 1^{st} round of screening, we determined the base condition in which minimal growth factors/small molecules sustained the survival of the organoids and permitted their differentiation. The base medium used for UBCM-Hbi (Li Z, et al., Cell Stem Cell, 19, 4, 516-529, 2016)—was tested, together with the commercially available APEL medium for sustaining kidney organoid generation. Combinations of FGF9, EGF and Y27632 were tested, together with the two different base media (Table 4).

TABLE 4
Summary of conditions tested in the 1st round
of CD differentiation condition screening.

| Culture Conditions | Base Medium | FGF9 | EGF | Y27632 |
|---|---|---|---|---|
| R1-1 | hBI | + | − | − |
| R1-2 | hBI | + | − | + |
| R1-3 | hBI | + | + | − |
| R1-4 | hBI | + | + | + |
| R1-5 | APEL | + | − | − |
| R1-6 | APEL | + | − | + |
| R1-7 | APEL | + | + | − |
| R1-8 | APEL | + | + | + |

After 7 days of differentiation in the various conditions, we observed that the hBI+FGF9+Y27632 condition enabled the survival of organoids and permitted spontaneous basal differentiation, as assayed by a modest induction of both principal cell (Aqp2) and intercalated cell (Foxi1) specific gene expression from qRT-PCR analyses of the 1^{st} round of CD differentiation condition screening for these two PC marker genes.

To enhance the efficiency of differentiation, we carried out a 2^{nd} round of screening identifying molecules that strongly induced the expression of Aqp2 and/or Foxi1 under the hBI+FGF9+Y27632 condition. Agonists or antagonists targeting major developmental pathways (e.g. TGF-β, BMP, Wnt, FGF, Hedgehog and Notch) were tested, together with hormonal inputs known to regulate PC or IC activity (aldosterone and vasopressin). BMP7, DAPT (a Notch pathway inhibitor), JAKI (JAK inhibitor I) and PD0325901 (MEK inhibitor) dramatically increased both Aqp2 and Foxi1 expression, while JAG-1 (Notch agonist) and aldosterone led to a preferential increase in Foxi1 expression, and vasopressin to enhanced Aqp2 expression (Table 5).

TABLE 5
Summary of chemicals tested in the 2^{nd} round of CD differentiation
condition screening with the R1-2 medium as base medium.

| | Base | | Selected for | |
|---|---|---|---|---|
| Conditions | Condition | Chemical Tested | R3 screen | Note |
| R2-1 | R1-2 | Control | — | |
| R2-2 | R1-2 | +activin A | — | Dramatic |
| | | | | cell death |
| R2-3 | R1-2 | +A83-01 | — | |
| R2-4 | R1-2 | +BMP4 | — | |
| R2-5 | R1-2 | +BMP7 | Y | |
| R2-6 | R1-2 | +LDN193189 | — | |
| R2-7 | R1-2 | +Purmorphamine | — | |
| R2-8 | R1-2 | +KAAD-Cyclopamine | — | |
| R2-9 | R1-2 | +JAG-1 | Y | |
| R2-10 | R1-2 | +DAPT | Y | |
| R2-11 | R1-2 | +CHIR99021 | — | |
| R2-12 | R1-2 | +IWR-1 | — | |
| R2-13 | R1-2 | +TTNPB | — | |
| R2-14 | R1-2 | +LE135 | — | |
| R2-15 | R1-2 | +LIF | — | |
| R2-16 | R1-2 | +JAKI | Y | |
| R2-17 | R1-2 | +FGF1 | — | |
| R2-18 | R1-2 | +FGF2 | — | |
| R2-19 | R1-2 | +FGF7 | — | |
| R2-20 | R1-2 | +FGF10 | — | |
| R2-21 | R1-2 | +SP600126 | — | Dramatic |
| | | | | cell death |
| R2-22 | R1-2 | +SB202190 | — | |
| R2-23 | R1-2 | +PD0325901 | Y | |
| R2-24 | R1-2 | +Aldosterone | Y | |
| R2-25 | R1-2 | +Vasopressin | Y | |

In a 3^{rd} round of screening, testing of various combinations of these factors led to the identification of an optimized CD differentiation medium (CDDM, Table 6 for mouse CDDM, Table 7 for human CDDM) supplemented with FGF9, Y27632, DAPT, PD0325901, aldosterone and vasopressin.

TABLE 6
Medium recipe of mouse CD differentiation medium
(mCDDM), lising supplements to a basal medium: DMEM/F12
(1:1) (1×), Invitrogen, Cat. No. 11330-032.

| | | | Final | |
|---|---|---|---|---|
| | | | Concen- | |
| Reagent Name | Company | Cat. No. | tration | |
| GlutaMAX-I (100×) | Invitrogen | 35050-079 | 1× | |
| MEM NEAA (100×) | Invitrogen | 11140-050 | 1× | |
| 2-Mercaptoethanol | Invitrogen | 21985-023 | 0.1 | mM |
| Pen Strep (100×) | Invitrogen | 15140-122 | 1× | |
| B-27, minus vitamin A | Invitrogen | 12587-010 | 1× | |
| ITS (100×) | Sigma | I3146-5ML | 1× | |
| FGF9 | R&D Systems | 273-F9-025 | 50 | ng/ml |
| Y27632 | Cayman | 10005583 | 10 | μM |
| | Chemical | | | |
| Aldosterone | Sigma-Aldrich | A9477 | 100 | nM |
| Vasopressin | Sigma-Aldrich | V0377-100IU | 1 | I.U./ml |
| PD0325901 | Reagents | 39-C68 | 1 | μM |
| | Direct | | | |
| DAPT | Sigma-Aldrich | D5942 | 5 | μM |

TABLE 7
Medium recipe of human CD differentiation medium
(hCDDM), lising supplements to a basal medium: DMEM/F12
(1:1) (1×), Invitrogen, Cat. No. 11330-032.

| | | | Final | |
|---|---|---|---|---|
| Reagent Name | Company | Cat. No. | Concentration | |
| GlutaMAX-I (100×) | Invitrogen | 35050-079 | 1× | |
| MEM NEAA (100×) | Invitrogen | 11140-050 | 1× | |
| 2-Mercaptoethanol | Invitrogen | 21985-023 | 0.1 | mM |
| Pen Strep (100×) | Invitrogen | 15140-122 | 1× | |
| B-27, minus vitamin A | Invitrogen | 12587-010 | 1× | |
| ITS (100×) | Sigma | I3146-5ML | 1× | |
| Aldosterone | Sigma-Aldrich | A9477 | 100 | nM |
| Vasopressin | Sigma-Aldrich | V0377-100IU | 1 | I.U./ml |

| | | | |
|---|---|---|---|
| KSR | Thermo Fisher | 10828028 | 3% (v/v) |

Generating Mature and Highly Organized CD Organoids from UB Organoids

Seven days of UB organoid culture in CDDM resulted in a morphologically elongated CD organoid phenotype (FIG. 2B). qRT-PCR revealed a marked decrease in the expression of the UPC genes (Wnt11 and Ret) and a concomitant elevation in the expression of PC-specific water transporter encoding genes (Aqp2 and Aqp3) and IC-specific transcription factor (Foxi1), proton pump (Atp6v1b1) and Cl⁻/HCO3⁻ exchangers (Slc4a1/Ae1, α-IC-specific; Slc26a4/Pendrin, β-IC specific) (FIG. 2C). Immunostaining confirmed the presence of AQP2, AQP3, FOXI1, TFCP2L1 and ATP6V1B1 in the CD organoids. Differentiating CD organoids displayed a clear lumen (FIG. 2D), and the organization of PC and IC cell types reflected that of the postnatal mouse kidney CD in which FOXI1⁺/ATP6V1B1⁺/TFCP2L1⁺/KIT⁺ ICs were dispersed in AQP2⁺/AQP3⁺ PCs. Further, in some areas, AQP2 and AQP3 showed a differential subcellular localization, AQP2 to the apical luminal facing surface and AQP3 to basolateral plasma membrane (FIG. 2E), reflecting the normal cellular distribution of these critical components of water trafficking through PC cells. PC and IC ratios in the CD organoids indicated a higher IC portion (50-55%) over PC portion (40-45%) than observed in the neonatal and adult kidney, a likely reflection of DAPT-mediated Notch inhibition in CDDM culture (FIG. 2F); in vivo, IC-derived Notch ligand signaling inhibits the IC fate.

To better define the identity of the CD organoids, we used RNA-seq to profile the transcriptome of the organoids. These data were compared with mouse CD freshly isolated by FACS from the kidney of adult Hoxb7-Venus mice, as well as UB organoids, and prior RNA-seq data for primary UB tip and UB trunk populations, NPCs and IPCs. Principal component analysis (PCA) showed a clear separating of CD organoids from the immature UB tip and UB organoid populations, and similar grouping to UB trunk and primary mouse CD (FIG. 2G), supporting an expected cell maturation of CD organoids. Importantly, the CDDM differentiation protocol was highly reproducible when testing UB organoids derived from different genetic backgrounds (Table 8). The evidence above support that we have, for the first time, generated a mature and patterned kidney organoid that recapitulates the adult kidney collecting system, in a chemically-defined manner.

TABLE 8
Summary of CD organoid derivation efficiency.
All data are presented as mean ± s.d.

| Genetic background of | | | Rosa26- |
|---|---|---|---|
| the UB Organoids | Wnt11-RFP | Wnt11-RFP | Cas9/GFP |
| Starting Materials | Intact T | Intact T | Intact T |
| | shaped UB | shaped UB | shaped UB |
| Passage Method | Manual | Single Cell | Single Cell |
| # of CD induction Tested | 97 | 71 | 27 |
| # of Successful CD induction | 97 | 71 | 27 |
| Differentiation Competency | 100% | 100% | 100% |

Generating Engineered Kidney from Expandable NPCs and UBs

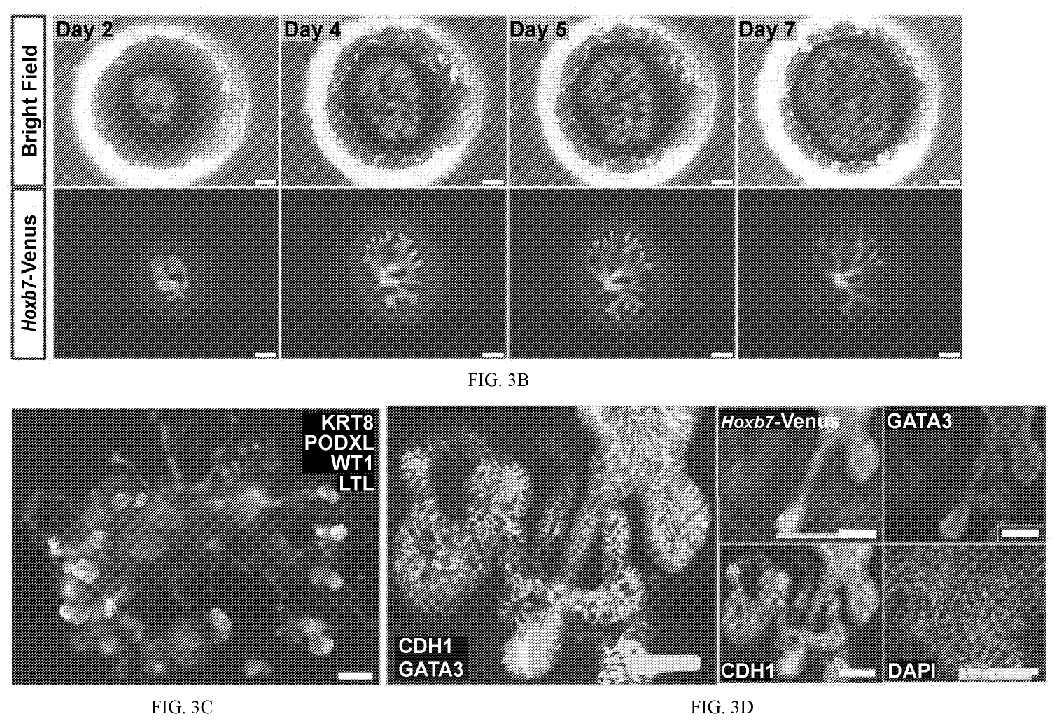

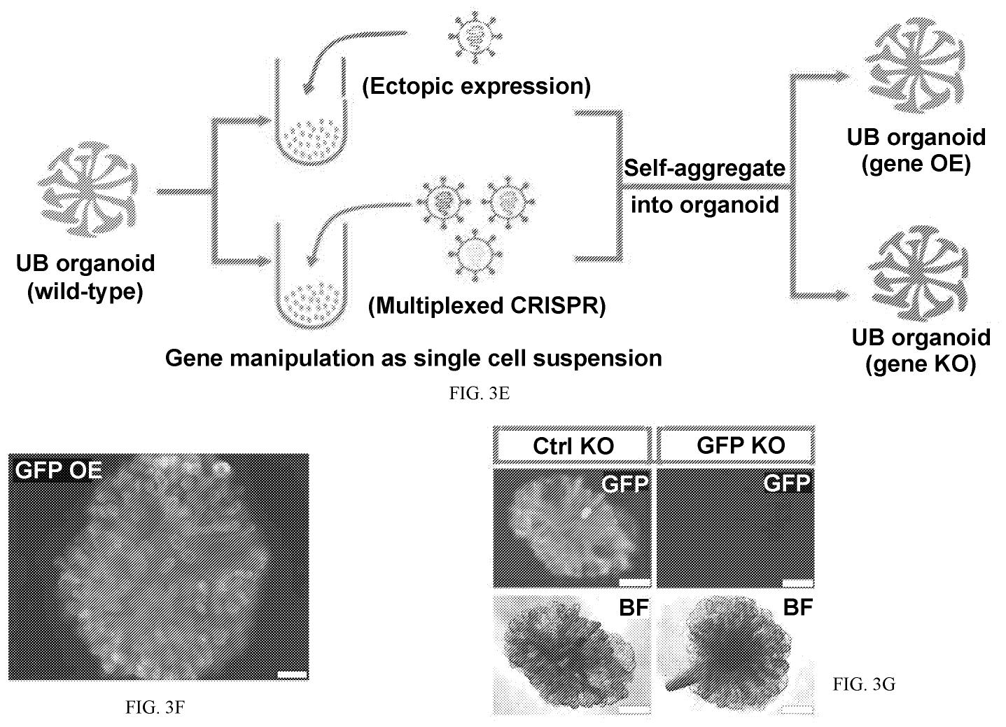

The availability of expandable NPCs and UPCs provides the scalable building blocks required for making a kidney. As a proof-of-concept, we examined whether combining these cell types could generate a model mimicking key features of in vivo kidney development, such as reiterative ureteric branching and nephron induction, and morphogenesis and patterning of differentiating derivatives (FIG. 3A).

NPCs in our long-term culture model grow as 3D aggregates. To mimic the natural organization of NPCs capping UB tips in the kidney anlagen, we manually excavated a cavity in 3D cultured NPCs (expanded several billion fold over for 6-12 months of culture) and inserted a cultured UB organoid tip. The engineered kidney structures were transferred onto an air-liquid interface (ALI) to facilitate further kidney organogenesis. Over 7 days of culture, the inserted Hoxb7-Venus UB organoid tip underwent extensive branching (FIG. 3B) generating a tubular network extending from the center of the structure to the periphery. Further, NPCs generated nephron-like cell types including PODXL⁺/WT1⁺ podocytes and LTL⁺ proximal tubules (FIG. 3C).

To determine whether the engineered kidney also formed a connection between nephron and CD, we engineered kidneys comprising Hoxb7-Venus UB and wild-type NPCs. In this way, all progeny of the UB organoid could be tracked by Venus expression. Co-staining of the engineered kidney structure with CDH1 and GATA3 specific antibodies identified a clear fusion of CDH1⁺/Venus⁻ distal nephron with CDH1⁺/Venus⁺ CD. Importantly, GATA3 expression was strong in the entire Venus⁺ CD structure, but progressively dropped along the distal-to-proximal axis of the distal nephron, as observed in vivo (FIG. 3D). Thus, the engineered kidney established a luminal interconnection between the nephron and CD, an essential morphological event for kidney function. Engineered kidney development was robust: approximately 80% of engineered kidneys underwent a similar developmental program, with most failures likely reflecting technical issues in manual construction (Table 9). Taken together, engineered kidney with interconnected nephron and CD can be efficiently generated from expandable NPCs and UBs.

TABLE 9
Summary of engineered kidney generation experiments.

| Genetic background of the | Wnt11- | Hoxb7- | Rosa26- |
|---|---|---|---|
| UB Organoids | RFP | Venus | Cas9/GFP |
| # of Engineered Kidney Re- | 50 | 34 | 8 |
| constructed | | | |
| # of Successful Engineered | 38 | 28 | 6 |
| Kidney Generated | | | |
| Successful Rate | 76% | 82.6% | 75% |

### Performing Efficient Gene Editing in the Expandable UB Organoid

The UB and CD models could provide an accessible in vitro complement to the mouse models for in-depth mechanistic studies and drug screening. Here, efficient gene overexpression (OE) or gene knockout (KO) would significantly extend the capability and utility of the in vitro model (FIG. 3E). As a proof of concept, GFP OE and GFP KO UB organoids were generated. For GFP OE, we used a standard lentiviral system to introduce GFP under the control of a CMV promoter. However, even at a very high titer, the lentiviral infection efficiency of the intact T-shaped UB was low. However, dissociating T-shaped UB or UB organoids into a single cell suspension prior to infection dramatically improved the infection efficiency. Widespread GFP activity was observed in resulting UB organoids after re-aggregation of infected cells (FIG. 3F). To test gene knockout, we targeted GFP in Rosa26-Cas9/GFP UB organoid, in which Cas9 and GFP are constitutively expressed from the Rosa26 loci. A mix of three different lentiviral constructs, expressing three different single guide RNAs (sgRNAs) with Cas9 targeting sites 100-150 bp apart, gave a highly-efficient, GFP sgRNA-specific, multiplexed CRISPR/Cas9 gene knockout, demonstrating effective gene knockout in UB organoid cultures (FIG. 3G).

Generating Human UB Organoids from Primary Human UPCs

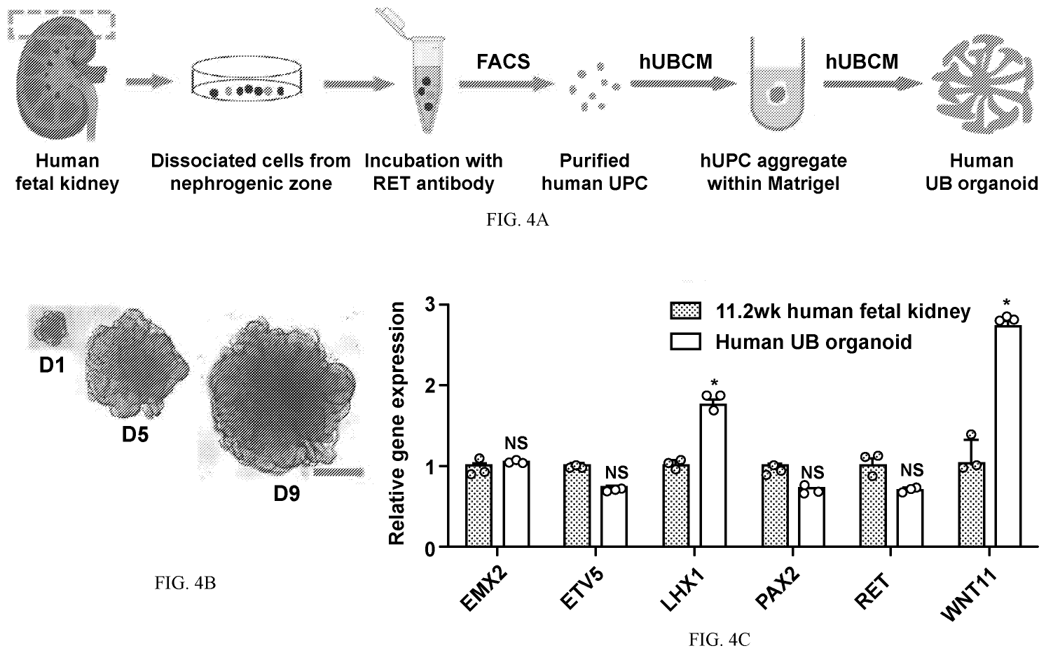

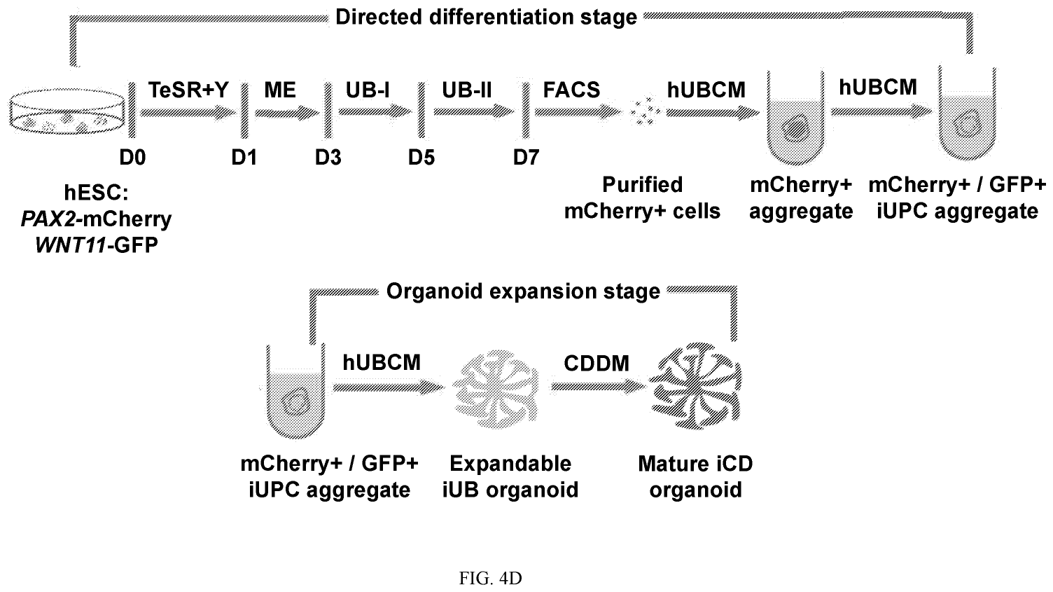

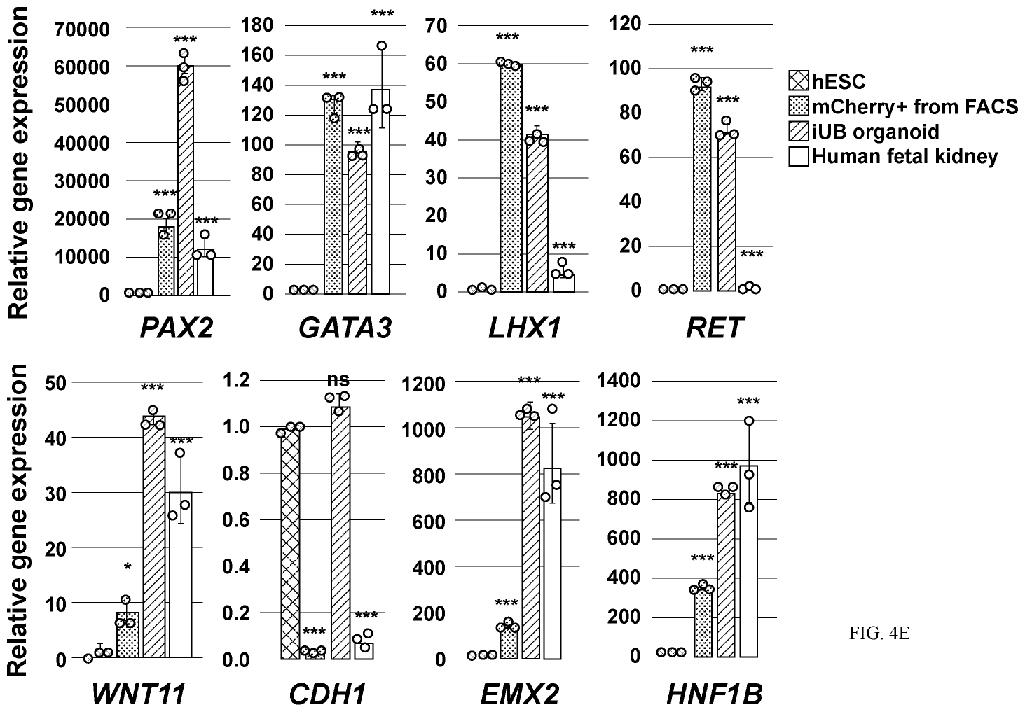

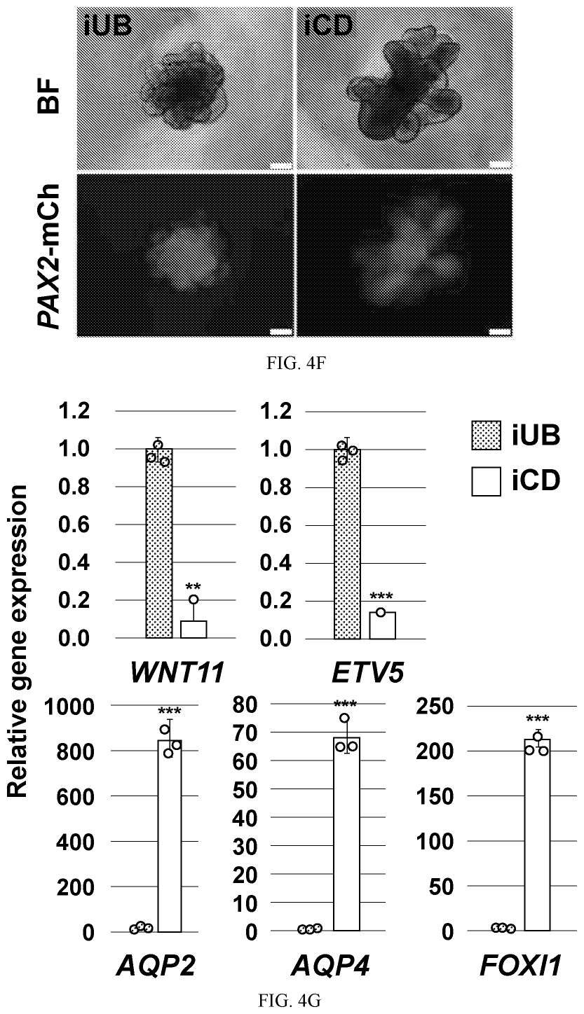

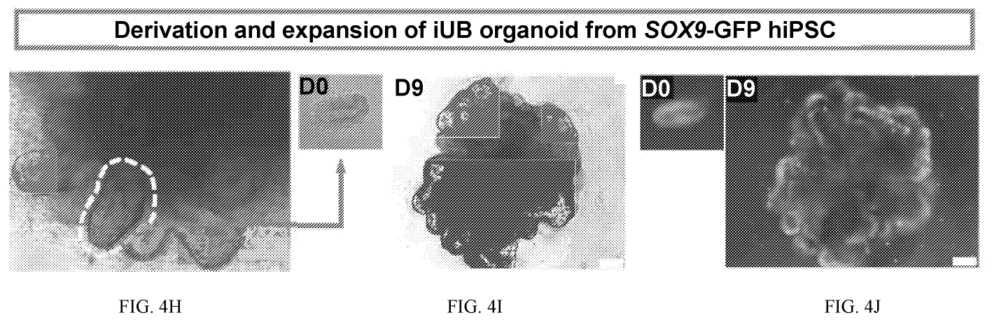

The successful generation of mouse UB and CD organoids prompted us to test whether the system can also derive human UB and CD organoids. To achieve this, we first developed a method to generate expandable human UB organoids from primary human UPCs (hUPCs) (FIG. 4A). Similar to their murine counterparts, hUPCs within UB tips, express RET and WNT11 (GUDMAP/RBK Resources, www.gudmap.org). Using an anti-RET antibody raised against the extracellular domain of RET that recognizes RET hUPCs, we performed FACS enrichment of RET cells from human kidneys between 9-13 weeks of gestational age and examined their growth in modified UBCM conditions. A robust human UBCM (hUBCM, Table 10) culture condition was identified that sustained the long-term expansion (an estimated 10⁸-10⁹ fold expansion over 70 days) as branching UB organoids (FIG. 4B). UPC marker gene expression was maintained in human UB cultures at level comparable to that of the human fetal kidney (FIG. 4C). Thus, we provide the first proof-of-concept that expandable human UB organoid can be derived from purified REV primary human UB progenitor cells.

TABLE 10
Medium recipe of Human UB culture medium version
1 (hUBCM-v1), listing supplements to a basal medium:
DMEM/F12 (1:1) (1×), Invitrogen #11330-032.

| | | | Final | |
|---|---|---|---|---|
| | | | Concen- | |
| Reagent Name | Company | Cat. No. | tration | |
| GlutaMAX-I (100×) | Invitrogen | 35050-079 | 1× | |
| MEM NEAA (100×) | Invitrogen | 11140-050 | 1× | |
| 2-Mercaptoethanol | Invitrogen | 21985-023 | 0.1 | mM |
| Pen Strep (100×) | Invitrogen | 15140-122 | 1× | |
| B-27, minus vitamin A | Invitrogen | 12587-010 | 1× | |
| ITS (100×) | Sigma | I3146-5ML | 1× | |
| LDN-193189 | Reagents Direct | 36-F52 | 200 | nM |
| TTNPB | TOCRIS | 0761 | 0.1 | μM |
| CHIR99021 | Reagents Direct | 27-H76 | 3 | μM |
| JAK inhibitor I | Stemcell | 74022 | 100 | nM |
| | Technologies | | | |
| GDNF (human) | PeproTech | 450-10-50 μg | 50 | ng/ml |
| A83-01 | STEMGENT | 04-0014 | 0.2 | μM |
| Rspo1 | R&D Systems | 4645-RS-100 | 100 | ng/ml |
| FGF7 | PeproTech | 100-19 | 50 | ng/ml |
| SB202190 | Axon Medchem | Axon 1304 | 5 | μM |
| Y27632 | Cayman | 10005583 | 10 | μM |
| | Chemical | | | |
| EGF | R&D Systems | 236-EG-200 | 50 | ng/ml |

TABLE 11
Medium recipe of Human UB culture medium version
2 (hUBCM-v2), listing supplements to a basal medium:
DMEM/F12 (1:1) (1X), Invitrogen #11330-032.

| | | | Final | |
|---|---|---|---|---|
| | | | Concen- | |
| Reagent Name | Company | Cat. No. | tration | |
| GlutaMAX-I (100×) | Invitrogen | 35050-079 | 1× | |
| MEM NEAA (100×) | Invitrogen | 11140-050 | 1× | |
| 2-Mercaptoethanol | Invitrogen | 21985-023 | 0.1 mM | |
| Pen Strep (100×) | Invitrogen | 15140-122 | 1× | |
| B-27, minus vitamin A | Invitrogen | 12587-010 | 1× | |
| ITS (100×) | Sigma | I3146-5ML | 1× | |
| LDN-193189 | Reagents | 36-F52 | 200 nM | |
| | Direct | | | |
| TTNPB | TOCRIS | 0761 | 0.1 μM | |
| CHIR99021 | Reagents | 27-H76 | For | 1 μM |
| | Direct | | iPSC- | |
| | | | derived | |
| | | | IUB | |
| | | | All | 3 μM |
| | | | other | |
| | | | hUB/ | |
| | | | iUB | |

| | | | |
|---|---|---|---|
| JAK inhibitor I | Stemcell | 74022 | 100 nM |
| | Technologies | | |
| GDNF (human) | PeproTech | 450-10-50 μg | 50 ng/ml |
| A83-01 | STEMGENT | 04-0014 | 0.2 μM |
| Rspo1 | R&D Systems | 4645-RS-100 | 100 ng/ml |
| FGF7 | PeproTech | 100-19 | 50 ng/ml |
| SB202190 | Axon | Axon 1304 | 5 μM |
| | Medchem | | |
| EGF | R&D Systems | 236-EG-200 | 50 ng/ml |

Generating iUB and iCD Organoids from Human Pluripotent Stem Cells

To determine whether UB and CD organoids could be generated from human pluripotent stem cell (hPSC)-derived UPCs, we first genetically engineered H1 human embryonic stem cells (hESCs) with a knockin dual reporter system where mCherry was expressed from the PAX2 locus (PAX2-mCherry) and GFP from the WNT11 locus (WNT11-GFP) (see Materials and Techniques). Using this reporter line, we first tested whether PAX2⁺/WNT11⁺ hUPCs can be generated following previously reported directed differentiation protocols that generated UB-like cells. After directed differentiation, we confirmed the expression of PAX2-mCherry, but failed to observe the expression of WNT11-GFP, indicating that the differentiation efficiency to generate hUPCs was relatively low following existing protocols. Relying on hUBCM's role in de novo hUPC induction and stabilization and a modified differentiation protocol, we were able to establish a stepwise protocol that resulted in high-quality hUPC cultures, which generated branching UB (iUB) organoids that underwent maturation to induced CD (iCD) organoids (FIG. 4D).

The UB is derived from the nephric duct (ND), which originates from primitive streak (mesendoderm)-derived anterior intermediate mesoderm. Consistent with this developmental trajectory, following a 7-day directed differentiation, we were able to first observe the expression of mesendoderm (ME) marker T on day 3 of differentiation in most cells, followed by the formation of large numbers of compact cell colonies that are GATA3⁺/SOX9⁺/PAX2⁺/PAX8⁺/KIT⁺/KRT8⁺ on day 7 of differentiation, indicating the generation of potential precursor cells of the UB lineage. Consistent with the immunostaining results, we were able to identify a PAX2-mCherry⁺ population (13.1%) by FACS on day 7. However, at this stage, the PAX2-mCherry⁺/WNT11-GFP⁺ population was very rare (0.4%), preventing further characterization or culture. However, further culture of PAX2-mCherry+ cells in the 3D/hUBCM culture conditions activated WNT11-GFP reporter expression at around 3 weeks, and the structure started to show a branching morphology (comparing between D17 and D25). We refer to the PAX2-mCherry⁺/WNT11-GFP⁺ branching structure an “iUB” organoid hereafter. Importantly, these iUB organoids could be expanded stably in 3D/hUBCM for at least 2 months without losing reporter gene expression (Table 12). Consistently, qRT-PCR analysis confirmed that WNT11 expression was low in the mCherry⁺ cells purified from FACS, but was dramatically elevated in the iUB organoid. Furthermore, even though UB marker genes PAX2, GATA3, LHX1 and RET were greatly elevated on day 7 of differentiation, while WNT11, CDH1, EMX2, and HNF1B, showed comparable levels of expression to the human fetal kidney only after extended hUBCM culture, indicating hUBCM promoted transition from a common nephric duct to a specific ureteric epithelial precursor (FIG. 4E). In addition to qRT-PCR, expression of marker genes SOX9 and CDH1 in the iUB organoid was detected at the protein level by immunostaining, further confirming the identity of the iUB organoid.

TABLE 12
Summary of human UB organoid derivation from different
sources and their expansion in vitro.

| Origins of human | Fetal | | |
|---|---|---|---|
| UB organoid | Kidney | hESC | hiPSC |
| Fetal tissue Age (week) | 9.1-12.3 | N/A | N/A |
| Knockin Reporters | N/A | PAX2-mCherry; | SOX9-GFP |
| | | WNT11-GFP | |
| Isolation Method | FACS | FACS | FACS |
| | (RET+) | (PAX2+) | (SOX9+) |
| Passage Method | Manual | Manual | Manual |
| Typical Passage Cycle (days) | 10 | 10 | 10 |
| Passage # | 7 | 6 | 3 |
| In Vitro Cultured | 70 | 60 | 30 |
| Time*** (Days) | | | |
| Reporter Expression (Days) | N/A | >60 | >30 |
| Fold of UB expansion | 10⁸~10⁹ | 10⁷~10⁸ | 10⁴~10⁵ |

***this is the culture time we achieved before our lab shutdown due to corona-virus outbreak. The maximum organoid culture time and expansion could be much greater. All data are presented as mean ± s.d.

To determine whether the expandable iUB organoid retained the potential to generate an iCD organoid after long-term expansion. iUB organoids were subjected to differentiation with the CDDM medium identified for mouse UB-to-CD transition. After 14 days of differentiation in CDDM, the human iUB organoid grew and elongated, maintaining PAX2-mCherry expression, but losing WNT11-GFP expression (FIG. 4F). More importantly, the expression of UPC markers WNT11 and RET was greatly diminished, while CD marker genes AQP2, AQP4 and FOXI1 were dramatically elevated, indicating the successful transition from iUB to iCD (FIG. 4G).

To test whether expandable iUB organoids could be generated from human induced pluripotent stem cells (hiPSCs), we employed SOX9-GFP hiPSC for differentiation and purified the SOX9-GFP⁺ UB precursor cells on day 7 of differentiation (via flow cytometry analysis of GFP+ cells differentiated from SOX9-GFP reporter hiPSCs, showing 32.7%). Similar to hESC-derived iUBs, following an extended culture in hUBCM, we were able to derive SOX9-GFP iUB organoids that expanded stably with retained SOX9-GFP expression throughout (Table 12, and FIG. 4H-4J). Taken together, these results support the conclusion that expandable iUBs and mature iCDs organoid can be derived from hESC and hiPSC lines.

Generating iUB and iCD Organoids Independent of Reporter hPSC Lines

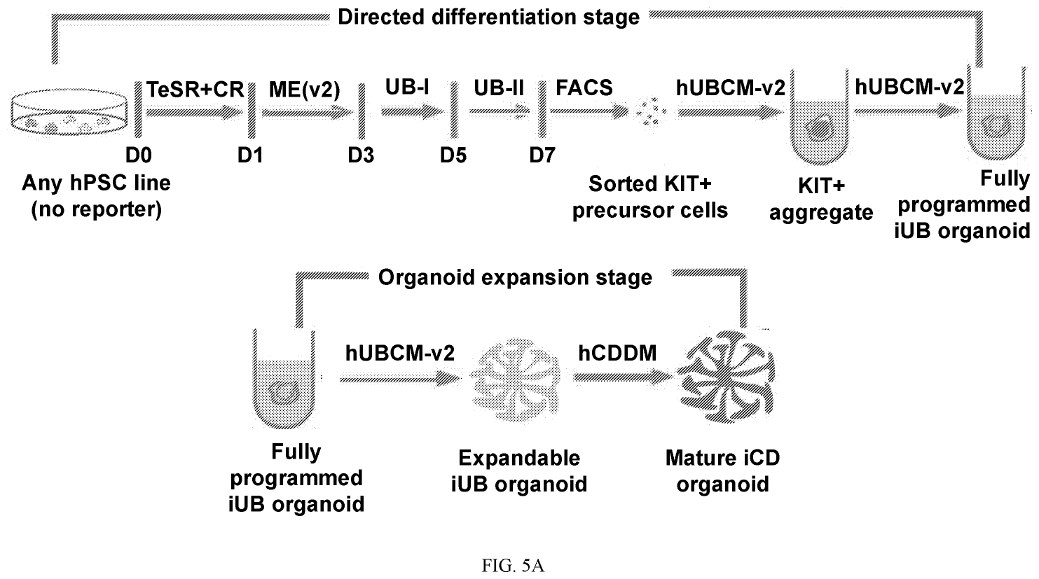

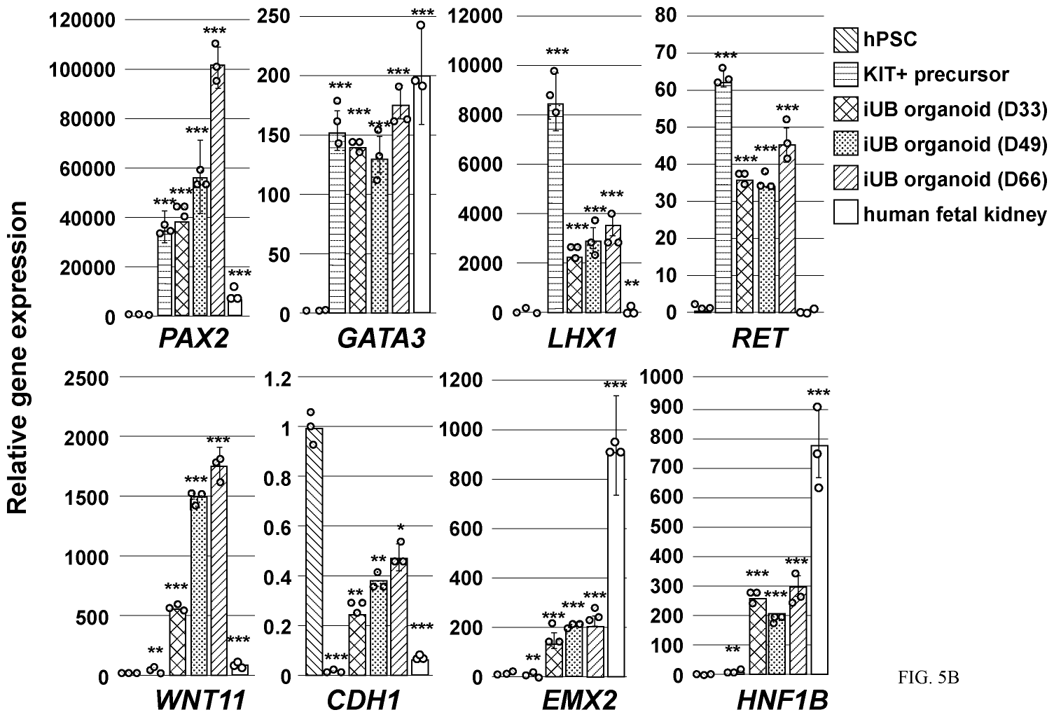

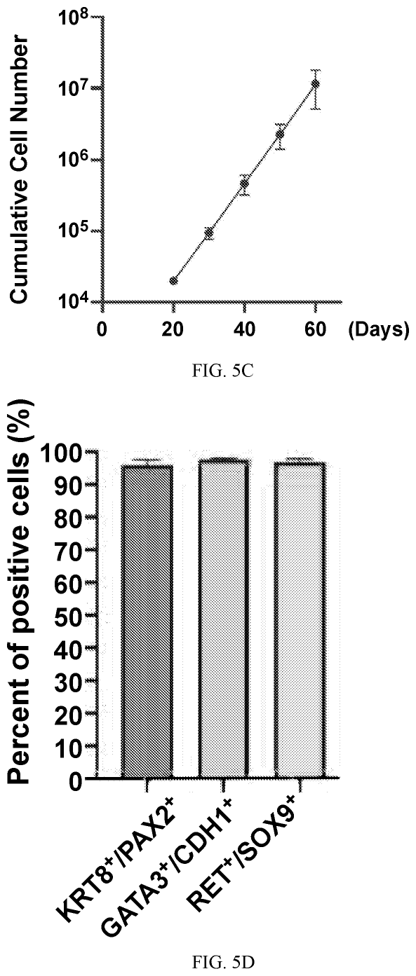

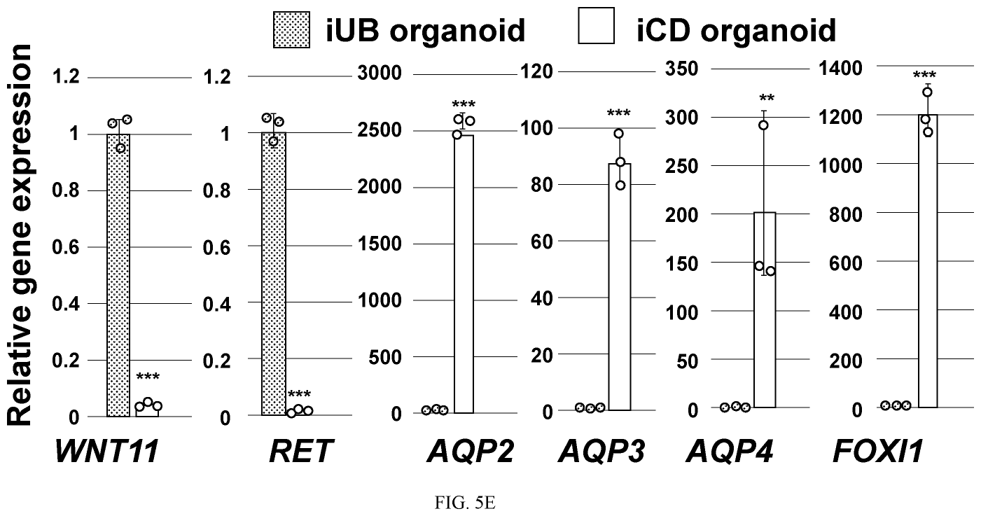

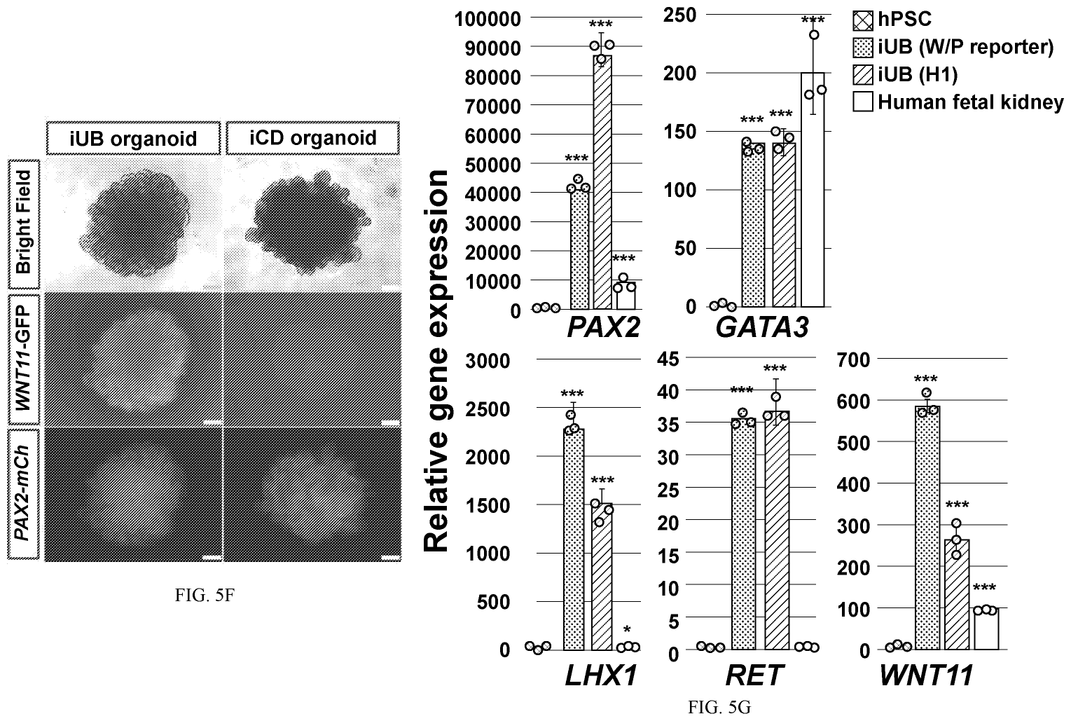

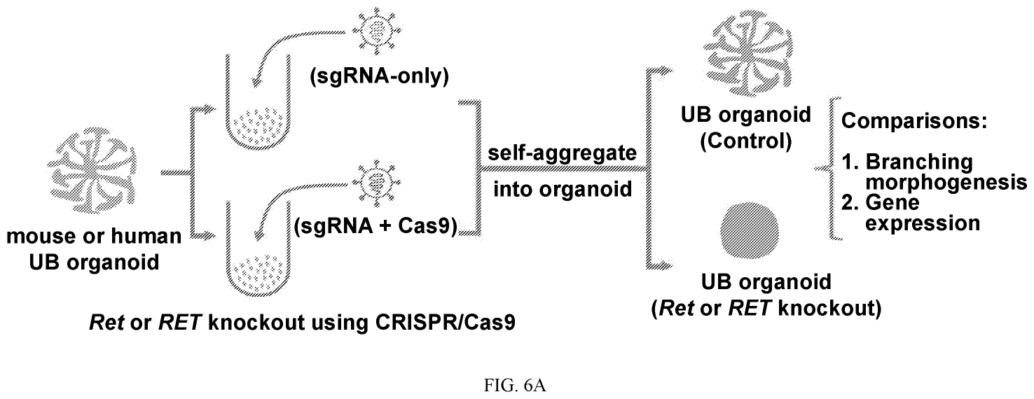

The reporter hPSC lines are useful in developing iUB differentiation protocols, but if iUB organoids can only be derived from these reporter hPSCs, its applications will be significantly limited. To solve this problem, we next developed a method to derive iUB organoid from any given hPSC line in the absence of reporter (FIG. 5A). In this method, after 7 days of differentiation, sorting of KIT⁺ cells was used to enrich the precursor population, rather than sorting based on PAX2-mCherry or SOX9-GFP reporters. With further refinement of our stepwise iUB differentiation protocol and a refined hUBCM (hUBCM-v2, Table 11), long-term expandable branching iUB organoids can be derived within 12 days from hPSCs with high efficiency. Following this protocol, as proof-of-concept, we have successfully derived iUB organoids from three different hPSC lines, including two hESC lines and one hiPSC line.

KIT was previously reported as a surface marker that can be used to enrich UB-like cells upon hPSC differentiation. Interestingly, we noticed that KIT⁺ cells are frequently co-stained with PAX2. We thus hypothesized that FACS of KIT⁺ cells will enrich for PAX2⁺ precursor cells similar to the sorting of PAX2-mCherry⁺ cells using our PAX2-mCherry reporter line. Starting from our WNT11-GFP/PAX2-mCherry hESC line, after 7 days of differentiation, 36.1% of the cells were KIT⁺ (measured via flow cytometry analysis of KIT⁺ precursor cells differentiated from the dual reporter hESC line). Further culture of these KIT⁺ cells in hUBCM-v2 showed a much faster induction of WNT11-GFP expression than using hUBCM (5-7 days with hUBCM-v2 vs. ˜3 weeks with hUBCM). Importantly, accompanying the expression of WNT11-GFP, the organoid started to show the typical branching morphology, and can since be stably passaged and expanded billions of folds either manually or as single cells for at least 70 days (FIG. 5C, Table 13).

TABLE 13
Summary of human iUB organoids derivation from different
hPSC lines independent of reporter and their expansion
in vitro. Total culture time are up until manuscript submission.
The maximum organoid culture time and expansion could
be longer. All data are presented as mean ± s.d.

| Origins of Human | | | |
|---|---|---|---|
| UB Organoid | hESC | hESC | hiPSC |
| Cell lines | H1 (WA01) | PAX2-mCherry; | SOX9-GFP |
| | | WNT11-GFP | |
| Isolation Method | FACS (KIT+) | FACS (KIT+) | FACS (KIT+) |
| Passage Method | Manual or | Manual or | Manual or |
| | Single Cell | Single Cell | Single Cell |
| Freeze-Thaw | Yes | Yes | Yes |
| Typical Passage | 6-10 | 6-10 | 6-10 |
| Cycle (days) | | | |
| Passage # | 8 | 8 | 8 |
| In Vitro Cultured | 70 | 72 | 72 |
| Time until | | | |
| submission (Days) | | | |
| Reporter Expression | N/A | Yes | Yes |
| Fold of UPC | 10⁸~10⁹ | 10⁸~10⁹ | 10⁷~10⁸ |
| expansion | | | |

To determine whether gene expression in the iUB organoid is also stably maintained over long-term culture, we collected iUB organoids 33 days, 49 days and 66 days after the initiation of culture in hUBCM-v2, and compared their gene expression by qRT-PCR with undifferentiated hPSCs, KIT⁺ precursor cells, and human fetal kidney tissue. Consistent with our previous finding with sorted PAX2-mCherry⁺ precursors (FIG. 4G), the sorted KIT⁺ precursor cells also showed strong expression of PAX2, GATA3, LHX1 and RET, but the expression of WNT11, CDH1, EMX2 and HNF1B were only induced after further programming in the presence of hUBCM-v2 (FIG. 5B). Importantly, gene expression of all these markers in the iUB organoids were maintained stably throughout the culture period, at levels comparable or higher than that of human fetal kidney tissue, indicating the robustness of our method. Wholemount immunostaining or section staining of the iUB organoids for various general UB lineage markers (KRT8, PAX2, PAX8, GATA3 and CDH1) or UB tip markers (RET and SOX9) further confirmed the expression of all these genes at the protein level. Importantly, quantification of the immunostaining results indicated that more than 95% of cells in the organoids showed homogeneous expression of all markers (FIG. 5D), indicating the majority of the cells in the iUB organoid are of the UB progenitor identity, which has never been achieved before.

To determine whether the iUB organoid can generate iCD organoid, we further developed a refined CDDM (hCDDM, Table 7) which efficiently induced the mRNA expression of various PC (AQP2, AQP3 and AQP4) and IC (FOXI1) markers hundreds to thousands of fold within 14 days of differentiation from iUB organoids, accompanying the reduction of UB tip genes WNT11 and RET (FIGS. 5E and 5F). Given the limited availability of validated antibodies, we were only able to examine AQP3 and FOXI1 at the protein level. Approximately, 20-30% of iCD organoid cells were AQP3⁺ but we were not able to observe FOXI1⁺ ICs. These results indicate long-term expandable iUB organoids generate PC and IC-like cells but cells generated in hCDDM do not attain a fully mature CD cell fates.

Importantly, similar iUB organoids were also generated from a second hESC line (H1) and from the SOX9-GFP reporter hiPSC line. After 7 days of differentiation, 55.4% of H1 cells and 43.9% of SOX9-GFP hiPSCs were identified as KIT⁺ on FACS. Further culturing of these KIT⁺ cells in hUBCM-v2 derived iUB that can be stably expanded as branching iUB organoids (from wild-type H1 hESC). qRT-PCR further confirmed that gene expression in the H1 hESC-derived iUB organoid is similar to the iUB organoid derived from our dual reporter hESC line shown above (FIG. 5G). Similarly, qRT-PCR (qRT-PCR analyses of the FACS purified KIT⁺ precursor and KIT⁺ iUB organoids cultured for 53 days for various markers: PAX2, GATA3, LHX1, RET, WNT11, CDH1, EMX2, HNF1B; Undifferentiated hiPSCs and human fetal kidney (11.2 week gestational age) were used as controls), whole-mount immunostaining, and section staining confirmed that the majority of the cells in the hiPSC-derived iUB organoids are of the UB progenitor identity.

### Modeling Kidney Development and Disease Using Mouse and Human UB Organoids

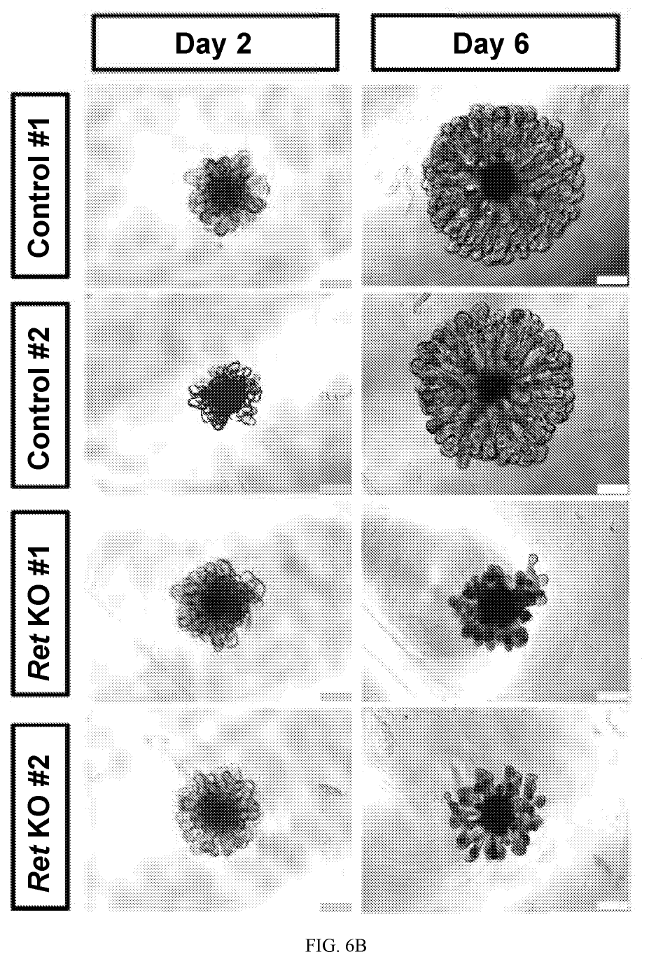

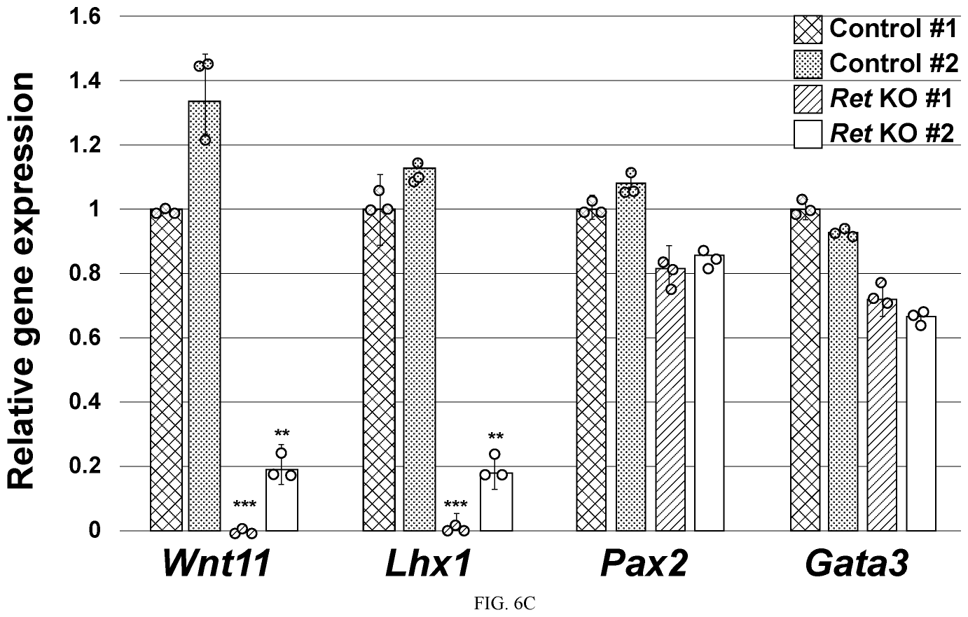

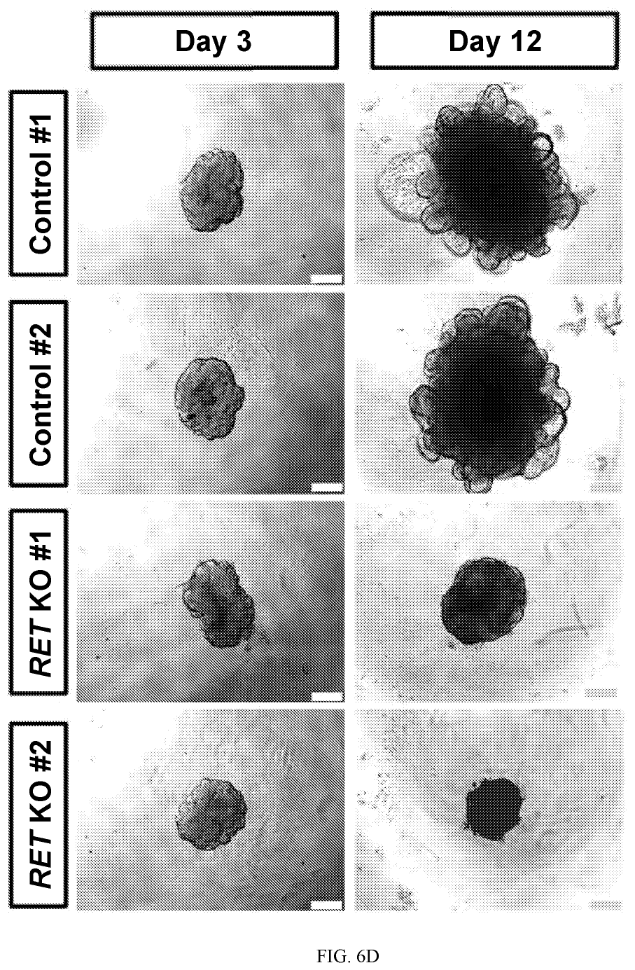

GDNF is a critical signal in both mouse and human UB culture. In vivo, GDNF secreted by metanephric mesenchyme cells surrounding UPC-containing branch tips signals via RET, with its co-receptor GFRA1, to maintain the UPC state and stimulate UB branching morphogenesis. Loss of the activity of these genes results in a CAKUT syndome. We employed CRISPR/Cas9 system to knock out Ret/RET in mouse and human UB organoids predicting as in vivo, UB organoid development in vitro would be Ret/RET-dependent (FIG. 6A). UB organoids were infected with lentivirus expressing Cas9 and two independent sgRNAs targeting Ret/RET (in lentiCRISPR-v2 vector), while a control group received sgRNA without Cas9 (in lentiGuide-puro vector). As expected, the control mouse UB organoids grew normally with maintained branching morphogenesis upon lentiviral infection and puromycin selection, while both Ret knockout (KO) UB organoids stopped branching (FIG. 6B). Whole-mount immunostaining of control and Ret KO UB organoids confirmed a dramatic reduction (more than 95%) of RET expression 6 days after lentiviral infection in the Ret KO UB organoids, demonstrating the successful removal of Ret. Consistent with the defect in branching morphogenesis in the Ret KO organoids, genes enriched in UB tip, Wnt11 and Lhx1, were reduced dramatically, and the expression of common UB lineage markers Pax2 and Gata3 were also decreased (FIG. 6C). Knockout of RET in the human iUB organoid also resulted in the arrest of branching morphogenesis in both of the RET KO organoids receiving two different sgRNAs (FIG. 6D) and a loss of RET immunostaining in more than 95% of cells with both sgRNAs. Interestingly, even though WNT11 and GFRA1 expression was significantly reduced in both RET KO iUB organoids, the expression of LHX, GATA3, PAX2, as well as ETV5 and SOX9, did not show consistent changes in both RET KO organoids. These results indicate that species-specific regulatory network downstream of Ret/RET might govern UB progenitor fate, consistent with previous observations of convergent and divergent mechanisms of nephrogenesis between mouse and human. Taken together, we provide a proof-of-concept for recapitulating kidney development and disease using mouse and human UB organoid models.

Overall in this study, we report 3D culture models enabling the expansion and differentiation of mouse and human UB progenitor cells. The organoid culture medium effectively replaces cell interactions within the nephrogenic niche of the developing mammalian kidney, with a chemically-defined synthetic niche capable of maintaining UB progenitor cell identity. Consistent with mouse genetics studies, signaling pathways that play key roles in kidney branching morphogenesis, such as GDNF, FGF, RA and Wnt signaling, are also essential in maintaining UB progenitor cell identity in UB organoids. UPC cloning efficiency is not very high in the UBCM culture. Similar to our 3D NPC culture, it is likely that cell-cell contact is important for maintaining the best tip identity, as aggregated UPCs, or manually passaged UB organoids as small cell clusters, can maintain Wnt11-RFP homogeneously. Better understanding of cell-cell contact and/or potential additional paracrine signals might help further improve the culture, thus allowing the development of a more robust clonal expansion method.

Leveraging our ability to produce large quantities of high-quality UB progenitor cells in the format of expandable branching UB organoids, we performed a screening that identified CDDM a cocktail of growth factors, small molecules and hormones that together can differentiate UB organoids into CD organoids with spatially patterned mature PCs and ICs. The molecular mechanisms underlying the UB-to-CD transition are still largely unknown. The in vitro organoid system provides a new tool to study this process, and the chemically-defined components in CDDM shed new light on potential signals that trigger CD maturation in vivo. Despite the general difficulty of maturing stem cell-derived tissues, our study shows that it is possible to achieve proper patterning and maturation in vitro, similar to what we observe in vivo, when starting from high-quality progenitor cells under appropriate culture conditions. The limited number of available antibodies precludes a more comprehensive characterization of the mature human PC and IC state. The current platform is a strong base for future studies to further improve CD cell maturation and assess physiological activity of differentiated cell types.

An interesting observation during the de novo human UB directed differentiation process was the induction of hUPC fate by hUBCM or hUBCM-v2. One possibility to explain this phenomenon is that these media could stabilize the rare and transient hUPC population generated from directed differentiation. Another possibility is that these media could promote cell fate transition from earlier WD-staged precursor cells to the hUPC fate. In support of both possibilities, our NPC culture medium, NPSR, has recently been reported to facilitate the generation of NPC-like cells in both directed differentiation and transdifferentiation settings. Future studies are warranted to understand how hUBCM and hUBCM-v2 contributes to hUPC fate specification.

The generation of an engineered kidney from expandable NPCs and UPCs provides a proof-of-concept for rebuilding a kidney in vitro from kidney-specific progenitor cells. The availability of expandable NPCs and UPCs provides the scalable building blocks required for making a kidney. The interaction between NPCs and UPCs is faithfully recapitulated, leading to the autonomous differentiation into interconnected nephron and collecting duct structures. Interestingly, different from prior study, in our engineered kidney system, interstitial progenitor cells appear to be dispensable in reconstructing a branching kidney structure in vitro. It is likely that one of our engineered kidney culture medium components, TTNPB (alternative names: AGN 191183, Arotinoid Acid, or Ro 13-7410), a small molecule analog of RA, substitutes RA production by interstitial progenitor cells, an essential mechanism for proper UB branching and kidney development in vivo. Future efforts will require the integration of vascular progenitor cells, and a more in-depth evaluation of interstitial progenitor cells, to develop engineered structures for testing in animal models of organ transplantation.

Efficient genome editing in UB organoids opens up many new applications using the UB and CD organoid platform. UB and CD organoids can be generated from available transgenic mouse strains that bear genetic mutations related to kidney development and disease. In addition, disease-relevant mutations can be introduced into the UB organoid directly, enabling the investigation of pathophysiology throughout the entire course of kidney branching morphogenesis, from the UB branching period to the mature CD stage. Our proof-of-concept Ret/RET knockout experiment demonstrated that our UB organoid system can recapitulate genetic malfunction of branching morphogenesis in vitro as that of in vivo. Our results also shed light on the potential different regulatory mechanisms downstream of Ret/RET in mouse and human. Our system offers a unique platform to further investigate how human RET mutations identified in the human CAKUT patients might contribute to congenital kidney malformation. The ability to produce large quantities of UB and CD organoids also provides a platform for drug screening. The mature PCs and ICs present in the CD organoids are potential sources for cell replacement therapies for patients with CD damage. In conclusion, the new UB and CD organoid system provides a powerful tool for studying kidney development, modeling kidney disease, discovering new drugs and, ultimately, regenerating the kidney.

### Materials and Techniques

### Human Tissues

All human fetal kidney samples were collected under Institutional Review Board approval (USC-HS-13-0399 and CHLA-14-2211). Following the patient decision for pregnancy termination, the patient was offered the option of donation of the products of conception for research purposes, and those that agreed signed an informed consent. This did not alter the choice of termination procedure, and the products of conception from those that declined participation were disposed of in a standard fashion. The only information collected was gestational age and whether there were any known genetic or structural abnormalities.

### Mice

All animal work was performed under Institutional Animal Care and Use Committee approval (USC IACUC Protocol #20829). Swiss Webster mice were purchased from Taconic Biosciences (Model #SW-F, MPF 4 weeks). Sox9-GFP mice were kindly shared from Dr. Haruhiko Akiyama³⁷. Wnt11-RFP mice (JAX #018683), Hoxb7-Venus mice (JAX #016252) and Rosa26-Cas9/GFP (JAX #026179) were obtained from the Jackson Laboratory.

hPSC Lines

Experiments using hPSCs were approved by the Stem Cell Oversight Committee (SCRO) of University of Southern California under protocol #2018-2. Human pluripotent stem cells are routinely cultured in mTeSR1 medium in monolayer culture format coated with Matrigel and passaged using dispase.

### 3D Cultured NPC Lines

The 3D cultured NPC lines we used in this study were derived from E11.5 whole kidney cells of the wild-type Swiss Webster mouse strain, using an improved method we developed that can derive NPC lines from any mouse strain without the need for prior purification of NPCs. These NPCs had been cultured 6-12 months (billions of billion-fold of expansion) before used for reconstruction of engineered kidney with UB organoids.

### Screening for Optimal UB Culture Condition

We systematically screened the optimal UB culture condition in four different stages (Stages I-IV). In Stage I, we tested the most updated UB culture condition from literature, Yuri et al., 2017, in which FGF1, retinoic acid (RA), CHIR99021, and GDNF were used to allow the growth of isolated UBs in vitro. By repeating this condition, we confirmed that this medium supported very well the growth and branching of the UB in the first 4-5 days. However, after that, UB growth slowed down significantly, and more importantly, Wnt11-RFP expression was dramatically decreased, indicating that further optimization was needed to selectively expand the Wnt11+ UB progenitor cells.

In Stage II, we optimized the individual medium components employed by Yuri et al. (FGF1, RA, CHIR99021, and GDNF). We first asked if each individual factor was necessary in the medium. For this, the factors were withdrawn from the medium individually, and the results clearly showed that all these factors were essential for maintaining the branching of UB. Then we further optimized these factors. RA is known to be unstable in tissue culture, so we replaced it with another widely used small molecule RA substitute TTNPB. CHIR99021 was used at 1p M by Yuri et al., but based on our own experiences and literature, different doses of CHIR99021 often have different biological effects. So we titrated it from 1p M, 3p M, to 6 μM, from which we identified 3 μM to be the optimal concentration. To optimize for FGF1, we tested different members from the FGF family, including FGF2, FGF4, FGF7, FGF8, FGF9, FGF10, and FGF20, from which we identified FGF9 to be superior in supporting Wnt11-RFP expression than FGF1. GDNF was unchanged in the medium, considering its essential role in maintaining the UB progenitor population in vitro and in vivo.

In Stage III, based on the optimized recipe consisting of FGF9, TTNPB, CHIR99021 (3 μM) and GDNF, we preformed our 1^{st} round of screening of growth factors and small molecules targeting major developmental pathways (e.g. TGF-β, BMP, Wnt, FGF, Hedgehog and Notch) and others. The branching morphogenesis, growth rate and Wnt11-RFP were recorded as readouts (Table 1). From this, we identified several hits that improved either the UB growth rate or Wnt11-RFP, or both. Representative images were taken at least for LDN193189, A83-01, and R-Spondin 1. These individual hits were then subjected to a 2^{nd} round of screening to test their effects in various combinations, eventually leading to the identification of the optimal UB progenitor culture medium UBCM consisting of FGF9, TTNPB, CHIR99021 (3 μM), GDNF, LDN193189, A83-01, R-Spondin1, JAKI and SB202190. Representative images were taken at least for the combinatorial effect of JAKI and SB2020190, for which only marginal effects were observed when used individually.

Lastly, in Stage IV, we asked whether each of the components in UBCM was essential. The factors were removed from the UBCM individually and the results indicated that all of them were necessary to achieve optimal UB organoid branching and to sustain Wnt11-RFP expression.

### Derivation of Mouse UB Organoid

### From Intact T-Shaped UB:

Male mice with the desired genotype (Wnt11-RFP, Hoxb7-Venus, Sox9-GFP, or Rosa26-Cas9/GFP) were mated with female Swiss Webster mice. Plugs were checked the next morning; midday of plug positive was designated as embryonic day 0.5 (E0.5). Timed pregnant mice were euthanized at E11.5. Kidneys were dissected out from embryos using standard dissection techniques and transferred into a 1.5 mL Eppendorf tube on ice. Next, at least 500 μL fresh, pre-warmed collagenase IV (Thermo Fisher, Cat. No. 17104019) was added into the tube and incubated at 37° C. for 20 minutes. After incubation, collagenase was removed and at least 500 μL of 10% FBS (1×DMEM, 1× GlutaMAX-I, 1×MEM NEAA, 0.1 μM 2-Mercaptoethanol, 1× Pen Strep, 10% FBS) was added to resuspend the kidneys. 1-3 kidneys were transferred each time with 80-100 μL medium onto a 100 mm petri dish lid as a working droplet. UBs were isolated from the surrounding MM and other tissues using sterile needles (BD, Cat. No. BD305106) without damaging UBs. The isolated UBs can be temporarily left in the medium at room temperature for <30 minutes while dissecting other UBs. After all UBs were isolated, each UB was transferred together with 1-3 μL medium into an 8 μL cold Matrigel droplet at the bottom of one well of a U-bottom 96-well low-attachment plate, by using a P10 micropipette. The UB and Matrigel were mixed by pipetting gently 2-3 times. After all UBs were embedded in Matrigel, the plate was incubated at 37° C. for 20 minutes for the Matrigel to solidify. Then, 100 μL of mouse UBCM (mUBCM) was slowly added into each well and the plate was then transferred into an incubator set at 37° C. with 5% CO₂.

From dissociated UB single cells:

For deriving UB organoid from dissociated UB single cells (e.g. for gene editing purpose), after the isolation of E11.5 T-shaped UBs from kidneys following the procedures described above, all UBs were collected into a 1.5 mL Eppendorf tube with the medium removed as much as possible. An appropriate amount (e.g. for 20 UBs, we use 200 μl, adjust accordingly) of pre-warmed Accumax cell dissociation solution (Innovative Cell Technologies, #AM105) was added into the tube, and the tube was then incubated at 37° C. for 20 minutes and gently tapped every 7-10 minutes. Then, an equal amount of 10% FBS was added into the tube to neutralize the Accumax and the mixture was pipetted gently 8-10 times to dissociate the UB into single cells. The tube was then centrifuged at 300×g for 5 minutes. After centrifugation, the supernatant was carefully removed and UB cells were resuspended in an appropriate amount of mUBCM (Y27632 was supplemented at 10 μM final concentration for the first 24 hours) by pipetting gently 6-8 times. Cell density was measured using automatic cell counter (Bio-Rad, TC20). ˜2,000 cells were transferred into each well of a U-bottom 96-well lowattachment plate and extra amount of mUBCM (with 10 μM Y27632) was added to the well to make the final volume 100 μl per well. The plate was then centrifuged at 300×g for 3 minutes and transferred and cultured in a 37° C. incubator. After 24 hours, the ˜2,000 single cells formed an aggregate autonomously and the aggregate was then transferred together with 1-3 μl medium into an 8 μl cold Matrigel droplet in another well of the U-bottom 96-well low-attachment plate using a P10 micropipette. The aggregate was pipetted gently 2-3 times to mix with Matrigel. After all aggregates were embedded in Matrigel, the plate was incubated at 37° C. for 20 minutes for the Matrigel to solidify. Lastly, 100 μl of UBCM was added slowly into each well and the plate was then transferred into an incubator set at 37° C. with 5% CO₂.

### Mouse UB Organoid Expansion and Passaging

Mouse UBCM was renewed with fresh medium every two days, and UB organoid was passaged every five days.

Manual Passaging as Small Tips:

UB organoid (with Matrigel) was first transferred from U-bottom 96-well plate onto a 100 mm petri dish lid with 80-100 μL medium using a P1000 pipette with the tip cut 0.5-1 cm to widen the diameter. Most of the Matrigel surrounding the organoid was removed using sterile needles under a dissecting microscope. A small piece of the organoid with 3-5 branching tips was cut using needles and then re-embedded into Matrigel droplet in a U-bottom 96-well low-attachment plate well and cultured in a 37° C. incubator following the same embedding procedure described above.

Passaging as Single Cells:

UB organoid (with Matrigel) was first transferred from U-bottom 96-well plate onto a 100 mm petri dish lid with 80-100 μL medium using a P1000 pipette with the tip cut 0.5-1 cm to widen the diameter. Most of the Matrigel surrounding the organoid was removed using sterile needles under a dissecting microscope. Organoid was then cut into small pieces using sterile needles (the smaller the piece, the easier to dissociate). All the pieces were transferred into 1.7 mL Eppendorf tubes (1-3 organoids per tube) with as little medium as possible using a P200 pipette, extra medium was removed from the tube. 200-400 μL of pre-warmed Accumax cell dissociation solution was added into the tube. The tube was then incubated in 37° C. for 20 minutes and gently tapped a few times every 7-10 minutes. After the incubation, 200-400 μL of 10% FBS was added to neutralize the Accumax and the mixture was pipetted gently 6-8 times to further dissociate the organoid into single cells. The tube was then centrifuged at 300×g for 5 minutes. After centrifugation, supernatant was carefully removed and the UB cell pellet was resuspended in appropriate amount of mUBCM (with the addition of Y27632 at 10 μM final concentration) by pipetting up and down gently 6-8 times. Cell density was measured by automatic cell counter. ˜2,000 cells were transferred into each well of a U-bottom 96-well low-attachment plate and extra amount of mUBCM (with 10 μM Y27632 for the first 24 h) was added to the well to make the final volume 100 μL per well. The plate was then centrifuged at 300×g for 3 minutes and then transferred and cultured in a 37° C. incubator. After 24 h, the ˜2,000 single cells formed an aggregate autonomously and the aggregate was then embedded into Matrigel droplet in another well of the U-bottom 96-well low-attachment plate and cultured in a 37° C. incubator following the same embedding procedure described above.

### Derivation of Human UB Organoid

Organoid derivation from RET+ primary UPCs purified from human fetal kidney:

All human fetal kidney samples were collected under Institutional Review Board approval (USC-HS-13-0399 and CHLA-14-2211). Following the patient decision for pregnancy termination, the patient was offered the option of donation of the products of conception for research purposes, and those that agreed signed an informed consent. This did not alter the choice of termination procedure, and the products of conception from those that declined participation were disposed of in a standard fashion. The only information collected was gestational age and whether there were any known genetic or structural abnormalities. The kidney nephrogenic zone was dissected manually from each of fresh 9-13-week human fetal kidney, chopped into small pieces with surgical blade, and divided into 4-6 1.5 mL Eppendorf tubes. Tissues were washed with PBS and resuspended with 500 μL of pre-warmed Accumax per tube and the tubes were incubated at 37° C. with shaking for 25 minutes. 500 μL 10% FBS was then added to neutralize the Accumax, and the mixture was pipetted ˜25 times to dissociate the tissues into single cells. The mixture medium with kidney cells were then pooled together and sieved through a 40 μm cell strainer, then transferred into 1.5 mL Eppendorf tubes, centrifuged at 300×g for 5 minutes and the supernatant was carefully removed. All cell pellets from this preparation were then resuspended and combined into 300-400 μL cold FACS medium (1×PBS, 1× Pen Strep, 2% FBS) supplemented with a human anti-RET antibody at 1:200 dilution into one tube and incubated for 30 minutes on ice. The tube was gently tapped every 10 minutes to ensure mixing. After 30 minutes, 1 mL cold FACS medium was added into the tube. The tube was then centrifuged at 300×g for 5 minutes and the supernatant was carefully removed. Cells pellet was resuspended again in 500 μL cold FACS medium plus secondary antibody (Donkey anti-Goat, Alexa Fluor 568, Invitrogen, Cat. #A-11057) at 1:1000 dilution and incubated for 30 minutes on ice, with gentle mixing every 10 minutes. After the incubation, 1 mL cold FACS medium was added into the tube. The tube was then centrifuged at 300×g for 5 minutes and the supernatant was carefully removed. The pelleted cells were resuspended with 300-500 μL cold FACS medium plus DAPI at 1:2000 ratio, placed through 40 μm cell strainer and transferred into a FACS tube on ice before FACS. RET (Alexa Fluro 568) UPCs were then sorted out by FACS. The RET+ cells were collected in a 1.5 mL Eppendorf tube with 500 μL 10% FBS. The tube was centrifuged at 300×g for 5 minutes and the supernatant was carefully removed. Cell pellet was then resuspended in an appropriate amount of hUBCM (with the addition of Y27632 at 10 μM final concentration) and cell density was measured by automatic cell counter. ˜2,000-20,000 cells were transferred into each well of a U-bottom 96-well low-attachment plate and an appropriate amount of hUBCM (with 10 μM Y27632 for the first 24 h) was added to the well to make the final volume of 100 μL per well. After 24 h, UB cell aggregate was formed and embedded into an 8 μL cold Matrigel droplet in another well of the U-bottom 96-well low-attachment plate and cultured in a 37° C. incubator following the same embedding procedure described above (with hUBCM). After approximately 10-15 days of culture, epithelial tip structures could be seen budding out from the aggregate. These tip structures were dissected out and re-embedded into Matrigel and expanded as human UB organoid.

Organoid Derivation from Human ESCs and iPSCs

Human pluripotent stem cells are routinely cultured in mTeSR1 (TeSR) medium in monolayer culture format coated with Matrigel and passaged using dispase. The hPSCs were pre-treated with 10 μM Y27632 in TeSR medium for 1 h before dissociation into single cells using Accumax cell dissociation solution. Following dissociation, ˜60,000 cells were seeded into Matrigel coated 12-well plate with 1 mL TeSR medium with 10 μM Y27632 (first protocol, FIG. 4D) or 1× CloneR (STEMCELL Technologies, Cat. No. 05888) (second protocol, FIG. 5A) (day 0). 24 h later (day 1), the medium was removed and 1 mL of pre-warmed ME stage medium was slowly added to the well. 48 h later (day 3), ME stage medium was removed and 1 mL of UB-I stage medium was slowly added to the well. 24 h later (day 4), medium was changed to 1 mL of fresh UB-I medium again. After another 24 h (day 5), UB-I medium was removed, and 1.5-2 mL of UB-II stage medium was slowly added to the well. 24 h later (day 6), medium was changed to 1.5-2 mL of fresh UB-II medium. At day 7, differentiated cells were dissociated into single cells following the standard Accumax dissociation method. For wild-type hESC line without any reporter, cell pellet after dissociation was resuspended in 250-400 μL cold FACS medium (1×PBS, 1× Pen Strep, 2% FBS) supplemented with a PE conjugated anti-human CD117(C-KIT) antibody (Biolegend, Cat. No. 313204) at 1:200 dilution and incubated for 30 minutes on ice. The tube was gently tapped every 10 minutes to ensure mixing. After 30 minutes, 1 mL cold FACS medium was added into the tube. The tube was then centrifuged at 300×g for 5 minutes and the supernatant was carefully removed. The pelleted cells were resuspended with 300-500 μL cold FACS medium plus DAPI at 1:2000 ratio, placed through 40 μm cell strainer (Greiner bio-one, Cat. No. 542040) and transferred into a FACS tube on ice before FACS. C-KIT⁺ (PE) cells were then sorted out by FACS. For reporter cell lines (PAX2-mCherry/WNT11-GFP hESC line or SOX9-GFP hiPSC line), dissociated cells were either resuspended directly in 300-500 μL cold FACS medium plus DAPI at 1:2000 ratio, or first went through the C-KIT staining process described above before resuspended in FACS medium with DAPI. These cells were then put through a 40 μm cell strainer and placed on ice before FACS. mCherry⁺ cells (from the PAX2-mCherry/WNT11-GFP hESC line), GFP⁺ cells (from the SOX9-GFP hiPSC line), or C-KIT⁺ (PE) cells (after C-KIT staining) were then sorted out by FACS. Upon FACS sorting of the mCherry⁺/GFP⁺/PE⁺ cells, these cells were collected in a 1.5 mL Eppendorf tube with 500 μL 10% FBS. The tube was centrifuged at 300×g for 5 minutes and the supernatant was carefully removed. Cell pellet was then resuspended in an appropriate amount of hUBCM (with the addition of Y27632 at 10 μM final concentration) and cell density was measured by automatic cell counter. ˜2,000-20,000 cells were transferred into each well of a U-bottom 96-well low-attachment plate and an appropriate amount of hUBCM (with 10 μM Y27632 for the first 24 h) was added to the well to make the final volume of 100 μL per well. After 24 h, UB cell aggregate was formed and embedded into an 8 μL cold Matrigel droplet in another well of the U-bottom 96-well low-attachment plate and cultured in a 37° C. incubator following the same embedding procedure described above (with hUBCM). After approximately 7-10 days of culturing, epithelial tip structures could be seen budding out from the aggregate. WNT11-GFP expression was induced within 10 days in the PAX2-mCherry/WNT11-GFP hESC line derived organoid. These tip structures were dissected out and re-embedded into Matrigel to continue culture. From then, the iUB organoid was established and can be passaged stably following the procedures below.

### Human UB Organoid Expansion and Passaging

During the culture, human UBCM was renewed with fresh medium every two days. Both human UB organoid from primary RET⁺ UPC and iUB organoid from hPSCs were passaged every 6-10 days depending on the size. The passaging methods (manual or as single cells) are the same as defined above in the mouse UB organoid section, with the change of using hUBCM instead of mUBCM.

### CD Differentiation

Mouse CD Differentiation:

Mouse UB organoid was passaged at day 5 of expansion as single cells and 2,000 cells were seeded for continuing expansion. At day 10 of mUB expansion, mUBCM was removed and 150 μL 1×PBS was added and removed to wash the organoid. 150 μL of mouse CD differentiation medium (mCDDM) was then added to initiate mouse CD (mCD) differentiation (mCD differentiation Day 0). The organoid was cultured in a 37° C. incubator and medium was changed every two days or daily as needed for a total of seven days. No passage of the organoid was needed. At mCD differentiation Day 7, the mCD organoid was harvested for additional experiments.

Human CD Differentiation:

After human UB organoid expansion was stabilized (at least 25 days post FACS when UB organoid were growing stably) and reached an appropriate size (at least 900 μm diameter), hUBCM was removed and 150 μL 1×PBS was added and removed to wash the organoid. 150 μL of human CD differentiation medium (hCDDM) was added to start hCD differentiation (hCD differentiation Day 0). The organoid was cultured in 37° C. incubator and medium was changed every two days or daily if needed for a total of 14 days. At hCD differentiation Day 14, hCD organoid was harvested for analyses.

### Mouse Engineered Kidney Generation

The day before generating the mouse engineered kidney, 50-60 k 3D cultured mNPCs was seeded per 96-well to aggregate overnight. A small piece (with 6-10 branching tips) of Day 7-10 cultured mUB organoid was manually dissected out using sterile needles (similar to passaging UB organoid as small tips mentioned above) and inserted into a micro-dissected hole on a 3D cultured mNPC aggregate (first, a fine dissecting tweezer was used to hold/stabilized the mNPC aggregate sphere from one side, and a sterile needle was used to pierce a hole in the center of mNPC aggregate sphere from the other side; the small piece of mUB organoid was then carefully pushed into the hole using the needle; the NPC aggregate would then slowly wrap around the inserted mUB organoid autonomously overnight; all these procedures were done in a drop (80-100 μL) of kidney reconstruction medium (APEL2+0.1 μM TTNPB) with 10 μM Y27632 on an inverted 100 mm plastic petri dish cap, to ensure minimal movement of the aggregate/organoid during the procedures) in kidney reconstruction medium with 10 μM Y27632 to generate a engineered kidney precursor. This engineered kidney precursor was then carefully transferred into a well of a U-bottom 96-well low-attachment plate with 100 μL kidney reconstruction medium with 10 μM Y27632, using a P200 pipette with the top 0.5-1 cm of the tip cut, and cultured in 37° C. incubator (day 0). After 24 h (day 1), dead cells surrounding the precursor were removed by gently pipetting several times in the well using a P200 pipette with wide tip. The engineered kidney precursor was then transferred onto a 6-well transwell insert membrane using a wide-tip P200/P1000 pipette (depends on the size). Then 0.8-1 mL kidney reconstruction medium was added in the lower chamber of the transwell. The medium was changed every two days for a total of 7-10 days while the engineered kidney precursor maturation progressed. Then the engineered kidney was processed for further analyses.

### FACS

Cells were dissociated/prepared as described above. FACS sorting was performed on a BD FACS ARIA lllu cell sorter. Sorted cells were collected in a 1.5 mL Eppendorf tube with 500 μL 10% FBS on ice.

### RNA Isolation, Reverse Transcription and Quantitative PCR

Samples were dissolved in 100 μL TRIzol (Invitrogen, Cat. No. 15596018) and kept in −80° C. freezer. RNA isolation was performed using the Direct-zol RNA MicroPrep Kit (Zymo Research, Cat. No. R2062) according to the manufacturer's instructions. Reverse transcription was performed using the iScript Reverse Transcription Supermix (Bio-Rad, Cat. No. 1708841) following the manufacturer's instructions. qRT-PCR was performed using the Applied Biosystems PowerUp SYBR Green Master Mix (Thermo Fisher, Cat. No. A25777) and carried out on an Applied Biosystems Vii 7 RT-PCR system (Life Technologies). Validated gene-specific primers can be found in Table 14. Fold change was calculated from ΔCt using Gapdh as housekeeping gene.

TABLE 14
qRT-PCT Primer sequences.

| | | |
|---|---|---|
| Gene | | |
| Name | Forward Primer | Reverse Primer |

qRT-PCR Primers (Mouse)

| | | |
|---|---|---|
| Gapdh | CATGGCCTTCC | CCTGCTTCACCACCTT |
| | GTGTTCCTA | CTTGAT |
| | (SEQ ID NO: 31) | (SEQ ID NO: 32) |
| Wnt11 | TGTGCGGACAACCTC | ATGGCATTTACACTTC |
| | TAGC-AC | GTTTCCAG |
| | (SEQ ID NO: 33) | (SEQ ID NO: 34) |
| Ret | TGAGCCCTCGGC | GCCTCTGATGACAGC |
| | AACATTC | AATACTGGA |
| | (SEQ ID NO: 35) | (SEQ ID NO: 36) |
| Aqp2 | GCCACCTCCTTGG | TGTAGAGGAGGGA |
| | GATCTAT | ACCGATG |
| | (SEQ ID NO: 37) | (SEQ ID NO: 38) |
| Aqp3 | GGCGCTGGGATT | GCCAGAGACGAAA |
| | GTTTTTGG | AGCTCATT |
| | (SEQ ID NO: 39) | (SEQ ID NO: 40) |
| Foxi1 | AGTAC- | AGTCCAGTAATTCCC |
| | GTGGCCGACAACTTTC | TTTGCCT |
| | (SEQ ID NO: 41) | (SEQ ID NO: 42) |
| Tfcp2l1 | GCTGGAGAATCGGAA- | AAAACGACACGG |
| | GCTAGG | ATGATGCTC |
| | (SEQ ID NO: 43) | (SEQ ID NO: 44) |
| Atp6v1b1 | GGCTGTGACCCGAAAC- | CTCAGCATACTGG |
| | TACAT | GCAAACTT |
| | (SEQ ID NO: 45) | (SEQ ID NO: 46) |
| Slc4a1 | CTAG-TGGGC | TGCGGAACACTC |
| | CGGGCTAATTTT | TTTCTGTCA |
| | (SEQ ID NO: 47) | (SEQ ID NO: 48) |
| Slc26a4 | CCTTTGGTGTGGTAAA- | GACCGAACTGAA |
| | GACTCTC | CAGGTACTG |
| | (SEQ ID NO: 49) | (SEQ ID NO: 50) |

qRT-PCR Primers (Human)

| | | |
|---|---|---|
| GAPDH | GTGGACCTGACC | GGAGGAGTGGGT |
| | TGCCGTCT | GTCGCTGT |
| | (SEQ ID NO: 51) | (SEQ ID NO: 52) |
| PAX2 | CCCAAAGTGGTG | GAAAGGCTGCTGA |
| | GACAAGAT | ACTTTGG |
| | (SEQ ID NO: 53) | (SEQ ID NO: 54) |
| GATA3 | CGTCCTGTGCG | GTCCCCATTGGCA |
| | AACTGTCA | TTCCTCC |
| | (SEQ ID NO: 55) | (SEQ ID NO: 56) |
| LHX1 | CTTCTTCCGGTGT | TCATGCAGGTGAA |
| | TTCGGTA | GCAGTTC |
| | (SEQ ID NO: 57) | (SEQ ID NO: 58) |
| RET | TATCCTGGGATTC | TCTCCAGGTCTTT |
| | CTCCTGA | GCTGATG |
| | (SEQ ID NO: 59) | (SEQ ID NO: 60) |
| WNT11 | ATGTGCGGACAACC | GATGGAGCAGGA |
| | TCAGC-TAC | GCCAGACA |
| | (SEQ ID NO: 61) | (SEQ ID NO: 62) |
| CDH1 | ACTCGTAACGACGTT- | GGTCAGTATCAGC |
| | GCACCA | CGCTTTCAG |
| | (SEQ ID NO: 63) | (SEQ ID NO: 64) |
| EMX2 | AATGCGGCGAA | TTTAGACGAGGGT |
| | GACTCTGG | CGCTTGTTG |
| | (SEQ ID NO: 65) | (SEQ ID NO: 66) |
| HNF1B | GCTGTGACTCAGCTG- | TGTACTGATGCTGC |
| | CAGAACTC | TGGTATCTGTG |
| | (SEQ ID NO: 67) | (SEQ ID NO: 68) |
| ETV5 | CAGTCAACTTCAA- | TGCTCATGGCTA |
| | GAGGCTTGG | CAAGACGAC |
| | (SEQ ID NO: 69) | (SEQ ID NO: 70) |
| SOX9 | AGCGAACGCACAT | CTGTAGGCGATCTG |
| | CAAGAC | TTGGGG |
| | (SEQ ID NO: 71) | (SEQ ID NO: 72) |
| GFRA1 | AAGCACAGCTACGG | GTTGGGCTTCTCC |
| | AATGCT | CTCTCTT |
| | (SEQ ID NO: 73) | (SEQ ID NO: 74) |
| AQP2 | CTGG- | ATGTCTGCTGGCGTGAT |
| | TACAGGCTCTGGG | CTCATGGAG |
| | CCACATAA | (SEQ ID NO: 76) |
| | (SEQ ID NO: 75) | |
| AQP3 | AGACAGCCCCTTC | TCCCTTGCCCTGAA |
| | AGGATTT | TATCTG |
| | (SEQ ID NO: 77) | (SEQ ID NO: 78) |
| AQP4 | CATGGAAATCTTAC- | TCAGTCCGTTTGGA |
| | CGCTGGT | ATCACAG |
| | (SEQ ID NO: 79) | (SEQ ID NO: 80) |
| FOXI1 | AACTCACTGAC- | CCATAGCTGAGC |
| | CTTCAACTCCT | ATGTTGGT |
| | (SEQ ID NO: 81) | (SEQ ID NO: 82) |

### Immunofluorescence

Whole-Mount Staining:

Samples were fixed in 4% PFA for 45 minutes at 4° C. in Eppendorf tubes with gentle shaking (UB/CD organoid, 200 μL PFA) or 10 minutes at room temperature on transwell insert membrane (kidney reconstruct, 1 mL total PFA on and below the membrane). They were then washed four times in 0.8-1 mL 1×PBS (Corning, Cat. No. 21-040-CV) for total 30 minutes at 4° C. or room temperature (after the washes, kidney reconstructs on transwell membrane were cut out and transferred into Eppendorf tubes). After the washes, samples were blocked in blocking solution (0.100 PBST containing 300 BSA) for 1-2 hours at 4° C. with gentle sharking, followed by primary antibody staining (primary antibodies were diluted in blocking solution) at 4° C. overnight. On the second day, samples were washed three times with 800 μL 0.1% PBST for total 3 hours at 4° C. with gentle sharking. Secondary antibodies diluted in blocking solution were added and samples were incubated at 4° C. overnight. On the third day, samples were washed three times with 800 μL PBST for total 3 hours at 4° C. with gentle sharking. Lastly, samples were mounted in mounting medium onto glass slides for imaging.

Cryo-Section Staining:

Samples were fixed and washed as described above. They were then transferred into a plastic mold and embedded in OCT Compound (Scigen, Cat. No. 4586K1) and froze in −80° C. for 24 hours to make a cryo-block. The cryo-blocks were sectioned using Leica CM1800 Cryostat. The sectioned slides were then blocked for 30 minutes at room temperature followed by one hour of primary antibodies staining at room temperature. The slides were then washed four times with PBST for five minutes, and then secondary staining for 30 minutes. After the secondary staining, the slides were washed four times with PBST for five minutes and mounted with mounting medium.

WNT11-GFP/PAX2-mCherry Dual Reporter hESC Line Generation

CRISPR-Cas9 based genome editing was used to insert 2A-EGFP-FRT-PGK-Neo-FRT or 2A-mCherry-loxP-PGK-Neo-loxP cassette downstream of the stop codon (removed) of endogenous WNT11 or PAX2 gene, respectively. DNA sequences ˜1 Kb upstream and ˜1 Kb downstream of endogenous WNT11 (upstream F: CCGGAATTCGAC-GTAATCATTCCACTGACC (SEQ ID NO:1); upstream R: TACGAGCTCCTTGCAGA-CATAGCGCTCCAC (SEQ ID NO:2); downstream F: CGCGTCGACGGCCCTGCCCTAC-GCCCCA (SEQ ID NO:3); downstream R: CCCAAGCTTTGCCTGGAAACTGGA-GAGCTCCCTC (SEQ ID NO:4)) and PAX2 (upstream F: GAAGTCGACTTTCCACCCATT-AGGGGCCA (SEQ ID NO:5); upstream R: TATGCTAGCGTGGCGGTCATAGGCAGCGG (SEQ ID NO:6); downstream F: TATACGCGTTTACCGCGGGGACCACATCA (SEQ ID NO:7); downstream R: GACGGTACCAGTAACTGCTGGAGGAAGAC (SEQ ID NO:8)) stop codon were cloned upstream and downstream of 2A-EGFP-FRT-PGK-Neo-FRT or 2A-mCherry-loxP-PGK-Neo-loxP cassette respectively to facilitate homologous recombination. 2A-EGFP fragment was cloned from pCAS9_GFP (Addgene #44719) and the FRT-PGK-Neo-FRT cassette was cloned from pZero-FRT-Neo3R (kindly provided by Dr. Keiichiro Suzuki). 2A-mCherry-loxP-Neo-loxP fragment was cloned from Nanog-2A-mCherry plasmid (Addgene #59995). The different fragments were then cloned to pUC19 plasmid to make the complete donor plasmids for both knockin experiments. gRNA oligos for WNT11 (F: CAC-CGGTCCTCGCTCCTGCGTGGGG (SEQ ID NO:9); R: AAACCCCCACGCAG-GAGCGAGGACC (SEQ ID NO:10)) and PAX2 (F: CACCGATGACCGCCACTAG-TTACCG (SEQ ID NO:11); R: AAACCGGTAACTAGTGGCGGTCATC (SEQ ID NO:12)) were synthesized and cloned into the lentiCRISPR v2 plasmid (Addgene #52961). First, both donor and gRNA plasmids for PAX2 reporter KI were transfected into the H1 hESCs using the Lipofectamine 3000 Transfection Reagent (Invitrogen, Cat. No. L3000015). Neomycin-resistant single cell colonies were picked up manually and genotyping was performed based on PCR. PCR primers see Table 15. Clones with biallelic knockin of PAX2-mCherry were chosen for second round screen where plasmid encoding Cre was delivered to allow the transient expression Cre, whose activities excise the loxP-flanked PGK-Neo cassette from the knockin alleles. PCR was performed to identify single cell clones in which PGK-Neo cassettes were excised from both alleles. Then the same strategy was used to knock in WNT11 reporter based on the successful biallelic PAX2-mCher knockin clones.

TABLE 15
Genotyping primer sequences for detecting
PAX2-mCherry and WNT11-GFP
knockin (KI).
PCR Primers

| Reporter Name | Forward Primer | Reverse Primer |
|---|---|---|
| PAX2-mCherry | TCCCATTTCCA | GCATCAAGTA |
| KI | CCCATT-AGGG | ACTGCTGGAG |
| | GCCA | -GAAGACC |
| | (SEQ ID NO: 13) | (SEQ ID NO: 14) |
| WNT11-GFP KI | ACAAGAC | TGAGGGTCCT |
| | ATCCAAC- | TGAGCAGAGT |
| | GGAAGC | (SEQ ID NO: 16) |
| | (SEQ ID NO: 15) | |

### RNA Sequencing

Adult mouse CD cells were FACS isolated (Hoxb7-Venus⁺) from adult (˜2 month old) Hoxb7-Venus mouse kidneys. All samples were collected and lysed in TRIzol reagent and stored under −80° C. Total RNA was extracted using Direct-zol RNA MicroPrep Kit (Zymo). cDNA library was prepared using KAPA Stranded mRNA-Seq Kit (KAPA Biosystems). RNA-Sequencing was performed by the Children's Hospital Los Angeles Sequencing Core.

### Gene Editing in Mouse UB Organoid

Gene Over-Expression:

Lentiviral infection was used to overexpress GFP in E11.5 mUB cells. Lentivirus was first concentrated 100× using Lenti-X Concentrator kit from Takara (Cat #631231). Concentrated lentivirus was aliquoted and stored in −80° C. before use. The lentivirus was used at 1× final concentration together with 10 μM Polybrene (Sigma-Aldrich, Cat. No. TR-1003-G) diluted in mUBCM (with 10 μM Y27632). 100 μL virus-UBCM mixture was added to the U-bottom 96-well low-attachment plate well with single cells suspension prepared from 8-10 E11.5 mUBs. The UBs and virus were centrifuged together at 800 g for 30 minutes for spinfection at room temperature. After the spinfection, the virus-UBCM mixture was removed and the infected UB cells were washed three times with PBS, then aggregated overnight and embedded in Matrigel and cultured in mUBCM in 37° C. incubator following standard UB organoid culture procedures described above. 200 μg/mL G-418 (Invitrogen, Cat. #10131027) was added to the culture to select for UB cells that have been successfully infected. The resulting UB aggregate self-organized into typical branching organoid 4-5 days after infection.

Gfp Knockout:

An E11.5 mUB single cell suspension from the Rosa26-Cas9/GFP background was used and lentiviral vectors were constructed using the lentiGuide-puro vector system (Addgene #52963) following standard protocol to make lentiviruses expressing three different gRNAs targeting GFP (gRNA sequences: F1: CACCGAAGGGCGAGGAGCTGTTCAC (SEQ ID NO:17), R1: AAACGTGAACAGCTCCTCGCCCTTC (SEQ ID NO:18); F2: CAC-CGCTGAAGTTCATCTGCACCAC (SEQ ID NO:19), R2 AAACGTGGTG-CAGATGAACTTCAC (SEQ ID NO:20); F3: CACCGGGAGCGCACCATCTTCTTCA (SEQ ID NO:21), R3: AAACTGAAGAAGATGGTGCGCTCCC (SEQ ID NO:22)) with the Cas9 cutting site 100-150 bp apart, or three non-targeting gRNAs as control. The 100× concentrated lentivirus were used at 5× together with 10 μM Polybrene diluted in mUBCM (with 10 μM Y27632). 100 μL virus-UBCM mixture was added to the U-bottom 96-well low-attachment plate well to combine them with 10 E11.5 mUBs that have been dissociated into single cells. The UB cells and virus were centrifuged at 800×g for 30 minutes for spin-infection. After the spin, virus-UBCM mixture was removed and fresh virus-UBCM mixture was added into the same well and the UB cells were spin-infected for another 30 minutes at 800 g. Then, virus-UBCM mixture was removed and the infected UBs were washed three times with PBS, then aggregated overnight and embedded in Matrigel and cultured in mUBCM in 37° C. incubator following standard UB organoid culture procedures described above. 0.2 μg/mL puromycin was added to the medium to select for UB cells that have been successfully infected. The UB aggregate self-organized into typical branching organoid by 4-5 days post-infection.

Ret/RET Knockout in Mouse/Human UB Organoid:

Day 5 cultured mUB organoids (wildtype or any background) or stably expanded hUB organoids were dissociated into single cells following the method described above. gRNA oligos targeting mouse or human Ret/RET were synthesized and cloned into the lentiCRISPR v2 plasmid (Addgene #52961) (mRet gRNA: F1: CACCGGAAGCTCGGCAC-TTCTCCAG (SEQ ID NO:23); R1: AAACCTGGAGAAGTGCCGAGCTTCC (SEQ ID NO:24); F2: CACCGCTGTATGTAGACCAGCCAGC (SEQ ID NO:25); R2: AAAC-GCTGGCTGGTCTACATACAGC (SEQ ID NO:26). hRET gRNA: F1: CACCGG-TAGAGGCCCAATGCCACTG (SEQ ID NO:27); R1: AAACCAGTGGCATTGGGCCTC-TACC (SEQ ID NO:28); F2: CACCGAAGCATCCCTCGAGAAGTAG (SEQ ID NO:29); R2: AAACCTACTTCTCGAGGGATGCTTC (SEQ ID NO:30)). The same gRNA oligos cloned into the lentiGuide-puro vector system (Addgene #52963) that don't express the Cas9 enzyme were used as negative control. Lentiviruses with these vectors were generated following standard protocol. The 100× concentrated lentivirus were used at 2× together with 10 μM Polybrene diluted in m/hUBCM (with 10 μM Y27632). 100 μL virus-UBCM mixture was added to the U-bottom 96-well low-attachment plate well to combine them with 15,000-20,000 m/hUB single cells. The UB cells and virus were centrifuged at 800×g for 15 minutes for spin-infection. Then, virus-UBCM mixture was removed and the infected UBs were washed three times with PBS, then aggregated overnight and embedded in Matrigel and cultured in m/hUBCM in 37° C. incubator following standard UB organoid culture procedures described above. Puromycin (0.2 g/mL for mouse and 0.3 μg/mL for human) was added to the medium two-days post-infection to select for UB cells that have been successfully infected. The UB aggregate self-organized into typical branching organoid by 2-6 days post-infection. Mouse organoids were harvested 6 days post-infection and human organoids were harvested 10-12 days post-infection for further analysis.

Human UB Organoid Cryopreservation

Human UB orgaonid were cultured until they reached the size ready for passaging. It was transferred onto 100 mm Petri dish lid and Matrigel was removed following the method described above. Organoid was then cut into 4-6 pieces using sterile needles and transferred into an Eppendorf tube. Extra medium in the tube was removed and replaced with 200 L hUBCM with 10 μM Y27632 supplemented with DMSO at 10%. The medium and organoid pieces were then split into two cryogenic tubes for cryopreservation. To revive the organoid, the frozen cryogenic tube was thawed in 37° C. water bath. Medium in the tube was removed and replaced with 50-100 μL fresh hUBCM with 10 μM Y27632. Each organoid piece was then embedded into 8 μL Matrigel and cultured in hUBCM (with 10 μM Y27632 for the first 24 h) following the method described above.

### Quantification and Statistical Analysis

### RNA-Seq Data Analysis

RNA sequencing data was analyzed using Partek Flow, including published dataset of interstitial progenitor cells and nephron progenitor cells (Lindstrom et al., 2018), ureteric tip and trunk cells (Rutledge et al., 2017). FASTQ files were trimmed from both ends based on a minimum read length of 25 bps and a shred quality score of 20 or higher. Reads were aligned to GENCODE mm10 (release M24) using STAR 2.5.3a. Aligned reads were quantified to the Partek E/M annotation model. Gene counts were normalized by adding 1 then by TMM values. Samples were filtered to include differentially expressed genes of UB tip compared to UB trunk, with false discovery rate<=0.01, fold change<−4 or >4, total counts>=10, and p-value<0.05, resulting in 1413 UB tip/trunk signature genes. Then, hierarchical clustering was produced on by clustering samples and features with average linkage cluster distance and Euclidean point distance. Principle component analysis (PCA) was performed using the EDASeq R/Bioconductor packages and the plot was rendered with the ggplot2 R package.

### Image Quantification for UB/CD Marker Gene Expression in the UB/CD Organoids

Whole-mount immunostaining images for mouse UB organoids, mouse CD organoids, or human UB organoids were used for the quantification of various marker gene expression. ImageJ software was used to count positive cells. 3 different fields of view per organoid were randomly selected to count the number of positively stained cell numbers (positive for marker genes) and total cell numbers (DAPI+). Percentage was calculated by the number of cells that are positive for different UB/CD marker genes divided by the total DAPI+ cell numbers. At least 500 cells in total were counted. Error bars represent standard derivation between different field views.

### Data and Code Availability

RNA-seq data have been submitted to Gene Expression Omnibus (GEO) with accession number GSE149109.

TABLE 16
Medium recipe of stepwise directed differentiation to ND cells
(D 1 to D 7 from hPSC), listing supplements to a basal medium,
DMEM/F12 (1:1) (1X), Invitrogen, Cat. No. 11330-032.

| | | Final | |
|---|---|---|---|
| | | Concen- | |
| Stages | Reagent Name | tration | |
| Common supplements | GlutaMAX-I (100×) | 1× | |
| for all three stages | MEM NEAA (100×) | 1× | |
| | 2-Mercaptoethanol (55 mM) | 0.1 | mM |
| | Pen Strep (100×) | 1× | |
| | B-27 Supplement (50×), | 1× | |
| | minus vitamin A | | |
| | ITS Liquid Media | 1× | |
| | Supplement (100×) | | |
| ME Stage (D 1-D 3) | Activin A | 50 | ng/ml |
| (version 1, FIG. 4) | CHIR99021 | 3 | μM |
| ME Stage (D 1-D 3) | LDN-193189 | 10 | nM |
| (version 2, FIG. 5) | CHIR99021 | 4.5 | μM |
| UB-I Stage (D 3-D 5) | FGF2 | 200 | ng/ml |
| | TTNPB | 0.1 | μM |
| | LDN-193189 | 30 | nM |
| | A83-01 | 0.2 | μM |
| UB-II Stage (D 5-D 7) | FGF2 | 200 | ng/ml |
| | TTNPB | 0.1 | μM |
| | LDN-193189 | 30 | nM |

Various embodiments of the invention are described above. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising” is used herein to describe and claim the invention, the present invention or embodiments thereof may alternatively be described using alternative terms such as “consisting of” or “consisting essentially of.”

1. A method of generating a renal ureteric bud (UB) organoid, comprising:
culturing a population of UB progenitor cells (UPCs) in a UB culture medium to induce a branching morphology of the UPCs, thereby forming an UB organoid,
wherein the UB culture medium comprises a basal medium and supplements, said supplements comprise all, or one or more, of: LDN-193189, TTNPB, CHIR99021, Janus-associated kinase inhibitor I (JAK inhibitor I), glial cell-derived neurotrophic factor (GDNF), A83-01, R-spondin 1, a fibroblast growth factor (FGF), and SB202190.

2. The method of claim 1, wherein the UPCs are obtained from human pluripotent stem cells (PSCs) or human fetal kidney and express one or both of WNT11 and RET, and wherein the supplements of the UB culture medium comprise LDN-193189, TTNPB, CHIR99021, JAK inhibitor I, GDNF, A83-01, R-spondin 1, fibroblast growth factor (FGF) 7, SB202190, and epidermal growth factor (EGF).

3. The method of claim 2, wherein the supplements of the UB culture medium do not comprise Y27632.

4. The method of claim 1, wherein the UPCs are obtained from mouse fetal kidney or from pluripotent stem cells (PSCs), and the UPCs are positive in one or both of markers of Ret and Wnt11; and wherein the supplements of the UB culture medium comprise FGF9, TTNPB, CHIR99021, GDNF, LDN-193189, A83-01, JAK Inhibitor I, SB202190, and R-Spondin 1, said supplements do not comprise EGF.

5. The method of claim 1, wherein the basal medium of the UB culture medium comprises DMEM/F12 (1:1) and is further supplemented with L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution, and wherein the culturing of the UPCs comprises culturing in a Matrigel.

6. The method of claim 1, further comprising obtaining the population of UPCs by:
cultivating human PSCs in the presence of:
mTeSR™1 medium (TeSR) and CloneR (CR) for a first period of time,
ME medium for a second period of time,
UB-I medium for a third period of time,
UB-II medium for a fourth period of time; and
sorting KIT+ cells from the cultivated human PSCs, thereby obtaining the UPCs,
wherein the ME medium comprises supplements of LDN-193189 and CHIR99021, the UB-I medium comprises supplements of FGF2, TTNPB, LDN-193189, and A83-01, and the UB-II medium comprises supplements of FGF2, TTNPB, and LDN-193189.

7. The method of claim 6, wherein the cultivation is in a sequential order:
the first period of time of about 1 day in the presence of the TeSR and the CR,
then the second period of time of about 2 days in the presence of the ME medium and absence of the TeSR and the CR,
then the third period of time of about 2 days in the presence of the UB-I medium and absence of the ME medium, the TeSR, and the CR, and
followed by the fourth period of time of about 2 days in the presence of the UB-II medium and absence of the UB-I medium, the ME medium, the TeSR, and the CR.

8. The method of claim 1, wherein at least 99%, 98%, 97%, 96%, or 95% of cells in the branching morphology are positive in one or more markers for UPCs, one or more UPC regulators, and/or one or more UB lineage markers,
wherein the markers for UPCs comprise Ret and Wnt11, the UPC regulators comprise Ret, Etv5, and Sox9, and the UB lineage markers comprise Gata3, Pax2, Krt8, and Cdh1.

9. The method of claim 1, further comprising resecting a tip portion of cells from the branching morphology, and culturing the tip portion of cells in a fresh volume of the UB culture medium to induce branching morphology, thereby generating a subsequent passage of the UB organoid.

10. The method of claim 9, which is repeated for up to 3 weeks when the population of UPCs are obtained from mouse fetal kidney, or wherein the method is repeated for at least 100 days when the population of UPCs are obtained from human fetal kidney, or wherein the method is repeated for at least 70 days when the population of UPCs are obtained from human PSCs.

11. A method of generating a renal collecting duct (CD) organoid, comprising:
culturing a renal ureteric bud (UB) organoid in a CD differentiation medium, said CD differentiation medium comprises a basal medium and supplements, thereby generating a CD organoid,
wherein the renal UB organoid comprises, or is generated with, human ureteric bud progenitor cells (UPCs), and the supplements of the CD differentiation medium comprise aldosterone, vasopressin, and KNOCKOUT serum replacement (KSR), or
wherein the renal UB organoid comprises, or is generated with, mouse ureteric bud progenitor cells (UPCs), and the supplements of the CD differentiation medium comprises FGF9, Y27632, DAPT, PD0325901, aldosterone, and vasopressin.

12. A method of generating a renal collecting duct (CD) organoid, comprising:
generating a renal ureteric bud (UB) organoid according to a method of claim 1, and
culturing a renal ureteric bud (UB) organoid in a CD differentiation medium, said CD differentiation medium comprises a basal medium and supplements, thereby generating a CD organoid,
wherein the renal UB organoid comprises, or is generated with, human ureteric bud progenitor cells (UPCs), and the supplements of the CD differentiation medium comprise aldosterone, vasopressin, and KNOCKOUT serum replacement (KSR), or
wherein the renal UB organoid comprises, or is generated with, mouse ureteric bud progenitor cells (UPCs), and the supplements of the CD differentiation medium comprises FGF9, Y27632, DAPT, PD0325901, aldosterone, and vasopressin.

13. The method of claim 11, wherein the culture in the CD differentiation medium comprises culturing for 7 days or longer to form an elongated CD organoid morphology, and/or the CD organoid is characterized by elevated expressions of a principal cell (PC)-specific marker and/or an intercalated cell (IC)-specific marker.

14. The method of claim 11, wherein the basal medium of the CD differentiation medium comprises DMEM/F12 (1:1) and is further supplemented with L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution.

15. The method of claim 13, wherein the PC-specific marker comprises one or more of AQP2 and AQP3, and the IC-specific marker comprises one or more of FOXI1, ATP6V1B1, SLC4A1/AE1, and SLC26A4/PENDRIN.

16. A method of generating ureteric bud progenitor cells (UPCs) from human pluripotent stem cells (PSCs), comprising:
cultivating the human PSCs in the presence of:
mTeSR™1 medium (TeSR) and CloneR (CR) for a first period of time,
ME medium for a second period of time,
UB-I medium for a third period of time,
UB-II medium for a fourth period of time; and
sorting KIT+ cells from the cultivated human PSCs, thereby obtaining the UPCs,
wherein the ME medium comprises supplements of LDN-193189 and CHIR99021, the UB-I medium comprises supplements of FGF2, TTNPB, LDN-193189, and A83-01, and the UB-II medium comprises supplements of FGF2, TTNPB, and LDN-193189.

17. A method of generating an engineered kidney, comprising:
obtaining a tip portion of cells from a branch of a renal ureteric bud (UB) organoid,
combining the tip portion of the renal UB organoid with nephron progenitor cells (NPCs) in one culture, and
cultivating the combination in a kidney reconstruction medium, to generate a tubular network with connected nephron-like cell types and a collecting duct,
wherein the kidney reconstruction medium comprises all, or one or more, of: TTNPB, APEL2, and Y27632.

18. The method of claim 17, wherein the combining step comprising inserting the tip portion of the renal UB organoid into an excavated cavity of a culture of the NPCs, and the cultivating step comprising culturing in an air-liquid interface.

19. The method of claim 17, wherein the method does not comprise interxtitial progenitor cells in the generation of the engineered kidney.

20. An ureteric bud (UB) organoid, generated by a method of claim 1, comprising at least 99%, 98%, 97%, 96%, or 95% of UB progenitor cells, which express one or more markers for UPCs, one or more UPC regulators, and/or one or more UB lineage markers, wherein the markers for UPCs comprise Ret and Wnt11, the UPC regulators comprise Ret, Etv5, and Sox9, and the UB lineage markers comprise Gata3, Pax2, Krt8, and Cdh1.

21. A population of ureteric bud (UB) organoids for ex vivo modeling of a kidney disease, comprising: a plurality of the UB organoids of claim 20, wherein at least a population of cells in the UB organoids comprise at least one edited gene, wherein the edited gene comprises a mutation, an overexpression, a down regulation, a knock out, or a combination thereof.

22. A method of screening for a candidate drug for treating, reducing the incidence or severity of a kidney disease and/or for promoting kidney regeneration, comprising:
contacting a molecule of interest with an UB organoid generated by a method of claim 1; and
measuring a level of a biomarker transcribed or expressed in the UB organoid before with contact of the molecule of interest, and
measuring a level of the biomarker transcribed or expressed in the UB organoid in the presence of the molecule of interest.

23. A method of screening for a candidate drug for treating, reducing the incidence or severity of a kidney disease and/or for promoting kidney regeneration, comprising:
contacting a molecule of interest with an engineered kidney generated by the method of claim 17; and
measuring a level of a biomarker transcribed or expressed in the engineered kidney before contact of the molecule of interest, and
measuring a level of the biomarker transcribed or expressed in the engineered kidney in the presence of the molecule of interest.

24. A set of supplements comprising:
(a) LDN-193189, TTNPB, CHIR99021, Janus-associated kinase inhibitor I (JAK inhibitor I), glial cell-derived neurotrophic factor (GDNF), A83-01, R-spondin 1, fibroblast growth factor (FGF) 7, SB202190, and epidermal growth factor (EGF) for cultivating human ureteric bud (UB) progenitor cells in a medium to generate UB organoids,
or
(b) FGF9, TTNPB, CHIR99021, GDNF, LDN-193189, A83-01, JAK Inhibitor I, SB202190, and R-Spondin 1, said supplements do not comprise EGF, for cultivating mouse ureteric bud (UB) progenitor cells in a medium to generate UB organoids.

25. The set of supplements of claim 24, for cultivating human ureteric bud (UB) progenitor cells, which does not comprises Y27632.

26. A medium composition comprising:
(a) a basal medium, L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution, and the set of supplements of claim 24 for cultivating human ureteric bud (UB) progenitor cells to generate UB organoids,
or
(b) a basal medium, L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution, and the set of supplements of claim 24 for cultivating mouse ureteric bud (UB) progenitor cells to generate UB organoids.

27. (canceled)

28. (canceled)

29. A medium composition comprising:
(a) a basal medium and supplements of aldosterone, vasopressin, and KNOCKOUT serum replacement (KSR), and L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution, for differentiating human ureteric bud (UB) organoids, or human UB progenitor cells, into a renal collecting duct (CD) organoid,
or
(b) a basal medium and supplements of FGF9, Y27632, DAPT, PD0325901, aldosterone, and vasopressin, and L-alanyl-L-glutamine (GlutaMAX-I), MEM non-essential amino acids solution, 2-mercaptoethanol, penicillin streptocycin solution, B-27 devoid of vitamin A, and insulin-transferrin-sodium selenite (ITS) solution, for differentiating mouse ureteric bud (UB) organoids, or mouse UB progenitor cells, into a renal collecting duct (CD) organoid.

30. (canceled)
